(19) United States
(12) Patent Application Publication
(10) Pub. No.: US 2010/0210829 A1
(43) Pub. Date: Aug. 19, 2010

(54) Compositions and Methods for Treating Diseases, Disorders, or Conditions Characterized by Myelin Degeneration, Myelin Deficiency or Loss

(75) Inventors: Thomas Linnemann, San Francisco, CA (US); Lorianne Masuoka, Oakland, CA (US); Srinivas Kothakoto, Pacifica, CA (US); Stephen Doberstein, San Francisco, CA (US)

Correspondence Address: FIVE PRIME THERAPEUTICS, INC., 1650 Owens Street, Suite 200, San Francisco, CA, 94158, US

(73) Assignee: Five Prime Therapeutics, Inc., San Francisco, CA (US)

(21) Appl. No.: 12/530,447

(22) PCT Filed: Mar. 7, 2008
(86) PCT No.: PCT/US08/03090

§ 371 Date: Apr. 20, 2010

Related U.S. Application Data
(60) Provisional application No. 60/893,790, filed on Mar. 8, 2007.

(51) Int. Cl. C07H 21/04

(52) U.S. Cl. 536/23.5

(57) ABSTRACT

Provided herein are novel polypeptides, polynucleotides and compositions containing such, and to methods useful in treating diseases, disorders, or conditions of the central nervous system (CNS) associated with demyelination. The novel polypeptides, polynucleotides stimulate oligodendrocyte precursor cell (OPC), and are capable of promoting myelination or remyelination in a subject.

### TECHNICAL FIELD

This invention is in the field of neurology. Specifically, the invention relates to the discovery of new polypeptides, polynucleotides and compositions containing such, and to methods useful in treating diseases, disorders, or conditions of the central nervous system (CNS), which are associated with demyelination. These compositions and methods are capable of stimulating and/or promoting myelination or remyelination in a subject. The compositions and methods embodied in the present invention are particularly useful in treating neurological diseases, such as, multiple sclerosis.

### BACKGROUND OF THE INVENTION

Demyelination is a damaging condition that is characterized by a reduction of myelin in the nervous system. Myelin is a vital component of the central (CNS) and peripheral (PNS) nervous systems, which encase the axons of neurons and forms an insulating layer known as the myelin sheath. The presence of the myelin sheath enhances the speed and integrity of a nerve signal in the form of an electric potential propagating down the neural axon and provides trophic support for neurons. The loss of myelin sheath produces significant impairment in sensory, motor and other types of functioning as nerve signals reach their targets either too slowly, asynchronously (for example, when some axons in a nerve conduct faster than others), intermittently (for example, when conduction is impaired only at high frequencies), or not at all. In addition, the demyelinated neurons are prone to degeneration.

In demyelinating diseases so called shadow plaques have been identified, which represent areas of remyelination, indicating endogenous repair capabilities of the CNS. (Lassmann, Brain Pathol., 15:217-222 (2005); Pfeiffer, et al., Trends Cell Biol., 3:191-197 (1993)).

Demyelination is manifested in a large number of hereditary and acquired diseases of the CNS and PNS. These diseases include, for example, Multiple Sclerosis (MS). MS is the most common cause of chronic neuroligic disability with a major onset between 20 and 40 years of age, affecting approximately 2,500,000 people worldwide and approximately 400,000 people in the United States. The disease is characterized clinically by relapses and remissions, and leading eventually to chronic disability. The earlier phase of multiple sclerosis is characterized by the autoimmune inflammatory strike against myelin sheath leading to paralysis, lack of coordination, sensory disturbances and visual impairment. The subsequent chronic progressive phase, which is resistant to treatment is characterized by fewer immune attacks and an extensive lesion burden, which is believed to result in a loss of neurons. (Hauser and Oksenberg, Neuron, 52:61-76 (2006); Franklin, Nat. Rev. Neurosci., 3:705-714 (2002); Bruck et al., J. Neurol. Sci., 206:181-185 (2003)). The precise etiology and pathogenesis of MS remains largely unknown. However, pathologic, genetic, and immunologic features have been identified that suggest MS involves inflammatory and autoimmune basis. (See, for example, Hafler et al., Immunol. Rev., 100:307-332 (1987)).

Current treatments of MS are primarily directed against an immune component. While they are effective in reducing immune attacks against the myelin sheaths, as characterized by relapses, they eventually fail to control the disease and less than 20% of MS patients have no functional limitations 15 years after disease onset. Thus, there remains a considerable need for treatments which provide for the induction of remyelination in diseases characterized by demyelination.

### BRIEF DESCRIPTION OF THE FIGURES

FIG. 1 shows a microscopic view of oligodendrocyte precursor cells (OPC) in culture taken at 100× magnification (scale bar: 10 um (micrometers)).

FIG. 2 is a graphical representation of the two different oligodendrocyte precursor cells (OPC) screening assays. FIG. 2A shows OPC treated with varying amounts of recombinant human FGF-2 (rhFGF-2) (x-axis in log₁₀ ng/ml) and the relative ATP signal measured by luminescence (y-axis) after incubation of the cells for 4 days at 37° C. in a 5% CO₂ chamber. The EC₅₀ was 66 picomolar (pM) and the signal amplitude was 2.3. FIG. 2B shows OPC treated with varying amounts of recombinant human rhFGF-2 and the mean and standard deviation of the total number of nuclei in 5 different microscopic fields as counted by microscopy. The EC₅₀ was 130 pM and the signal amplitude was 1.8.

FIG. 3 is a graphical representation of ATP levels of OPC cultures after stimulation with conditioned supernatants. Shown is the relative ATP amount in OPC cell cultures in a 96 well plate. Recombinant external protein controls (rBMP-4, rFGF-2 and rIGF-1) and internal controls (IGF-1 and FGF-2) are marked. In addition a representative hit (CLN00528015) is shown.

### BRIEF DESCRIPTION OF THE TABLES

Table 1 provides a list of oligodendrocyte precursor cell (OPC) hits as identified through a proliferation assay and measured with a CelltiterGlo Kit as described in Example 5. Test proteins that yielded a positive value, averaged or otherwise, which were at least 2.0 sigma above the median, calculated in accordance to the formula shown in Example 5, were each designated as an “OPC hit.” Column 1 shows an internal clone identification number. Column 2 shows the cluster annotation of each listed protein. Column 3 shows the sigma value from the mean for each listed protein.

Table 2 shows all OPC hits of the present invention. Column 1 shows an internal clone identification number. Column 2 shows a cluster ID number of an identified hit, where each cluster represents a unique gene locus on the human chromosome. Although it would be clear for one skilled in the art to understand the convention in connection with the construct/protein ID, for convenience, we provide a brief explanation. For example, for Expression Clone CLN00874596, categorized in Cluster 195530, the Parent Protein ID is NP_{—}003719, which means that the full-length sequence of this protein may be found in the NCBI database under this accession number. Column 3 shows the public accession number of the polypeptides that represent the full length protein (Parent protein ID). Column 4 shows the public accession numbers of the protein fragments identified (Construct/Protein ID), each fragment being identified by the beginning and the ending amino acid residues, where amino acid residue 1 represents the first amino acid residue of the specified full-length protein. In certain instances, an ECOR1 linker was present linking two different protein fragments. Column 5 indicates the classification of a protein as either secreted, a single transmembrane protein type I or type II. Column 6 shows the name of the Pfam domain when present. Column 7 shows the name of the gene when present. Column 8 shows the annotation of each listed protein. Column 9 shows the sigma value from the mean for each listed protein.

Table 3 shows the SEQ ID NOS. for molecules of the invention (SEQ ID NOS: 1-226). Column 1 shows internally designated identification numbers (Patent ID) of the expression clones (each containing a cDNA encoding a designated polypeptide in a vector) of certain of the OPC hits identified, for example, in Tables 1 and 2. Column 2 shows the SEQ ID NOs. of the amino acid sequences (P1 SEQ ID) for the proteins shown in Column 4. Column 3 shows the SEQ ID NOS. of the nucleic acid sequences (N1 SEQ ID) that encode the corresponding polypeptide sequences shown in Column 2. Column 4 shows the public accession numbers of the protein fragments identified (Construct/Protein ID), each fragment being identified by the beginning and the ending amino acid residues, where amino acid residue 1 represents the first amino acid residue of the specified full-length protein. In certain instances, an ECOR1 linker was present linking two different protein fragments.

Table 4 shows Pfams of the proteins of the invention if present. Column 1 shows internally designated identification numbers (Patent ID) of the expression clones (each containing a cDNA encoding a designated polypeptide in a vector) of certain of the OPC hits identified, for example, in Tables 1 and 2. Column 2 shows an internal clone identification number. Column 3 shows the public accession numbers of the protein fragments identified (Construct/Protein ID), each fragment being identified by the beginning and the ending amino acid residues, where amino acid residue 1 represents the first amino acid residue of the specified full-length protein. In certain instances, an ECOR1 linker was present linking two different protein fragments. Column 4 shows the name of the Pfam domain when present, and Columns 5 and 6 shows the coordinates of the Pfam domains. Start and End Coordinates of the Pfam domains, in amino acids, are listed in terms of the positions of the beginning and ending amino acid residues, beginning with “1” for the first amino acid residue at the N-terminus of the full-length polypeptide. Pfam represents the protein family, an explanation of which can be found at http://pfam.wustl.edu. Column 7 shows the length of the protein identified in amino acids.

Table 5 shows the Tree vote and mature polypeptide positions of the proteins of the invention. Column 1 shows internally designated identification numbers (Patent ID) of the expression clones (each containing a cDNA encoding a designated polypeptide in a vector) of certain of the OPC hits identified, for example, in Tables 1 and 2. Column 2 shows an internal clone identification number. Column 3 shows the public accession numbers of the protein fragments identified (Construct/Protein ID), each fragment being identified by the beginning and the ending amino acid residues, where amino acid residue 1 represents the first amino acid residue of the specified full-length protein. In certain instances, an ECOR1 linker was present linking two different protein fragments. Column 4 shows the result of an algorithm that predicts whether a polypeptide of the invention is secreted (Tree Vote). This algorithm is constructed on the basis of a number of attributes including hydrophobicity, two-dimensional structure, prediction of signal sequence cleavage site, and other parameters. Based on such an algorithm, a sequence that has a secreted tree vote of approximately 0.5 is believed to be a secreted protein. Column 5 shows the coordinate positions of the amino acid residues comprising the signal peptide sequences (Signal Peptide Coords./Signal Sequence Position) of proteins that include signal peptide sequences. Column 6 shows the coordinate positions of the amino acid residues comprising the mature protein sequences (Mature Protein Coords.) of the cDNA clones of the invention following cleavage of the signal peptide.

Table 6 shows examples of demyelinating pathologies.

Appendix A shows the amino acid sequences of the polypeptides listed in Table 3 (i.e., P1 sequences).

Appendix B shows the nucleotide sequences of the open reading frame (i.e., N1 sequences) of the polypeptides listed in Table 3.

### INDUSTRIAL APPLICABILITY

The polypeptides, polynucleotides and compositions containing such, and methods and kits described herein. These polypeptides, polynucleotides and compositions are useful in the treatment of neurological diseases that involve myelin degeneration, deficiency or loss by promoting myelination or remyelination. They are also useful in one or more of stimulating proliferation of oligodendrocyte precursor cells (OPC), promoting OPC cell growth, and/or promoting OPC cell survival.

### SUMMARY OF THE INVENTION

The present invention provides polypeptides, polynucleotides, and compositions containing such that can be used for repair and/or promoting myelination or remyelination in subjects wherein such repair or treatment is desirable. Exemplary embodiments of the present invention are set forth as follows.

1. An isolated nucleic acid molecule comprising a first polynucleotide sequence of any one of: (A) SEQ. ID. NOS.: 122, 135, 137, 161-162, 176, 192 or 201; (B) a polynucleotide sequence encoding a polypeptide of SEQ. ID. NOS.: 9, 22, 24, 48-49, 63, 79, or 88 (C) biologically active fragments thereof; or (D) complements thereof.

2. The nucleic acid molecule of 1, wherein the polynucleotide sequence comprises a cDNA molecule, a genomic DNA molecule, a cRNA molecule, a siRNA molecule, a RNAi molecule, an mRNA molecule, an antisense molecule, an aptamer, or a ribozyme.

3. A double-stranded isolated nucleic acid molecule comprising the first nucleic acid molecule of 1 and its complement.

4. An isolated polypeptide comprising an amino acid sequence, wherein the amino acid sequence comprises any of SEQ. ID. NOS.: 9, 22, 24, 48-49, 63, 79, or 88.

5. An isolated polypeptide encoded by the first nucleic acid molecule of 1.

6. An isolated polypeptide comprising any mature polypeptide of Table 5.

7. A polypeptide comprising SEQ. ID. NO.:9 or a fragment thereof.

9. A polypeptide comprising SEQ. ID. NO.:22 or a fragment thereof.

10. A polypeptide comprising SEQ. ID. NO.:24 or a fragment thereof.

11. A polypeptide comprising SEQ. ID. NO.:48 or a fragment thereof.

12. A polypeptide comprising SEQ. ID. NO.:49 or a fragment thereof.

13. A polypeptide comprising SEQ. ID. NO.:63 or a fragment thereof.

14. A polypeptide comprising SEQ. ID. NO.:79 or a fragment thereof.

15. A polypeptide comprising SEQ. ID. NO.:88 or a fragment thereof.

16. A polypeptide encoded by a polynucleotide comprising SEQ. ID. NO.:122 or a fragment thereof.

17. A polypeptide encoded by a polynucleotide comprising SEQ. ID. NO.:135 or a fragment thereof.

18. A polypeptide encoded by a polynucleotide comprising SEQ. ID. NO.:137 or a fragment thereof.

19. A polypeptide encoded by a polynucleotide comprising SEQ. ID. NO.:161 or a fragment thereof.

20. A polypeptide encoded by a polynucleotide comprising SEQ. ID. NO.:162 or a fragment thereof.

21. A polypeptide encoded by a polynucleotide comprising SEQ. ID. NO.:176 or a fragment thereof.

22. A polypeptide encoded by a polynucleotide comprising SEQ. ID. NO.:192 or a fragment thereof.

23. A polypeptide encoded by a polynucleotide comprising SEQ. ID. NO.:201 or a fragment thereof.

24. An isolated polypeptide comprising any of a Pfam domain as shown in Table 4.

25. The polypeptide of any of 4-23 wherein the polypeptide lacks a signal sequence.

26. An isolated polypeptide comprising a signal sequence as shown in Table 5 of any of SEQ ID NOS.: 9, 22, 24, 48-49, 63, 79, or 88.

27. The isolated nucleic acid molecule of 1 further comprising a second polynucleotide encoding a fusion partner.

28. The isolated nucleic acid molecule of 27, wherein the fusion partner comprises a polymer, an immunoglobulin molecule, a succinyl group, fetuin A, fetuin B, albumin, a leucine zipper domain, an oligomerization domain, a mannose binding protein, a macrophage scavenger protein, an Fc fragment, or an active fragment of any of these.

29. A vector comprising the nucleic acid molecule of 1 and a promoter that regulates the expression of the nucleic acid molecule.

30. The vector of 29, wherein the promoter is any of one that is naturally contiguous to the nucleic acid molecule and one that is not naturally contiguous to the nucleic acid molecule.

31. The vector of 29, wherein the promoter is any of an inducible promoter, a conditionally-active promoter, a constitutive promoter, or a tissue-specific promoter.

32. A recombinant host cell comprising the isolated nucleic acid of 1, the isolated polypeptide of 4, or the vector of 29.

33. The host cell of 32, wherein the host cell comprises a prokaryotic cell and a eukaryotic cell.

34. The host cell of 33, wherein the host cell is a eukaryotic cell comprising a human cell, a non-human mammalian cell, an insect cell, a fish cell, a plant cell, or a fungal cell.

35. A non-human animal injected with the nucleic acid molecule of 34.

36. A non-human animal genetically modified with the nucleic acid molecule of 1.

37. A non-human animal injected with the polypeptide of 4.

38. A nucleic acid composition comprising the nucleic acid molecule of 1 and a carrier.

39. The nucleic acid composition of 38, wherein the carrier is a pharmaceutically acceptable carrier or an excipient.

40. The composition of 39, wherein the pharmaceutically acceptable carrier or excipient comprises saline, phosphate buffered saline, and a lipid based formulation.

41. A polypeptide composition comprising at least one polypeptide of 4 and a carrier.

42. The polypeptide composition of 41, wherein the carrier is a pharmaceutically acceptable carrier or an excipient.

43. A vector composition comprising the vector of 29 and a carrier.

44. The vector composition of 43, wherein the carrier is a pharmaceutically acceptable carrier or an excipient.

45. A host cell composition comprising the host cell of 32 and a carrier.

46. The host cell composition of 45, wherein the carrier is a pharmaceutically acceptable carrier or an excipient.

47. A method of producing a plurality of recombinant host cells comprising:

(a) providing a host cell that comprises a vector comprising the nucleic acid molecule of 1;

(b) allowing the host cell to proliferate to form the plurality of recombinant host cells.

48. A method of producing a polypeptide comprising:

(a) providing a composition comprising the recombinant host cell of 32; and

(b) culturing the recombinant host cell to produce the polypeptide.

49. A method of producing a polypeptide comprising:

(a) providing the nucleic acid of 1; and

(b) expressing the nucleic acid molecule in an expression system to produce the polypeptide.

50. The polypeptide composition of 41, wherein the polypeptide further comprises a fusion partner.

51. The polypeptide composition of 50, wherein the fusion partner comprises a polymer, an immunoglobulin molecule, a succinyl group, fetuin A, fetuin B, albumin, a leucine zipper domain, an oligomerization domain, a mannose binding protein, a macrophage scavenger protein, an Fc fragment, or an active fragment of any of these.

52. The polypeptide composition of 41, wherein the polypeptide is pegylated.

53. A diagnostic kit comprising a composition comprising a polynucleotide molecule of 1 and a vehicle.

54. A diagnostic kit comprising an antibody that specifically binds to the polypeptide of 4 or a biologically active fragment thereof.

55. A diagnostic kit comprising the polypeptide of 4 or active fragments thereof.

56. A method of determining the presence of the nucleic acid molecule of 1 in a sample comprising:

(a) providing the nucleic acid molecule of 1;

(b) allowing the nucleic acid molecule of 1 to interact with the sample under conditions that allow for specific binding; and

(c) determining whether specific binding has occurred.

57. A method of determining the presence of an antibody specific to the polypeptide of 4 in a sample comprising:

(a) providing a composition comprising the polypeptide of 4;

(b) allowing the polypeptide to interact with the sample under conditions that allow for specific binding; and

(c) determining whether specific binding has occurred between the polypeptide and the antibody.

58. A method of determining the presence of the polypeptide of 4 in a sample comprising:

(a) providing the polypeptide of 4;

(b) allowing the polypeptide of 4 to interact with the sample under conditions that allow for specific binding; and

(c) determining whether specific binding has occurred.

59. A method of treating an autoimmune disease in a subject comprising:

(a) providing a first composition comprising at least one polypeptide from any of 4-24, 29-34, 41-46, and SEQ ID NOS: 1-113, or an active fragment thereof, and a carrier; and

(b) administering the composition to the subject.

60. The method of 59, further comprising a second composition comprising at least one therapeutic agent of any of a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic and an antidiuretic.

61. The method of 60, wherein the interferon comprises any of IFN-beta1B (Betaseron) and IFN-beta1A (e.g., Avonex, Rebif).

62. The method of 59, wherein the autoimmune disease is a type of multiple sclerosis, systemic lupus erythematosus (SLE), Graves' disease, immunoproliferative disease lymphadenopathy (IPL), angioimmunoproliferative lymphadenopathy (AIL), or immunoblastive lymphadenopathy (IBL).

63. The method of 62, wherein the type of multiple sclerosis is any of primary progressive multiple sclerosis, relapsing-remitting multiple sclerosis, secondary-progressive multiple sclerosis, progressive-relapsing multiple sclerosis, or benign multiple sclerosis.

64. The method of 59, wherein administering the composition to the subject comprises administering the composition locally or systemically.

65. The method of 59, wherein administering the composition to the subject comprises administering the composition subcutaneously, intraperitoneally, intravenously, intramuscularly, systemically, intranasally, inhalation, orally, intrathecally, or transdermally.

66. The method of 59, wherein the carrier is a pharmaceutically acceptable carrier or an excipient.

67. The method of 59, wherein the composition is administered in a therapeutically effective amount.

68. The method of 67, wherein the therapeutically effective amount is in a range of about 1 ng/kg to about 100 mg/kg weight of the subject.

69. The method of 68, wherein the therapeutically effective amount is in a range of about 10 ng/kg to about 80 mg/kg weight of the subject.

70. The method of 68, wherein the therapeutically effective amount is in a range of about 50 ng/kg to about 50 mg/kg weight of the subject.

71. The method of 68, wherein the therapeutically effective amount is in a range of about 0.1 ug/kg to about 20 mg/kg weight of the subject.

72. The method of 68, wherein the therapeutically effective amount is in a range of about 0.3 ug/kg to about 10 mg/kg weight of the subject.

73. The method of 68, wherein the therapeutically effective amount is in a range of about 0.5 ug/kg to about 8 mg/kg weight of the subject.

74. The method of 68, wherein the therapeutically effective amount is in a range of about 1.0 ug/kg to about 5 mg/kg weight of the subject.

75. The method of 68, wherein the therapeutically effective amount is in a range of about 5 ug/kg to about 3 mg/kg weight of the subject.

76. The method of 68, wherein the therapeutically effective amount is in a range of about 10 ug/kg to about 1 mg/kg weight of the subject.

77. The method of 68, wherein the therapeutically effective amount is in a range of about 20 ug/kg to about 100 ug/kg weight of the subject.

78. The method of 68, wherein the therapeutically effective amount is in a range of about 50 ug/kg to about 75 ug/kg weight of the subject.

79. A method for treating a demyelinating disease, disorder or condition in a subject comprising:

(a) providing a first composition comprising at least one polypeptide of any of 4-24, 29-34, 41-46, SEQ ID NOS: 1-113, or an active fragment thereof, and a carrier; and

(b) administering the composition to the subject.

80. The method of 79, wherein the demyelinating disease, disorder or condition is any of a type of multiple sclerosis or a disease disorder or condition of Table 6.

81. The method of 80, wherein the type of multiple sclerosis is any of primary progressive multiple sclerosis, relapsing-remitting multiple sclerosis, secondary-progressive multiple sclerosis, progressive-relapsing multiple sclerosis, or benign multiple sclerosis.

82. The method of 79, further comprising a second composition comprising at least one therapeutic agent comprising a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic, or an antidiuretic.

83. The method of 82, wherein the interferon is any of IFN-beta1B (Betaseron) and IFN-beta1A (e.g., Avonex, Rebif).

84. The method of 79, wherein administering the composition to the subject comprises administering the composition locally or systemically.

85. The method of 79, wherein administering the composition to the subject comprises administering the composition subcutaneously, intraperitoneally, intravenously, intramuscularly, systemically, intranasally, inhalation, orally, intrathecally, or transdermally.

86. The method of 79, wherein the carrier is a pharmaceutically acceptable carrier or an excipient.

87. The method of 79, wherein the composition is administered in a therapeutically effective amount.

88. A method of promoting remyelination in a subject comprising administering to the subject a first composition comprising at least one polypeptide of any of 4-24, 29-34, 41-46, SEQ ID NOS: 1-113, or an active fragment thereof, and a carrier.

89. The method of 88, wherein the subject suffers from a demyelinating disease, disorder or condition of Table 6.

90. The method of 88, wherein the subject suffers from a type of multiple sclerosis.

91. The method of 90, wherein the type of multiple sclerosis is any of primary progressive multiple sclerosis, relapsing-remitting multiple sclerosis, secondary-progressive multiple sclerosis, progressive-relapsing multiple sclerosis, or benign multiple sclerosis.

92. The method of 88, wherein the subject suffers from a spinal cord injury.

93. The method of 88, wherein the subject suffers from a loss of oligodendrocytes.

94. The method of 88, wherein the subject suffers from demyelinating lesions.

95. The method of 88, further comprising a second composition comprising at least one therapeutic agent comprising a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic, or antidiuretic.

96. The method of 95, wherein the interferon is any of IFN-beta1B (Betaseron) and IFN-beta1A (e.g., Avonex, Rebif).

97. The method of 88, wherein administering the composition to the subject comprises administering the composition locally or systemically.

98. The method of 88, wherein administering the composition to the subject comprises administering the composition subcutaneously, intraperitoneally, intravenously, intramuscularly, systemically, intranasally, inhalation, orally, intrathecally, or transdermally.

99. The method of 88, wherein the carrier is a pharmaceutically acceptable carrier or an excipient.

100. The method of 88, wherein the composition is administered in a therapeutically effective amount.

101. A method of treating an inflammatory disease in a subject comprising:

(a) providing a first composition comprising at least one polypeptide of any of 4-24, 29-34, 41-46, SEQ ID NOS: 1-113, or an active fragment thereof, and a carrier; and

(b) administering the composition to the subject.

102. The method of 101, wherein the inflammatory disease is any of a type of multiple sclerosis or chronic inflammatory demyelinating polyneuropathy.

103. The method of 102, wherein the type of multiple sclerosis is any of primary progressive multiple sclerosis, relapsing-remitting multiple sclerosis, secondary-progressive multiple sclerosis, progressive-relapsing multiple sclerosis, or benign multiple sclerosis.

104. The method of 101, further comprising a second composition comprising at least one therapeutic agent comprising a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic, or an antidiuretic.

105. The method of 104, wherein the interferon is any of IFN-beta1B (Betaseron) and IFN-beta1A (e.g., Avonex, Rebif).

106. The method of 101, wherein administering the composition to the subject comprises administering the composition locally or systemically.

107. The method of 101, wherein administering the composition to the subject comprises administering the composition subcutaneously, intraperitoneally, intravenously, intramuscularly, systemically, intranasally, inhalation, orally, intrathecally, or transdermally.

108. The method of 101, wherein the carrier is a pharmaceutically acceptable carrier or an excipient.

109. The method of 101, wherein the composition is administered in a therapeutically effective amount.

110. A method for stimulating oligodendrocyte precursor cell proliferation in a subject comprising:

(a) providing a first composition comprising at least one polypeptide of any of 4-24, 29-34, 41-46, SEQ ID NOS: 1-113, or an active fragment thereof, and a carrier; and

(b) administering the composition to the subject.

111. The method of 110, further comprising a second composition comprising at least one therapeutic agent comprising a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic, or an antidiuretic.

112. The method of 111, wherein the interferon is any of IFN-beta1B (Betaseron) and IFN-beta1A (e.g., Avonex, Rebif).

113. The method of 110, wherein administering the composition to the subject comprises administering the composition locally or systemically.

114. The method of 110, wherein administering the composition to the subject comprises administering the composition subcutaneously, intraperitoneally, intravenously, intramuscularly, systemically, intranasally, inhalation, orally, intrathecally, or transdermally.

115. The method of 110, wherein the carrier is a pharmaceutically acceptable carrier or an excipient.

116. The method of 110, wherein the composition is administered in a therapeutically effective amount.

117. A method for treating a disease in a subject wherein proliferation of oligodendrocyte precursor cells is desirable comprising:

(a) providing a first composition comprising at least one polypeptide of any of 4-24, 29-34, 41-46, SEQ ID NOS: 1-113, or an active fragment thereof, and a carrier; and

(b) administering the composition to the subject.

118. The method of 117, further comprising a second composition comprising at least one therapeutic agent comprising a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic, or an antidiuretic.

119. The method of 118, wherein the interferon is any of IFN-beta1B (Betaseron) and IFN-beta1A (e.g., Avonex, Rebif).

120. The method of 117, wherein administering the composition to the subject comprises administering the composition locally or systemically.

121. The method of 117, wherein administering the composition to the subject comprises administering the composition subcutaneously, intraperitoneally, intravenously, intramuscularly, systemically, intranasally, inhalation, orally, intrathecally, or transdermally.

122. The method of 117, wherein the carrier is a pharmaceutically acceptable carrier or an excipient.

123. The method of 117, wherein the composition is administered in a therapeutically effective amount.

124. A method for treating a type of multiple sclerosis in a subject comprising:

(a) providing a first composition containing at least one polypeptide of any of 4-24, 29-34, 41-46, SEQ ID NOS: 1-113, or an active fragment thereof, and a carrier; and

(b) administering the composition to the subject.

125. The method of 124, wherein the type of multiple sclerosis is any of primary progressive multiple sclerosis, relapsing-remitting multiple sclerosis, secondary-progressive multiple sclerosis, progressive-relapsing multiple sclerosis, or benign multiple sclerosis.

126. The method of 124, further comprising a second composition comprising at least one therapeutic agent comprising a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic, or an antidiuretic.

127. The method of 126, wherein the interferon is any of IFN-beta1B (Betaseron) and IFN-beta1A (e.g., Avonex, Rebif).

128. The method of 124, wherein administering the composition to the subject comprises administering the composition locally or systemically.

129. The method of 124, wherein administering the composition to the subject comprises administering the composition subcutaneously, intraperitoneally, intravenously, intramuscularly, systemically, intranasally, inhalation, orally, intrathecally, or transdermally.

130. The method of 124, wherein the carrier is a pharmaceutically acceptable carrier or an excipient.

131. The method of 124, wherein the composition is administered in a therapeutically effective amount.

132. A pharmaceutical composition for treating a demyelinating disease in a subject comprising at least one polypeptide and a pharmaceutically acceptable carrier, wherein the polypeptide promotes oligodendrocyte precursor cell synthesis and is any of 4-24, 29-34, 41-46, SEQ ID NOS: 1-113, or an active fragment thereof.

133. The pharmaceutical composition of 132, further comprising at least one therapeutic agent of any of a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic and an antidiuretic.

134. The pharmaceutical composition of 133, wherein the interferon is any of IFN-beta1B (Betaseron) and IFN-beta1A (e.g., Avonex, Rebif).

135. The pharmaceutical composition of 132, wherein administering the composition to the subject comprises administering the composition locally or systemically.

136. The pharmaceutical composition of 132, wherein administering the composition to the subject comprises administering the composition subcutaneously, intraperitoneally, intravenously, intramuscularly, systemically, intranasally, inhalation, orally, intrathecally, or transdermally.

137. The pharmaceutical composition of 132, wherein the composition is administered in a therapeutically effective amount.

138. The pharmaceutical composition of 132, wherein the pharmaceutically acceptable carrier comprises a biodegradable carrier.

139. The pharmaceutical composition of 138, wherein the biodegradable carrier comprises a polysaccharide.

140. The pharmaceutical composition of 139, wherein the polysaccharide comprises hyaluronic acid, dextran, dextran sulfate, chondroitin sulfate, dermatan sulfate, keratan sulfate, heparin, heparin sulfate, and/or alginate.

141. The pharmaceutical composition of 138, wherein the biodegradable carrier comprises a polymer of polylactic acid and polyglycolic acid.

142. The pharmaceutical composition of 132, wherein the composition further comprises a polymer, a polypeptide, a succinyl group, a lipid group, or serum albumin.

143. The pharmaceutical composition of 142, wherein the polymer comprises a polyethylene glycol moiety (PEG).

144. The pharmaceutical composition of 142, wherein the polypeptide comprises at least a portion of an Fc fragment.

145. The pharmaceutical composition of 132, wherein the polypeptide further comprises a polypeptide of any of SEQ ID NOS: 1-113 and promotes oligodendrocyte precursor cell proliferation in an oligodendrocyte proliferation assay.

146. The pharmaceutical composition of 132, wherein the demyelinating disease is any of a type of multiple sclerosis, chronic inflammatory demyelinating polyneuropathy, idiopathic demyelinating polyneuropathy, leukodystrophy, Canavan's disease, optic neuritis, transverse myelitis, progressive multifocal leukoencephalopathy, infection induced leukoencephalopathies, toxin or chemotherapy induced demyelination, Guillain-Barre syndrome, and acute disseminated encephalomyelitis.

147. The pharmaceutical composition of 146, wherein the type of multiple sclerosis is any of primary progressive multiple sclerosis, relapsing-remitting multiple sclerosis, secondary-progressive multiple sclerosis, progressive-relapsing multiple sclerosis, or benign multiple sclerosis.

148. The pharmaceutical composition of 132, wherein the subject is a human.

149. The pharmaceutical composition of 132, wherein the polypeptide further comprises a fusion partner.

150. The polypeptide composition of 149, wherein the fusion partner comprises a polymer, an immunoglobulin molecule, a succinyl group, fetuin A, fetuin B, albumin, a leucine zipper domain, an oligomerization domain, a mannose binding protein, a macrophage scavenger protein, an Fc fragment, or an active fragment of any of these.

151. An isolated antibody that specifically binds to and/or interferes with the activity of an antigen that comprises at least six contiguous amino acid residues chosen from any of SEQ. ID. NOS.: 9, 22, 24, 48-49, 63, 79 or 88.

152. An isolated antibody that specifically binds to and/or interferes with the activity of an antigen that comprises at least six contiguous amino acid residues chosen from any of SEQ. ID. NOS.: 1-8, 10-21, 23, 25-47, 50-62, 64-78, 80-87, or 89-113.

153. The antibody of 151 or 152, wherein the antibody is chosen from a polyclonal antibody, a monoclonal antibody, a single chain antibody, and active fragments of any of these.

154. The active fragment of claim 153, chosen from any of an antigen binding fragment, an Fc fragment, a cdr fragment, a V_{H} fragment, a V_{C} fragment, or a framework fragment.

155. A transfected mesenchymal stem cell (MSC) comprising a plurality of mesenchymal stem cells transfected with at least a first nucleic acid molecule of any of SEQ ID NOS.: 114-226, or a variant or an active fragment thereof, wherein the transfected MSC differentiates into an oligodendrocyte precursor cell.

156. The transfected MSC of 155, further comprising at least a second nucleic acid molecule.

157. The transfected MSC of 156, wherein the second nucleic acid molecule any of SEQ ID NOS.: 114-226 and is different from the first nucleic acid molecule.

158. The method of any of 79, 88, 110, 117, or 124, wherein the composition further comprises a plurality of mesenchymal stem cells, wherein the mesenchymal stem cells comprise at least one polynucleotide of any of SEQ ID NOS.: 114-226.

159. A pharmaceutical composition for treating a disease in a subject wherein the disease is a cancer, comprising at least one modulator, wherein the modulator modulates the activity of a polypeptide, or a nucleic acid molecule encoding such, wherein the polypeptide comprises an amino acid sequence chosen from the Tables, Sequence Listing, Figures, and a biologically active fragment of any of these.

160. The pharmaceutical composition of 159, wherein the polypeptide comprises SEQ ID NO. 9.

161. The pharmaceutical composition of 159, wherein the polypeptide comprises SEQ ID NO. 22.

162. The pharmaceutical composition of 159, wherein the polypeptide comprises SEQ ID NO. 24.

163. The pharmaceutical composition of 159, wherein the polypeptide comprises SEQ ID NO. 48.

164. The pharmaceutical composition of 159, wherein the polypeptide comprises SEQ ID NO. 49.

165. The pharmaceutical composition of 159, wherein the polypeptide comprises SEQ ID NO. 63.

166. The pharmaceutical composition of 159, wherein the polypeptide comprises SEQ ID NO. 79.

167. The pharmaceutical composition of 159, wherein the polypeptide comprises SEQ ID NO. 88.

168. The pharmaceutical composition of any of 159-167, wherein the cancer comprises solid tumor cells.

169. The pharmaceutical composition of 159, wherein the cancer is an oligodendroglioma.

170. The pharmaceutical composition of 159, wherein the cancer is an oligoastrocytoma.

171. The pharmaceutical composition of any of 159-167, wherein the subject is a human.

172. The pharmaceutical composition of any of 159-167, wherein the pharmaceutical composition is adapted to be administered locally or systemically.

173. The pharmaceutical composition of any of 159-167, wherein the composition is adapted to be administered subcutaneously, intramuscularly, intra-articularly, transdermally, by inhalation, intranasally, orally, intraperitoneally or intravenously.

174. The pharmaceutical composition of any of 159-167, wherein the pharmaceutically acceptable carrier comprises a biodegradable carrier.

175. The pharmaceutical composition of any of 159-167, wherein the modulator comprises an antibody, a fusion molecule comprising an extracellular domain of the polypeptide, an RNAi, an antisense molecule, a ribozyme, a peptide aptamer, or a nucleic acid aptamer.

176. The pharmaceutical composition of 175, wherein the modulator comprises an antibody.

177. The pharmaceutical composition of 176, wherein the antibody is a humanized antibody.

178. The pharmaceutical composition of 176, wherein the antibody comprises a monoclonal antibody; a polyclonal antibody; a single chain antibody; a targeting antibody; a neutralizing antibody; a stabilizing antibody; a chimeric antibody; an antigen-binding fragment; a fragment comprising a variable region of a heavy chain or a light chain of an immunoglobulin; a fragment comprising a complementarity determining region or a framework region of an immunoglobulin; and one or more active fragments, analogues, and/or antagonists of one or more of these antibodies.

179. The pharmaceutical composition of 174, wherein the biodegradable carrier comprises a polymer of polylactic acid and polyglycolic acid.

180. An isolated antibody specifically recognizing, binding to, interfering with, and/or otherwise modulating the biological activity of at least one polypeptide or polynucleotide chosen from the Tables, Sequence Listing, Figures, or a biologically active fragment of any of these.

181. The antibody of 180, wherein the antibody comprises an activity of: (a) specifically inhibiting the binding of the polypeptide to a ligand; (b) specifically inhibiting the binding of the polypeptide to a substrate; (c) specifically inhibiting the binding of the polypeptide as a ligand; (d) specifically inhibiting the binding of the polypeptide as a substrate; (e) specifically inhibiting cofactor binding; (f) inhibiting protease activity; (g) modulating ligand/receptor interaction; or (h) modulating enzyme/substrate interaction.

182. The antibody of 180, comprising a monoclonal antibody; a polyclonal antibody; a single chain antibody; a targeting antibody; a neutralizing antibody; a stabilizing antibody; a chimeric antibody; an antigen-binding fragment; a fragment comprising a variable region of a heavy chain or a light chain of an immunoglobulin; a fragment comprising a complementarity determining region or a framework region of an immunoglobulin; and one or more active fragments, analogues, and/or antagonists of one or more of these antibodies.

183. The antibody of 180, comprising a backbone of a molecule with an Ig domain or a T cell receptor backbone; a mammalian antibody; a human antibody; a non-human primate antibody; and one or more active fragments, analogues, and/or antagonists of one or more of these antibodies.

184. The antibody of 182, wherein the antibody comprises a monoclonal antibody.

185. The antibody of 182, wherein the antibody comprises an antigen-binding fragment.

186. The antibody of 182, wherein the antibody is produced in a plant, an animal, or a cell.

187. The antibody of 186, wherein the cell is chosen from a bacterial cell, a fungal cell, a plant cell, an insect cell, and a mammalian cell.

188. The antibody of 186, wherein the cell is chosen from a yeast cell, an Aspergillus cell, an SF9 cell, a High Five cell, a cereal plant cell, a tobacco cell, a tomato cell, a 293 cell, a myeloma cell, a NS0 cell, a PerC6 cell, and a CHO cell.

189. A hybridoma comprising the antibody of 180.

190. A method for inhibiting tumor growth comprising the steps of:

(a) providing a modulator composition comprising a modulator of any of SEQ ID NOS.: 1-113, or an active fragment thereof; and

(b) contacting the tumor with the composition.

191. The method of 190, wherein the tumor comprises human tumor cells.

192. The method of 191, wherein the human tumor cells are solid tumor cells or leukemia.

193. A kit for treating a demyelinating disease, disorder or condition in a subject, comprising the composition of any of 4-24, 29-34, 41-46, 132-150, a device for delivering the composition to the subject, and instructions for administering such.

### DETAILED DESCRIPTION OF THE INVENTION

### Definitions

The terms “polypeptide,” “peptide,” and “protein,” can be used interchangeably, refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. The term also includes conjugated proteins, fusion proteins, including, but not limited to, GST fusion proteins, fusion proteins with a heterologous amino acid sequence, fusion proteins with heterologous and homologous leader sequences, fusion proteins with or without N-terminal methionine residues, pegolyated proteins, and immunologically tagged proteins. Also included in this term are variations of naturally occurring proteins, where such variations are homologous or substantially similar to the naturally occurring protein, as well as corresponding homologs from different species. Variants of polypeptide sequences include insertions, additions, deletions, or substitutions compared with the subject polypeptides. The term also includes peptide aptamers.

The terms “nucleic acid molecule,” “nucleotide,” “polynucleotide,” and “nucleic acid” are used interchangeably herein to refer to polymeric forms of nucleotides of any length. They can include both double- and single-stranded sequences and include, but are not limited to, cDNA from viral, prokaryotic, and eukaryotic sources; mRNA; genomic DNA sequences from viral (e.g. DNA viruses and retroviruses) or prokaryotic sources; RNAi; cRNA; antisense molecules; ribozymes; and synthetic DNA sequences. The term also captures sequences that include any of the known base analogs of DNA and RNA.

As used herein, “myelinating cell,” refers to cells capable of producing myelin in the nervous system of both the central nervous system (CNS) and the peripheral nervous system (PNS). Exemplary myelinating cells are oligodendrocytes responsible for producing myelin in the CNS, and Schwann cells responsible for producing myelin in the PNS.

As used herein, the term “Oligodendrocyte precursor cell” or “OPC” refers to cells that mature and develop into oligodendrocytes. This term also includes myelin producing cells.

As used herein, “remyelinating” and/or “remyelinating agent,” refers to any agent or molecule that promotes or stimulated remyelination and/or reduces myelin degeneration, myelin deficiency or loss.

The term “demyelination” refers to the removal, destruction, or breakdown of myelin in neurological tissue. Demyelination consists of the removal of the myelin sheath, such as that surrounding neurons or neuronal projections (e.g., the axons). This process may be chemical or immunological in both the experimental and pathological states. The molecules described herein effect transient demyelination in order to promote repair and regrowth.

As used herein, “central nervous system or CNS,” should be construed to include brain and/or the spinal cord of a mammal. The term may also include the eye and optic nerve in some instances.

As used herein, the term “disease, disorder or condition of the central nervous system” is meant to refer to a disease, disorder or a condition which is manifested by abnormal structure and/or function of the central nervous system, such as, for example, neurodegenerative disease or primary tumor formation. The term should also be construed to encompass other pathologies in the central nervous system that are a result of infiltration of the central nervous system by cells which do not originate in the central nervous system, for example, metastatic tumor formation in the central nervous system. The term should also be construed to include trauma to the central nervous system induced by direct injury to the tissues of the central nervous system. The term should also include a neurodegenerative disease associated with demyelination of cells of the CNS. An example of such a disease is multiple sclerosis (MS).

The term “modulate” refers to the production, either directly or indirectly, of an increase or a decrease, a stimulation, inhibition, interference, or blockage in a measured activity when compared to a suitable control. A “modulator” of a polypeptide or polynucleotide or an “agent” are terms used interchangeably herein to refer to a substance that affects, for example, increases, decreases, stimulates, inhibits, interferes with, or blocks a measured activity of the polypeptide or polynucleotide, when compared to a suitable control.

As used herein, the term “fragment” refers to a peptide or polypeptide comprising an amino acid sequence of at least 5 contiguous amino acid residues, at least 6 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, or at least contiguous 200 amino acid residues of the amino acid sequence of another polypeptide. In a specific embodiment, a fragment of a polypeptide retains at least one function of the polypeptide.

A “biologically active” entity, or an entity having “biological activity,” is one having structural, regulatory, or biochemical functions of a naturally occurring molecule or any function related to or associated with a metabolic or physiological process. Biologically active polypeptide fragments are those exhibiting activity similar, but not necessarily identical, to an activity of a polypeptide of the present invention. The biological activity can include an improved desired activity, or a decreased undesirable activity. For example, an entity demonstrates biological activity when it participates in a molecular interaction with another molecule, or when it has therapeutic value in alleviating a disease condition, or when it has prophylactic value in inducing an immune response to the molecule, or when it has diagnostic value in determining the presence of the molecule. A biologically active polypeptide or fragment thereof includes one that can participate in a biological reaction, for example, one that can serve as an epitope or immunogen to stimulate an immune response, such as production of antibodies, or that can participate in signal transduction by binding to receptors, proteins, or nucleic acids, activating enzymes or substrates.

A “neurodegenerative disease, disorder or condition,” is a disease or medical condition associated with neuron loss or dysfunction. Examples of neurodegenerative diseases or conditions include neurodegenerative diseases, brain injuries or CNS dysfunctions. Neurodegenerative diseases include, for example, Alzheimer's disease, multiple sclerosis (MS), macular degeneration, glaucoma, diabetic retinopathy, peripheral neuropathy, Huntington's disease, amyotrophic lateral sclerosis, and Parkinson's disease. Brain injuries include, for example, stroke (e.g., hemorrhagic stroke, focal ischemic stroke or global ischemic stroke) and traumatic brain injuries (e.g. injuries caused by a brain surgery or physical accidents). CNS dysfunctions include, for example, depression, epilepsy, neurosis and psychosis.

The term “percent identity,” “percent sequence identity” or “% identity,” “% sequence identity” of an analog or variant with a native factor refers to the percentage of amino acid sequence in the native factor which are also found in the analog or variant when the two sequences are aligned. Percent identity can be determined by any methods or algorithms established in the art, such as LALIGN or BLAST.

A “composition” or “pharmaceutical composition” herein refers to a composition that usually contains an excipient, such as a pharmaceutically acceptable carrier that is conventional in the art and that is suitable for administration into a subject for therapeutic, diagnostic, or prophylactic purposes. It can include a cell culture, in which the polypeptide or polynucleotide is present in the cells and/or in the culture medium. In addition, compositions for topical (e.g., oral mucosa, respiratory mucosa) and/or oral administration can form solutions, suspensions, tablets, pills, capsules, sustained-release formulations, oral rinses, or powders, as known in the art and described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st ed.

A “disease” is a pathological condition, for example, one that can be identified by symptoms or other identifying factors as diverging from a healthy or a normal state. The term “disease” includes disorders, syndromes, conditions, and injuries. Diseases include, but are not limited to, demyelinating, proliferative, inflammatory, immune, metabolic, and infectious diseases.

The term “promoting” as used herein refers to agents and/or molecules capable of increasing or improving oligodendrocyte proliferation.

A “subject,” “individual” or “patient” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

An “an oligodendrocyte stimulating factor” is a protein or polypeptide, or the polynucleotide that encodes such, that promotes and/or stimulates myelination or remyelination. Such factors include the “OPC hits” as shown in Tables 1-5.

The terms “stimulates oligodendrocyte proliferation,” or “promotes oligodendrocyte proliferation” refers to agents and/or molecules capable of increasing, improving and/or promoting oligodendrocyte formation and/or production. For example, this can be evidenced in assays, as demonstrated by the assays shown in Examples 4 and 5.

An “OPC proliferation assay” is a method of determining factors that induce and/or promote proliferation of oligodendrocytes. This can be conducted, for example, by adding into a cell culture, for a set period of time, a factor of interest, and measuring the ATP content of the cells. An increase in ATP signal, as measured by luminescence, is indicative of an increase in cell number. See. for example, Examples 4 and 5.

A “complement” of a nucleic acid molecule is a one that is comprised of its complementary base pairs. Deoxyribonucleotides with the base adenine are complementary to those with the base thymidine, and deoxyribonucleotides with the base thymidine are complementary to those with the base adenine. Deoxyribonucleotides with the base cytosine are complementary to those with the base guanine, and deoxyribonucleotides with the base guanine are complementary to those with the base cytosine. Ribonucleotides with the base adenine are complementary to those with the base uracil, and deoxyribonucleotides with the base uracil are complementary to those with the base adenine. Ribonucleotides with the base cytosine are complementary to those with the base guanine, and deoxyribonucleotides with the base guanine are complementary to those with the base cytosine.

The term “host cell” includes an individual cell, cell line, cell culture, or cell in vivo, which can be or has been a recipient of any polynucleotides or polypeptides of the invention, for example, a recombinant vector, an isolated polynucleotide, an antibody or a fusion protein. Host cells include progeny of a single host cell, and the progeny may not necessarily be completely identical (in morphology, physiology, or in total DNA, RNA, or polypeptide complement) to the original parent cell due to natural, accidental, or deliberate mutation and/or change. Host cells can be prokaryotic or eukaryotic, including mammalian, insect, amphibian, reptilian, crustacean, avian, fish, plant, and fungal cells. A host cell includes cells transformed, transfected, transduced, or infected in vivo or in vitro with a polynucleotide of the invention, for example, a recombinant vector. A host cell which comprises a recombinant vector of the invention may be called a “recombinant host cell.

“Secretory leader,” “signal sequence,” or a “leader sequence,” are used interchangeably herein to refer to a sequence of amino acid residues, typically positioned at the N terminus of a polypeptide, which directs the intracellular trafficking of a polypeptide. Polypeptides that contain a secretory leader, signal sequence, or leader sequence typically also contain a secretory leader, signal sequence, or leader sequence cleavage site. Such polypeptides, after cleavage at the cleavage sites, generate mature polypeptides, for example, after extracellular secretion or after being directed to an appropriate intracellular compartment.

An “isolated” or “substantially isolated” polynucleotide, or a polynucleotide in “substantially pure form,” in “substantially purified form,” or as an “isolate,” is one that is substantially free of the sequences with which it is associated in nature, or other nucleic acid sequences that do not include a sequence or fragment of the subject polynucleotides. By substantially free is meant that less than about 90%, less than about 80%, less than about 70%, less than about 60%, or less than about 50% of the composition is made up of materials other than the isolated polynucleotide. For example, where at least about 99% of the total macromolecules is the isolated polynucleotide, the polynucleotide is at least about 99% pure, and the composition comprises less than about 1% contaminant.

The term “receptor” refers to a polypeptide that binds to a specific ligand. The ligand is usually an extracellular molecule which, upon binding to the receptor, usually initiates a cellular response, such as initiation of a signal transduction pathway. A “soluble receptor” is a receptor that lacks a membrane anchor domain, such as a transmembrane domain. A “soluble receptor” may include naturally occurring splice variants of a wild-type transmembrane protein receptor in which the transmembrane domain is spliced out. A “soluble receptor” may include the extracellular domain or any fragment of the extracellular domain of a transmembrane protein receptor. Soluble receptors can modulate a target protein. They can, for example, compete with wild-type receptors for ligand binding and participate in ligand/receptor interactions, thus modulating the activity of or the number of the receptors and/or the cellular activity downstream from the receptors. This modulation may trigger intracellular responses, for example, signal transduction events which activate cells, signal transduction events which inhibit cells, or events that modulate cellular growth, proliferation, differentiation, and/or death, or induce the production of other factors that, in turn, mediate such activities.

The term “antibody” or “immunoglobulin” refers to a protein, e.g., one generated by the immune system, synthetically, or recombinantly, that is capable of recognizing and binding to a specific antigen; antibodies are commonly known in the art. The term includes active fragments, including for example, an antigen binding fragment of an immunoglobulin, a variable and/or constant region of a heavy chain, a variable and/or constant region of a light chain, a complementarity determining region (cdr), and a framework region. The terms include polyclonal and monoclonal antibody preparations, as well as preparations including hybrid antibodies, altered antibodies, chimeric antibodies, hybrid (chimeric) antibody molecules, F(ab′)₂ and F(ab) fragments; Fv molecules (e.g., noncovalent heterodimers), dimeric and trimeric antibody fragment constructs; minibodies, humanized antibody molecules and any functional fragments obtained from such molecules, wherein such fragments retain specific binding.

The terms “treat,” “treating,” and “treatment” and the like are used herein to generally mean obtaining a desired pharmacological and physiological effect. More specifically, the molecules described herein which are used to treat a subject with or diagnosed with a demyelinating disease, disorder, or condition to do one or more of the following: (1) prevent demyelination; (2) inhibit demyelination; (3) promote remyelination; and (4) slow, halt, reduce/reverse a paralysis. Thus, the effect may be prophylactic in terms of preventing or partially preventing a disease, symptom or condition thereof and/or may be therapeutic in terms of a partial or complete cure of a disease, condition, symptom or adverse effect attributed to the disease depending on the condition or disease being treated. The term “treatment”, as used herein, covers any treatment of a disease in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease and/or its symptoms or conditions.

In the context of cancer, the term “treating” includes, for example, any or all of: preventing progression from pre-malignancy to malignancy, preventing growth of tumor cells or cancer cells, preventing replication of tumor cells or cancer cells, lessening of overall tumor burden and ameliorating one or more symptoms associated with the disease.

A “biodegradable carrier” comprises a composition that can be broken down and absorbed in an animal, such as a human. An example of a biodegradable carrier is polylactic acid and polyglycolic acid homo- or hetero-polymers.

A “variant” of a protein includes both naturally occurring and artificially produced polypeptide, for example, genetically engineered proteins, that differ from the wild-type protein. Differences from the wild-type protein may include, but are not limited to, single or multiple amino acid substitutions, truncations, deletions, insertions, and repetitions. The amino acid substitutions can be conservative or non-conservative.

A “fusion molecule” is a molecule, for example, a polynucleotide, polypeptide, or other polymer, that contains all or portions of more than one gene linked together as one molecule. For example, a fusion protein can be produced from splicing strands of recombinant DNA and expressing the hybrid gene. A fusion molecule can be made by genetic engineering, for example, by removing the stop codon from the DNA sequence of the first protein, then appending the DNA sequence of the second protein in-frame. That DNA sequence will then be expressed by a cell as a single protein. Typically this is accomplished by cloning a cDNA into an expression vector in frame with an existing gene.

A “fusion partner” is a molecule that is linked to a polypeptide or polynucleotide, such as one having therapeutic or prophylactic value. A fusion partner can also be a polynucleotide, or polypeptide, or other polymer. For example, a polypeptide can be fused in-frame at the N-terminus and/or C-terminus of, or internally to, a therapeutic or prophylactic polypeptide. For example, the fusion partner may be albumin, any variant of albumin, or any fragment thereof. Another fusion partner may be any variant of fetuin, or any fragment thereof. Yet another fusion partner may be the Fc domain of an immunoglobulin or a variant thereof. See, e.g., U.S. Pat. Nos. 5,116,964; 5,225,538; 5,428,130; 5,455,165; 5,514,582; 5,714,147; and 6,406,697.

The terms “agent,” “substance,” “modulator,” and “compound” are used interchangeably herein. These terms refer to a substance that binds to and/or modulates a level or activity of a polypeptide, or a level of mRNA encoding a polypeptide, or nucleic acid, or that modulates the activity of a cell containing a polypeptide or nucleic acid. These terms also encompass an active substance that can be used to treat disease, disorders, or conditions of the CNS that are associated with demyelination, such as multiple sclerosis.

A “therapeutic agent” refers to an agent or modality that is useful for treatment of a disease, disorder, or condition of the CNS, including any one or more of a biologically active molecule of Tables 1-5.

“Prophylaxis,” as used herein, includes preventing a disease from occurring or recurring in a subject that may be predisposed to the disease but is not currently symptomatic. Treatment and prophylaxis can be administered to an organism, or to a cell in vivo, in vitro, or ex vivo, and the cell subsequently administered to the subject.

A “biological sample,” “patient sample,” “clinical sample” “sample,” or “biological specimen,” are used interchangeably herein, encompasses a variety of sample types obtained from an individual, including biological fluids such as blood, serum, plasma, urine, cerebrospinal fluid, tears, saliva, lymph, dialysis fluid, lavage fluid, semen, and other liquid samples or tissues of biological origin. It includes tissue samples and tissue cultures or cells derived therefrom and the progeny thereof, including cells in culture, cell supernatants, and cell lysates. It includes organ or tissue culture derived fluids, tissue biopsy samples, tumor biopsy samples, stool samples, and fluids extracted from physiological tissues. Cells dissociated from solid tissues, tissue sections, and cell lysates are included. The definition also includes samples that have been manipulated in any way after their procurement, such as by treatment with reagents, solubilization, or enrichment for certain components, such as polynucleotides or polypeptides. Also included in the term are derivatives and fractions of biological samples. A biological sample can be used in a diagnostic, monitoring, or screening assay.

A “therapeutically effective amount” refers to a dose of a therapeutic agent capable of treating a particular disease, disorder or condition, for example, MS, and/or diseases involving demyelination. A therapeutically effective amount may be effective upon the first administration or it may require more than one administration to achieve a desired therapeutic effect.

An “Fc molecule” refers to that part of a heavy chain of an immunoglobulin molecule that does not bind to a light chain and does not contain an antigen binding site. It may be in a monomeric or a dimeric form and may be fused to another molecule, such as one of the anabolic hits herein, facilitating the formation of a dimerized fusion molecule.

A “pharmaceutically acceptable carrier” refers to a non-toxic solid, semisolid or liquid filler, diluent, encapsulating material or formulation auxiliary of any conventional type. A “pharmaceutically acceptable carrier” is non-toxic to recipients at the dosages and concentrations employed, and is compatible with other ingredients of the formulation. For example, the carrier for a formulation containing polypeptides suitably does not include oxidizing agents and other compounds that are known to be deleterious to polypeptides. Suitable carriers include, but are not limited to, water, dextrose, glycerol, saline, ethanol, and combinations thereof. The carrier may contain additional agents such as wetting or emulsifying agents, pH buffering agents, or adjuvants which enhance the effectiveness of the formulation. Topical carriers include liquid petroleum, isopropyl palmitate, polyethylene glycol, ethanol (95%), polyoxyethylene monolaurate (5%) in water, or sodium lauryl sulfate (5%) in water. Other materials such as anti-oxidants, humectants, viscosity stabilizers, and similar agents may be added as necessary. Percutaneous penetration enhancers such as Azone may also be included.

A “device” for delivery of the compositions of the present invention is any conventional means appropriate for the mode of delivery intended. For example, if the composition is to be injected, the device includes a needle or needle less syringe or a catheter; if the composition is to be delivered transdermally, the device includes a transdermal patch; if the composition is to be implanted, the device includes a biodegradable or non-biodegradable matrix for holding the composition.

“Injection” is the introduction of a substance into the body. Injection may introduce substances into muscular tissue; subcutaneous tissue; a vascular lumen, for example a vein or artery; synovium or articular joint; or other cavities or canals of the body, for example. The term “injection” includes the use of any suitable device to effect the introduction. The term includes, for example, introduction by catheter. The term also includes, for example, the direct injection of a substance to the joint area.

The terms “subject,” “host,” “individual,” “animal,” and “patient,” used interchangeably herein, refer to mammals, including humans, and also include, but are not limited to, murines, simians, felines, canines, equines, bovines, porcines, ovines, caprines, rabbits, mammalian farm animals, mammalian sport animals, and mammalian pets. In many embodiments, the subjects will be humans. Animal models are of interest for experimental investigations, providing a model for treatment of human disease.

Described herein are a number of novel polypeptides, polynucleotides and compositions and methods containing such. The nucleotide and polypeptide molecules, compositions and methods are useful in treating diseases associated with the CNS and, in particular, diseases, disorders and conditions that involve demyelination. The molecules of the invention were identified by employing an in vitro cell-based assay capable of detecting and measuring cellular proliferation, specifically, oligodendrocyte precursor cell proliferation.

It is to be understood that the following detailed description are exemplary and explanatory only and are not restrictive of the invention, as claimed. Moreover, it must be understood that the invention is not limited to the particular embodiments described, as such may, of course, vary. Further, the terminology used to describe particular embodiments is not intended to be limiting, since the scope of the present invention will be limited only by its claims.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

All publications cited are incorporated by reference herein in their entireties, including references cited in such publications are also incorporated by reference in their entireties.

### Polypeptides

This invention provides novel polypeptides, and related polypeptide compositions. The novel polypeptides of the invention encompass proteins with amino acid sequences as shown in SEQ ID NOS: 9, 22, 24, 48-49, 63, 79, and 88, or active fragments thereof. Such polypeptides are encoded by the nucleic acids, including ones having nucleotide sequences shown in SEQ ID NOS: 122, 135, 137, 161-162, 176, 192 and 201, respectively. The subject polypeptides are human polypeptides, fragments and derivatives thereof. In particular embodiments, a polypeptide of the invention has an amino acid sequence substantially identical to the sequence of any polypeptide encoded by a polynucleotide sequence shown in SEQ ID NOS: 122, 135, 137, 161-162, 176, 192 and 201, or active fragments thereof. In some embodiments, the novel polypeptides cited above and other polypeptides of the invention described herein, are capable of one or more than one of: 1) stimulating and/or promoting proliferation of OPC; 2) promoting OPC cell growth, including differentiation and trans-differentiation; 3) promoting or increasing OPC cell survival in animals, particularly humans.

The polypeptides of the subject invention have been separated from their naturally occurring environment and are present in a non-naturally occurring environment. In certain embodiments, the polypeptides are present in a composition where they are more concentrated than in their naturally occurring environment. For example, substantially purified polypeptides are provided.

In addition to naturally occurring proteins, polypeptides that vary from naturally occurring forms are also provided. Fusion proteins can comprise a subject polypeptide, or fragment thereof, and a polypeptide other than a subject polypeptide (“the fusion partner”) fused in-frame at the N-terminus and/or C-terminus of the subject polypeptide, or internally to the subject polypeptide.

Suitable fusion partners include, but are not limited to, immunologically detectable proteins (e.g., epitope tags, such as hemagglutinin, FLAG, and c-myc); polypeptides that provide a detectable signal or that serve as detectable markers (e.g., a fluorescent protein, e.g., a green fluorescent protein, a fluorescent protein from an Anthozoan species; β-galactosidase; luciferase; cre recombinase; and the like); polypeptides that provide a catalytic function or induce a cellular response; polypeptides that provide for secretion of the fusion protein from a eukaryotic cell; polypeptides that provide for secretion of the fusion protein from a prokaryotic cell; polypeptides that provide for binding to metal ions (e.g., Hisₙ, where n=3-10, e.g., 6His) and structural proteins. Fusion partners can also be those that are able to stabilize the present polypeptide, such as a polymer, for example, polyethylene glycol (“PEG”) and a fragment of an immunoglobulin, such as the Fc fragment of IgG, IgE, IgA, IgM, and/or IgD. Additional fusion partners include, but are not limited to, a succinyl group, fetuin A, fetuin B, albumin, a leucine zipper domain, an oligomerization domain, a mannose binding protein, a macrophage scavenger protein, or an active fragment of any of these.

Detection methods are chosen based on the detectable fusion partner. For example, where the fusion partner provides an immunologically recognizable epitope, an epitope-specific antibody can be used to quantitatively detect the level of polypeptide. In some embodiments, the fusion partner provides a detectable signal, and in these embodiments, the detection method is chosen based on the type of signal generated by the fusion partner. For example, where the fusion partner is a fluorescent protein, fluorescence is measured.

In some embodiments, the active fragments herein exhibit one or more activities associated with a corresponding naturally occurring polypeptide. Fragments herein are useful for generating antibodies that would also bind specifically to the full-length polypeptide. Fragments are also useful in methods of screening for candidate agents that bind to and/or modulate polypeptide activity. Specific fragments of interest include those with biological activity including the ability to serve as an epitope or immunogen, and those that bind to other proteins or to nucleic acids.

The invention provides polypeptides comprising such fragments, including, e.g., fusion polypeptides comprising a subject polypeptide fragment fused in frame (directly or indirectly) to another protein (the “fusion partner”), such as the signal peptide of one protein being fused to the mature polypeptide of another protein. Such fusion proteins are typically made by linking the encoding polynucleotides together in a vector or cassette. Suitable fusion partners include, but are not limited to, immunologically detectable proteins (e.g., epitope tags, such as hemagglutinin, FLAG, and c-myc); polypeptides that provide a detectable signal or that serve as detectable markers (e.g., a fluorescent protein, e.g., a green fluorescent protein, a fluorescent protein from an Anthozoan species; β-galactosidase; luciferase; cre recombinase); polypeptides that provide a catalytic function or induce a cellular response; polypeptides that provide for secretion of the fusion protein from a eukaryotic cell; polypeptides that provide for secretion of the fusion protein from a prokaryotic cell; polypeptides that provide for binding to metal ions (e.g., Hisₙ, where n=3-10, e.g., 6His) and structural proteins. Fusion partners can also be those that are able to stabilize the present polypeptide, such as a polymer, for example, polyethylene glycol (“PEG”), a fragment of albumin and a fragment of an immunoglobulin, such as the Fc fragment of IgG, IgE, IgA, IgM, and/or IgD. Additional fusion partners include, but are not limited to, a succinyl group, fetuin A, fetuin B, albumin, a leucine zipper domain, an oligomerization domain, a mannose binding protein, a macrophage scavenger protein, or an active fragment of any of these.

### Polypeptide Preparation

Polypeptides of the invention can be obtained from naturally-occurring sources or produced synthetically. The proteins will be derived from biological sources that express the proteins. The subject proteins can also be derived from synthetic means, e.g., by expressing a recombinant gene encoding a protein of interest in a suitable system or host or enhancing endogenous expression. Further, peptides can be synthesized in the laboratory by techniques well known in the art.

In all cases, the product can be recovered by any appropriate means known in the art. For example, convenient protein purification procedures can be employed (e.g., see Guide to Protein Purification, Deuthscher et al., 1990). That is, a lysate can be prepared from the original source, (e.g., a cell expressing endogenous polypeptide, or a cell comprising the expression vector expressing the polypeptide(s)), and purified using HPLC, exclusion chromatography, gel electrophoresis, or affinity chromatography, and the like.

The invention thus also provides methods of producing polypeptides. Briefly, the methods generally involve introducing a nucleic acid construct into a host cell in vitro and culturing the host cell under conditions suitable for expression, then harvesting the polypeptide, either from the culture medium (such as from a yeast cell or mammalian cell culture medium) or from the host cell, (e.g., by disrupting the host cell, such as a E. coli host cell), or both.

Moreover, the invention provides polypeptides, including polypeptide fragments, as targets for therapeutic intervention, including use in screening assays, for identifying agents that modulate polypeptide level and/or activity, and as targets for antibody and small molecule therapeutics, for example, in the treatment of diseases, disorders or conditions of the CNS, and specifically, those associated with demyelination.

### Variant and Mutant Polypeptides

It is to be understood that the therapeutic polypeptides covered by the invention include biologically active fragments and analogs of therapeutic polypeptides. A biologically active fragment or analog, like the corresponding full length and mature polypeptide, as shown in Table 5, is capable of treating diseases, disorders or conditions of the CNS and/or diseases involving the degeneration, destruction, or breakdown of myelin in neurological tissue. Analogs of a particular therapeutic polypeptide can differ from the therapeutic polypeptide by amino acid sequence differences, or by modifications (e.g., post-translational modifications), which do not affect sequence, or by both. Analogs of the invention will generally exhibit at least 80%, at least 85%, at least 90%, or at least 99% amino acid identity with all or part of the amino acid sequence of a therapeutic polypeptide. Methods for assaying the capacity of biologically active fragments and analogs to treat diseases, disorders or conditions of the CNS and/or diseases involving the degeneration, destruction, or breakdown of myelin in neurological tissue.

Protein engineering may be employed to improve or alter the characteristics of the polypeptides of the invention. Recombinant DNA technology known to those skilled in the art can be used to create novel mutant proteins or “muteins” including single or multiple amino acid substitutions, deletions, additions, or fusion proteins. Such modified polypeptides can show desirable properties, such as enhanced activity or increased stability. In addition, they may be purified in higher yields and show better solubility than the corresponding natural polypeptide, at least under certain purification and storage conditions.

### N-Terminal and C-Terminal Deletion Mutants

For instance, for many proteins, including the extracellular domain of a membrane associated protein or the mature form(s) of a secreted protein, it is known in the art that one or more amino acids may be deleted from the N-terminus or C-terminus without substantial loss of biological function. For instance, Ron et al., J. Biol. Chem., 268:2984-2988 (1993), reported modified KGF proteins that had heparin binding activity even if 3, 8, or 27 amino-terminal amino acid residues were missing.

However, even if deletion of one or more amino acids from the N-terminus of a protein results in modification or loss of one or more biological functions of the protein, other biological activities may still be retained. Thus, the ability of the shortened protein to induce and/or bind to antibodies which recognize the complete or mature from of the protein generally will be retained when less than the majority of the residues of the complete or mature protein are removed from the N-terminus. Whether a particular polypeptide lacking N-terminal residues of a complete protein retains such immunologic activities can be determined by routine methods described herein and otherwise known in the art. Accordingly, the present invention further provides polypeptides having one or more residues deleted from the amino terminus of the amino acid sequences of the molecules shown in Tables 1-5 and SEQ ID NOS: 1-113.

Similarly, many examples of biologically functional C-terminal deletion muteins are known. For instance, interferon gamma increases in activity as much as ten fold when 8-10 amino acid residues are deleted from the carboxy terminus of the protein, see, for example, Dobeli et al., J. Biotechnology, 7:199-216 (1988). However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature protein are removed from the C-terminus. Whether a particular polypeptide lacking C-terminal residues of a complete protein retains such immunologic activities can be determined by routine methods described herein and otherwise known in the art.

### Other Mutants

In addition to terminal deletion forms of the protein discussed above, it also will be recognized by one of ordinary skill in the art that some amino acid sequences of the therapeutic polypeptides of the invention can be varied without significant effect of the structure or function of the protein. If such differences in sequence are contemplated, it should be remembered that there will be critical areas on the protein which determine activity.

Thus, the invention further includes variations of the polypeptides of the invention which show substantial biological activity. Such mutants include deletions, insertions, inversions, repeats, and type substitutions, selected according to general rules known in the art, so as have little effect on activity. For example, guidance concerning how to make phenotypically silent amino acid substitutions is provided in Bowie et al., Science, 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections, or screens, to identify sequences that maintain functionality.

These studies report that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, et al., supra, and the references cited therein. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg, and replacements between the aromatic residues Phe and Tyr.

Thus, a fragment, derivative, or analog of a polypeptide of Appendix B or polypeptide encoded by a nucleic acid sequence of Appendix A may be (i) one in which one or more of the amino acid residues are substituted with a conserved or non-conserved amino acid residue; such a substituted amino acid residue may or may not be one encoded by the genetic code; (ii) one in which one or more of the amino acid residues includes a substituent group; (iii) one in which the mature polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide, a leader or secretory sequence, a sequence employed to purify the above form of the polypeptide, or a proprotein sequence. Such fragments, derivatives, and analogs are deemed to be within the scope of those skilled in the art from the teachings herein.

Thus, the polypeptides of the invention may include one or more amino acid substitutions, deletions, or additions, either from natural mutations or human manipulation. As indicated, these changes may be of a minor nature, such as conservative amino acid substitutions, that do not significantly affect the folding or activity of the protein. Conservative amino acid substitutions include the aromatic substitutions Phe, Trp, and Tyr; the hydrophobic substitutions Leu, Iso, and Val; the polar substitutions Glu and Asp; the basic substitutions Arg, Lys, and His; the acidic substitutions Asp and Glu; and the small amino acid substations Ala, Ser, Thr, Met, and Gly.

Amino acids essential for the functions of the polypeptides of the invention can be identified by methods known in the art, such as site-directed mutagenesis or alanine-scanning mutagenesis, see, for example, Cunningham and Wells, Science, 244:1081-1085 (1989). The latter procedure introduces single alanine mutations. The resulting mutant molecules are then tested for biological activity such as receptor binding, or in vitro or in vitro proliferative activity.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because, for example, aggregates can be immunogenic, Pinckard et al., Clin. Exp. Immunol., 2:331-340 (1967); Robbins et al., Diabetes, 36:838-845 (1987); Cleland et al., Crit. Rev. Therapeutic Drug Carrier Systems, 10:307-377 (1993).

Replacing amino acids can also change the selectivity of the binding of a ligand to cell surface receptors. For example, Ostade et al., Nature, 361:266-268 (1993) describes mutations resulting in selective binding of TNF-α to only one of the two known types of TNF receptors. Sites that are critical for ligand-receptor binding can also be determined by structural analysis such as crystallization, nuclear magnetic resonance, or photoaffinity labeling, for example, Smith et al., J. Mol. Biol., 224:899-904 (1992) and de Vos et al., Science, 255:306-312 (1992).

As described herein, a composition of the invention can be administered to the patient through various means, e.g., intravenously and intraperitoneally, and in a variety of formulations, e.g., with or without material that slowly releases the therapeutic agent, with or without matrix material that serves as scaffold. Various materials can be used as matrix material, including, but not limited to, collagen (e.g., rat tail collagen, Roche cat #1 179 179), nanofiber, and alginate. In some embodiments, the therapeutic agent can be administered with or without use of devices such as catheters, and with or without monitoring. The therapeutic compositions can be used to treat patients having a disease, disorder or condition of the CNS and/or diseases involving the degeneration, destruction, or breakdown of myelin in neurological tissue.

### Nucleic Acids

The present invention provides novel polynucleotides encoding the present novel polypeptides and fragments thereof. It provides human polynucleotide sequences and the compositions containing such. The nucleic acids of the subject invention can encode all or a part of the subject proteins. Double or single stranded fragments can be obtained from the DNA sequence by chemically synthesizing oligonucleotides in accordance with conventional methods, for example by restriction enzyme digestion or polymerase chain reaction (PCR) amplification. Use of the polymerase chain reaction are known and other current techniques have been reviewed.

In some embodiment, the present invention includes the novel polynucleotides of SEQ ID NOS: 122, 135, 137, 161-162, 176, 192, or 201. Such polynucleotides are useful for the purposes of stated herein. In further embodiments, a polynucleotide of the invention hybridizes under stringent hybridization conditions to a polynucleotide having the coding region of any one of the sequences shown in SEQ ID NOS: 122, 135, 137, 161-162, 176, 192, or 201, or a complement thereof.

Modifications in the native structure of nucleic acids, including alterations in the backbone, sugars or heterocyclic bases, have been shown to increase intracellular stability and binding affinity and are included in the present invention. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′—NH—S′-O-phosphoroamidate. Peptide nucleic acids replace the entire ribose phosphodiester.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, about 2 kb, and possibly more, of flanking genomic DNA at either the 5′ or 3′ end of the transcribed region. The genomic DNA can be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

Nucleic acid molecules of the invention can comprise heterologous nucleic acid molecules, i.e., nucleic acid molecules other than the subject nucleic acid molecules, of any length. For example, the subject nucleic acid molecules can be flanked on the 5′ and/or 3′ ends by heterologous nucleic acid molecules of from about 1 nucleotide to about 10 nucleotides, from about 10 nucleotides to about 20 nucleotides, from about 20 nucleotides to about 50 nucleotides, from about 50 nucleotides to about 100 nucleotides, from about 100 nucleotides to about 250 nucleotides, from about 250 nucleotides to about 500 nucleotides, or from about 500 nucleotides to about 1000 nucleotides, or more in length.

The subject polynucleotides include those that encode fusion proteins comprising the subject polypeptides fused to “fusion partners.” For example, the present soluble receptor or ligand can be fused to an immunoglobulin fragment, such as an Fc fragment for stability in circulation or to fix complement. Other polypeptide fragments that have equivalent capabilities as the Fc fragments can also be used herein.

The isolated nucleic acids of the invention can be used as probes to detect and characterize gross alteration in a genomic locus, such as deletions, insertions, translocations, and duplications, e.g., applying fluorescence in situ hybridization (FISH) techniques to examine chromosome spreads. The nucleic acids are also useful for detecting smaller genomic alterations, such as deletions, insertions, additions, translocations, and substitutions (e.g., SNPs).

When used as probes to detect nucleic acid molecules capable of hybridizing with nucleic acids described in the Appendices, the nucleic acid molecules can be flanked by heterologous sequences of any length. When used as probes, a subject nucleic acid can include nucleotide analogs that incorporate labels that are directly detectable, such as radiolabels or fluorophores, or nucleotide analogs that incorporate labels that can be visualized in a subsequent reaction, such as biotin or various haptens. Haptens that are commonly conjugated to nucleotides for subsequent labeling include biotin, digoxigenin, and dinitrophenyl.

Suitable fluorescent labels include fluorochromes e.g., fluorescein and its derivatives, e.g., fluorescein isothiocyanate (FITC6-carboxyfluorescein (6-FAM), 2′,7′-dimethoxy-4′,5′-dichloro-6-carboxyfluorescein (JOE),), 6-carboxy-2′,4′,7′,4,7-hexachlorofluorescein (HEX), 5-carboxyfluorescein (5-FAM); coumarin and its derivatives, e.g., 7-amino-4-methylcoumarin, aminocoumarin; bodipy dyes, such as Bodipy FL; cascade blue; Oregon green; rhodamine dyes, e.g., rhodamine, 6-carboxy-X-rhodamine (ROX), Texas red, phycoerythrin, and tetramethylrhodamine; eosins and erythrosins; cyanine dyes, e.g., allophycocyanin, Cy3 and Cy5 or N,N,N′,N′-tetramethyl-6-carboxyrhodamine (TAMRA); macrocyclic chelates of lanthanide ions, e.g., quantum dye, etc; and chemiluminescent molecules, e.g., luciferases.

Fluorescent labels also include a green fluorescent protein (GFP), i.e., a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; other fluorescent mutants of a GFP from Aequoria victoria, e.g., as described in U.S. Pat. Nos. 6,066,476; 6,020,192; 5,985,577; 5,976,796; 5,968,750; 5,968,738; 5,958,713; 5,919,445; 5,874,304; a GFP from another species such as Renilla reniformis, Renilla mulleri, or Ptilosarcus guernyi, as previously described (WO 99/49019; Peelle et al., 2001), “humanized” recombinant GFP (hrGFP) (Stratagene®); any of a variety of fluorescent and colored proteins from Anthozoan species, (e.g., Matz et al., 1999).

Probes can also contain fluorescent analogs, including commercially available fluorescent nucleotide analogs that can readily be incorporated into a subject nucleic acid. These include deoxyribonucleotides and/or ribonucleotide analogs labeled with Cy3, Cy5, Texas Red, Alexa Fluor dyes, rhodamine, cascade blue, or BODIPY, and the like.

Suitable radioactive labels include, e.g., ³²P, ³⁵S, or ³H. For example, probes can contain radiolabeled analogs, including those commonly labeled with ³²P or ³⁵S, such as α-³²P-dATP, -dTTP, -dCTP, and dGTP; γ-³⁵S-GTP and α-³⁵S-dATP, and the like.

The subject nucleic acids include antisense RNA, ribozymes, and RNAi. Further, the nucleic acids of the invention can be used for antisense or RNAi inhibition of transcription or translation using methods known in the art.

### Variant and Mutant Polynucleotides

The present invention further relates to variants of the nucleic acid molecules of the present invention, which encode portions, analogs, or derivatives of the disclosed molecules. Variants may occur naturally, such as a natural allelic variant, i.e., one of several alternate forms of a gene occupying a given chromosomal locus Genes II; Lewin, B., ed., John Wiley & Sons, New York (1985)). Non-naturally occurring variants may be produced using art-known mutagenesis techniques.

Such variants include those produced by nucleotide substitutions, deletions, or additions. The substitutions, deletions, or additions may involve one or more nucleotides. The variants may be altered in coding regions, non-coding regions, or both. Alterations in the coding regions may produce conservative or non-conservative amino acid substitutions, deletions or additions. These may take the form of silent substitutions, additions, or deletions which do not alter the properties or activities of the described polypeptides, or portions thereof.

In an embodiment, the invention provides nucleic acid molecules encoding mature proteins, i.e., those with cleaved signal peptide or leader sequences, e.g., as shown in Table 5. Further embodiments include an isolated nucleic acid molecule comprising a polynucleotide having a nucleotide sequence at least 93% identical, or at least 95%, 96%, 97%, 98% or 99% identical to a polynucleotide from Appendix A, a polypeptide encoded by a polynucleotide shown in Appendix B, or a biologically active fragment of any of these.

A polynucleotide having a nucleotide sequence at least, for example, 95% identical to a reference nucleotide sequence encoding a polypeptide of the invention, is one in which the nucleotide sequence is identical to the reference sequence except that it may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 93%, 95%, 96%, 97%, 98%, or 99% identical to, for instance, the nucleotide sequences set forth in Appendix A can be determined conventionally using known computer programs such as the Bestfit program (Wisconsin Sequence Analysis Package, Version 8 for Unix, Genetics Computer Group, Madison, Wis.). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed. The present application is directed to nucleic acid molecules at least 93%, 95%, 96%, 97%, 98%, or 99% identical to the nucleic acid sequences set forth in Appendix A irrespective of whether they encode a polypeptide capable in one or more of stimulating proliferation of OPC, promoting OPC cell growth, and/or promoting OPC cell survival.

Even where a particular nucleic acid molecule does not have the activity in one or more of stimulating proliferation of OPC, promoting OPC cell growth, and/or promoting OPC cell survival, one of skill in the art would know how to use the nucleic acid molecule, for instance, as a hybridization probe or a polymerase chain reaction (PCR) primer. Uses of the nucleic acid molecules of the present invention that do not encode a polypeptide that promotes oligodendrocyte synthesis include, inter alia, (1) isolating the gene or allelic variants thereof in a cDNA library; (2) in situ hybridization (e.g., “FISH”) to metaphase chromosomal spreads to provide the precise chromosomal location of the particular genes, as described in Verna et al., Human Chromosomes: A Manual of Basic Techniques, Pergamon Press, New York (1988); and Northern blot analysis for detecting expression of the polypeptide in specific tissues.

Of course, due to the degeneracy of the genetic code, one of ordinary skill in the art will immediately recognize that a large number of the nucleic acid molecules having a sequence at least 93%, 95%, 96%, 97%, 98%, or 99% identical to the nucleic acid sequence of the nucleic acid sequences set forth in the Table 3 will encode a polypeptide having activity. In fact, since multiple degenerate variants of these nucleotide sequences encode the same polypeptide, this will be clear to the skilled artisan. It will be further recognized in the art that a reasonable number of nucleic acid molecules that are not degenerate variants will also encode a polypeptide having polypeptide activity, the skilled artisan is fully aware of amino acid substitutions that are either less likely or not likely to significantly affect protein function (e.g., replacing one aliphatic amino acid with a second aliphatic amino acid), as further described below.

### Vectors and Host Cells

The present invention also relates to vectors which include the isolated nucleic acid molecules of the present invention, host cells which are genetically engineered with the recombinant vectors, and the production of the disclosed polypeptides or fragments thereof by recombinant techniques. The vector may be, for example, a phage, plasmid, viral, or retroviral vector. Retroviral vectors may be replication competent or replication defective. In the latter case, viral propagation generally will occur only in complementing host cells.

The present invention provides recombinant vectors that contain, for example, nucleic acid constructs that encode secretory leader sequences and a selected heterologous polypeptide of interest, and host cells that are genetically engineered with the recombinant vectors. Selected heterologous polypeptides of interest in the present invention include, for example, an extracellular fragment of a secreted protein, a type I membrane protein, a type II membrane protein, a multi-membrane protein, and a soluble receptor. These vectors and host cells can be used for the production of polypeptides described herein, including fragments thereof by conventional recombinant techniques. The vector may be, for example, a phage, plasmid, viral or retroviral vector. Retroviral vectors may be replication competent or replication defective. As above, in the latter case, viral propagation generally will occur only in complementing host cells.

The polynucleotides may be joined to a vector containing a secretory leader sequence and a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert can be operatively linked to an appropriate promoter, such as the phage lambda PL promoter; the E. coli lac, trp, phoA and tac promoters; the SV40 early and late promoters; and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs can include a translation initiating codon at the beginning and a termination codon (UAA, UGA, or UAG) appropriately positioned at the end of the polypeptide to be translated.

As indicated, the expression vectors may include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

The selectable markers are genes that confer a phenotype on a cell expressing the marker, so that the cell can be identified under appropriate conditions. Generally, a selectable marker allows the selection of transformed cells based on their ability to thrive in the presence or absence of a chemical or other agent that inhibits an essential cell function. Suitable markers, therefore, include genes coding for proteins which confer drug resistance or sensitivity thereto, impart color to, or change the antigenic characteristics of those cells transfected with a molecule encoding the selectable marker, when the cells are grown in an appropriate selective medium. For example, selectable markers include cytotoxic markers and drug resistance markers, whereby cells are selected by their ability to grow on media containing one or more of the cytotoxins or drugs; auxotrophic markers by which cells are selected for their ability to grow on defined media with or without particular nutrients or supplements, such as thymidine and hypoxanthine; metabolic markers for which cells are selected, e.g., their ability to grow on defined media containing the appropriate sugar as the sole carbon source, and markers which confer the ability of cells to form colored colonies on chromogenic substrates or cause cells to fluoresce.

Among vectors suitable for use in bacteria include pQE70, pQE60, and pQE-9, available from QIAGEN, Inc., supra; pBS vectors, Phagescript vectors, Bluescript vectors, pNH8A, pNH6a, pNH18A, pNH46A, available from Stratagene (La Jolla, Calif.); and ptrc99a, pKK223-3, pKK233-3, pDR540, pRIT5 available from Pharmacia (Peapack, N.J.). Among suitable eukaryotic vectors are pWLNEO, pSV2CAT, pOG44, pXT1, and pSG available from Stratagene; and pSVK3, pBPV, pMSG and pSVL, available from Pharmacia. Other suitable vectors will be readily apparent to the skilled artisan.

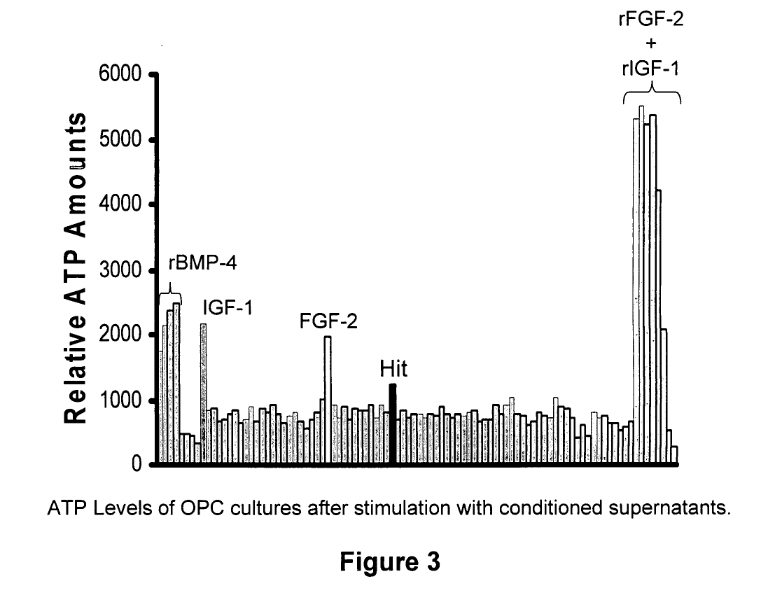

Other suitable vectors include those employing a pTT vector backbone, see, for example, FIGS. 3-7 (Durocher et al. Nucl. Acids Res., 30 (2002)). Briefly, the pTT vector backbone may be prepared by obtaining pIRESpuro/EGFP (pEGFP) and pSEAP basic vector(s), for example from Clontech (Palo Alto, Calif.), and pcDNA3.1, pcDNA3.1/Myc-(His)₆ and pCEP4 vectors can be obtained from, for example, Invitrogen. SuperGlo GFP variant (sgGFP) can be obtained from Q-Biogene (Carlsbad, Calif.). Preparing a pCEP5 vector can be accomplished by removing the CMV promoter and polyadenylation signal of pCEP4 by sequential digestion and self-ligation using SalI and XbaI enzymes resulting in plasmid pCEP4Δ. A GblII fragment from pAdCMV5 (Massie et al., J. Virol., 72: 2289-2296 (1998)), encoding the CMV5-poly(A) expression cassette ligated in BglII-linearized pCEP4A, resulting in pCEP5 vector.

The pTT vector can be prepared by deleting the hygromycin (BsmI and SalI excision followed by fill-in and ligation) and EBNA1 (ClaI and NsiI excision followed by fill-in and ligation) expression cassettes. The ColEI origin (FspI-SalI fragment, including the 3′ end of β-lactamase ORF) can be replaced with a FspI-SalI fragment from pcDNA3.1 containing the pMBI origin (and the same 3′ end of β-lactamase ORF). A Myc-(His)₆ C-terminal fusion tag can be added to SEAP (HindIII-HpaI fragment from pSEAP-basic) following in-frame ligation in pcDNA3.1/Myc-His digested with HindIII and EcoRV.

Plasmids can subsequently be amplified in Escherichia coli (E. coli) (DH5α) grown in LB medium and purified using MAXI prep columns (Qiagen, Mississauga, Ontario, Canada). To quantify, plasmids can be subsequently diluted in 50 mM Tris-HCl pH 7.4 and absorbencies can be measured at 260 nm and 280 nm. Plasmid preparations with A₂₆₀/A₂₈₀ ratios between about 1.75 and about 2.00 are suitable.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986).

As described above, the polypeptides may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide.

The addition of peptide moieties to polypeptides to engender secretion or excretion, to improve stability and to facilitate purification, among others, are familiar and routine techniques in the art. A suitable fusion protein may comprise a heterologous region from immunoglobulin that is useful to stabilize and purify proteins. For example, EP-A-O 464 533 (Canadian counterpart 2045869) discloses fusion proteins containing various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262).

On the other hand, for some uses it would be desirable to be able to delete the Fc part after the fusion protein has been expressed, detected, and purified in the advantageous manner described. This is the case when the Fc portion proves to be a hindrance to use in therapy and diagnosis, for example when the fusion protein is to be used as an antigen for immunizations. In drug discovery, for example, human proteins, such as hIL-5, have been fused with Fc portions for the purpose of high-throughput screening assays to identify antagonists. See, Bennett et al., J. Molec. Recog., 8:52-58 (1995) and Johanson et al, J. Biol. Chem., 270:9459-9471 (1995).

The polypeptides of the invention can be recovered and purified from recombinant cell cultures by well-known methods, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography (HPLC) can be employed for purification.

Polypeptides of the present invention include products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Thus, it is well known in the art that the N-terminal methionine encoded by the translation initiation codon generally is removed with high efficiency from any protein after translation in eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

Typically, a heterologous polypeptide, whether modified or unmodified, may be expressed as described above, or as a fusion protein, and may include not only secretion signals, but also a secretory leader sequence. A secretory leader sequence of the invention directs certain proteins to the endoplasmic reticulum (ER). The ER separates the membrane-bound proteins from other proteins. Once localized to the ER, proteins can be further directed to the Golgi apparatus for distribution to vesicles; including secretory vesicles; the plasma membrane, lysosomes, and other organelles.

Proteins targeted to the ER by a secretory leader sequence can be released into the extracellular space as a secreted protein. For example, vesicles containing secreted proteins can fuse with the cell membrane and release their contents into the extracellular space—a process called exocytosis. Exocytosis can occur constitutively or after receipt of a triggering signal. In the latter case, the proteins may be stored in secretory vesicles (or secretory granules) until exocytosis is triggered. Similarly, proteins residing on the cell membrane can also be secreted into the extracellular space by proteolytic cleavage of a “linker” holding the protein to the membrane.

Additionally, peptide moieties and/or purification tags may be added to the polypeptide to facilitate purification. Such regions may be removed prior to final preparation of the polypeptide. Suitable purification tags include, for example, V5, HISX6, HISX8, avidin, and biotin.

A heterologous polypeptide of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, and high performance liquid chromatography (HPLC).

Polypeptides of the present invention include products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells, or from a cell free expression system. Representative systems from each of these categories are provided below.

Expression systems in plants include those described in U.S. Pat. No. 6,096,546 and U.S. Pat. No. 6,127,145.

Expression systems in bacteria include those described in EP 0 036,776; and U.S. Pat. No. 4,551,433, for example.

Expression systems in yeast include those described in U.S. Pat. Nos. 4,837,148 and 4,929,555; EP 0 244,234; WO 91/00357; and U.S. Pat. No. 6,080,559, for example.

Expression systems for heterologous genes in insects include those described in U.S. Pat. No. 4,745,051; EP 0 127,839; EP 0 155,476; for example.

The insect cell expression system is useful not only for production of heterologous proteins intracellularly, but can be used for expression of transmembrane proteins on the insect cell surfaces. Such insect cells can be used as immunogen for production of antibodies, for example, by injection of the insect cells into mice or rabbits or other suitable animals, for production of antibodies.

Mammalian expression systems include those described, for example, in U.S. Pat. No. 4,399,216. Additional features of mammalian expression are facilitated as described in U.S. Pat. Nos. 4,767,704, 4,657,866, 4,927,762, 4,560,655, WO 90/103430, WO 87/00195, and U.S. Pat. No. RE 30,985, for example. Mammalian cell expression systems can also be used for production of antibodies.

The present polynucleotides can also be used in cell-free expression systems such as bacterial system, e.g., E. coli lysate, rabbit reticulocyte lysate system, wheat germ extract system, frog oocyte lysate system, and the like which is conventional in the art. See, for example, WO 00/68412, WO 01/27260, WO 02/24939, WO 02/38790, WO 91/02076, and WO 91/02075.

Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

When any of the above-referenced host cells, or other appropriate host cells or organisms, are used to replicate and/or express the polynucleotides of the invention, the resulting replicated nucleic acid, RNA, expressed protein or polypeptide, is within the scope of the invention as a product of the host cell or organism.

Once the gene corresponding to a selected polynucleotide is identified, its expression can be regulated in the gene's native cell types. For example, an endogenous gene of a cell can be regulated by an exogenous regulatory sequence inserted into the genome of the cell at a location that will enhance or reduce expression of the gene corresponding to the subject polypeptide. The regulatory sequence can be designed to integrate into the genome via homologous recombination, as disclosed in U.S. Pat. Nos. 5,641,670 and 5,733,761, the disclosures of which are herein incorporated by reference. Alternatively, it can be designed to integrate into the genome via non-homologous recombination, as described in WO 99/15650, the disclosure of which is also herein incorporated by reference. Also encompassed in the subject invention is the production of proteins without manipulating the encoding nucleic acid itself, but rather by integrating a regulatory sequence into the genome of a cell that already includes a gene that encodes the protein of interest; this production method is described in the above-incorporated patent documents.

Antibodies can be made by any method conventional in the art. For example, the antibody can be made in animals, such as in rodents such as mice, or rabbits, or in large animals. The antibody can also be made by phage display, such as by use of the technologies of Cambridge Antibody Technology of Cambridge, U.K., or that of Dyax Corporation (MA, USA), or MorphoSys (Munich, Germany). The antibodies can be made in animals that have been genetically modified to express human antibodies, such as the Xenomouse from Abgenix (Fremont, Calif.) or the Kirin TC Mouse from Kirin Brewery (Japan) or the KM Mouse from Medarex (Princeton, N.J.). The antibodies herein can also be made in cell free wheat germ system, such as by use of the technology of Cell Free Sciences (Japan).

### Epitope-Bearing Portions

As described further below, the polypeptides of the present invention, as shown in Tables 1-5 and SEQ ID NOS.: 1-113, can be used to raise polyclonal and monoclonal antibodies, which are useful in assays for detecting protein expression, or as agonists and/or antagonists capable of enhancing or inhibiting protein function. These polypeptides can also be used in a yeast two-hybrid system to capture binding proteins, which are also candidate agonists and antagonists, according to the present invention. The yeast two hybrid system is described in Fields and Song, Nature, 340:245-246 (1989).

In another aspect, the invention provides a polypeptide comprising one or more epitope-bearing portion of a polypeptide of the invention. An epitope can be an immunogenic epitope or antigenic epitope. Immunogenic epitopes are those parts of a protein that elicit an antibody response when the whole protein is provided as the immunogen. On the other hand, a region of a protein molecule to which an antibody can bind is an antigenic epitope. The number of immunogenic epitopes of a protein generally is less than the number of antigenic epitopes. See, for instance, Geysen et al., Proc. Natl. Acad. Sci., 81:3998-4002 (1983).

As to the selection of polypeptides bearing an antigenic epitope (that is, those which contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe et al., Science, 219:660-666 (1983). Peptides capable of eliciting protein-reactive sera are frequently represented in the primary sequence of a protein, can be characterized by a set of simple chemical rules, and are confined neither to immunodominant regions of intact proteins (that is, to immunogenic epitopes) nor to the amino or carboxyl terminals. Antigenic epitope-bearing peptides and polypeptides of the invention are therefore useful for raising antibodies, including monoclonal antibodies, which bind specifically to a polypeptide of the invention. See, for instance, Wilson et al., Cell, 37:767-778 (1984). The epitope-bearing peptides and polypeptides of the invention may be produced by any conventional means. See, for example, Houghten, Proc. Natl. Acad. Sci. 82:5131-5135 (1985), and U.S. Pat. No. 4,631,211 (1986).

Epitope-bearing peptides and polypeptides of the invention can be used to induce antibodies according to methods well known in the art. See, for instance, Bittle, et al, J. Gen. Virol., 66:2347-2354 (1985). Immunogenic epitope-bearing peptides of the invention, those parts of a protein that elicit an antibody response when the whole protein is the immunogen, are identified according to methods known in the art. See, for instance, U.S. Pat. No. 5,194,392 (1990), which describes a general method of detecting or determining the sequence of monomers (amino acids or other compounds) which is a topological equivalent of the epitope (mimotope) which is complementary to a particular antigen binding site (paratope) of an antibody of interest. More generally, U.S. Pat. No. 4,433,092 (1989) describes a method of detecting or determining a sequence of monomers which is a topographical equivalent of a ligand which is complementary to the ligand binding site of a particular receptor of interest. Similarly, U.S. Pat. No. 5,480,971 (1996) discloses linear C1-C7-alkyl peralkylated oligopeptides, and sets and libraries of such peptides, as well as methods for using such oligopeptide sets and libraries for determining the sequence of a peralkylated oligopeptide that, for example, binds to an acceptor molecule of interest. Thus, non-peptide analogs of the epitope-bearing peptides of the invention also can be made routinely by these methods.

### Antibodies

Antibodies specific to polypeptides of the invention, shown in Tables 1-5 and SEQ ID NOS.: 1-113, are suitable for use in the present invention and can be raised against the intact protein or an antigenic polypeptide fragment thereof. The protein or fragment may be presented with or without a carrier protein, such as an albumin, to an animal, such as a rabbit or mouse). In general, polypeptide fragments are sufficiently immunogenic to produce a satisfactory immune response without a carrier if they are at least about 25 amino acids in length.

Antibodies of the invention include polyclonal and monoclonal antibody preparations, as well as preparations including hybrid antibodies, altered antibodies, chimeric antibodies and, humanized antibodies, as well as hybrid (chimeric) antibody molecules (see, for example, Winter et al., Nature 349:293-299 (1991)); and U.S. Pat. No. 4,816,567); F(ab′)₂ and F(ab) fragments; Fv molecules (noncovalent heterodimers, see, for example, Inbar et al., Proc. Natl. Acad. Sci. 69:2659-2662 (1972)); and Ehrlich et al. (1980) Biochem 19:4091-4096); single chain Fv molecules (sFv) (see, e.g., Huston et al., Proc. Natl. Acad. Sci. 85:5879-5883 (1980)); dimeric and trimeric antibody fragment constructs; minibodies (see, e.g., Pack et al., Biochem. 31:1579-1584 (1992); Cumber et al., J. Immunology 149B:120-126 (1992)); humanized antibody molecules (see, e.g., Riechmann et al., Nature 332:323-327 (1988); Verhoeyan et al., Science 239:1534-1536 (1988)); heteroconjugate and bispecific antibodies (see, e.g., U.S. Pat. No. 6,010,902 and U.S. Patent Appln. 2002/0155604); and any functional fragments obtained from such molecules, wherein such fragments retain specific binding.

Methods of making monoclonal and polyclonal antibodies are known in the art. Monoclonal antibodies are generally antibodies having a homogeneous antibody population. The term is not limited regarding the species or source of the antibody, nor is it intended to be limited by the manner in which it is made. The term encompasses whole immunoglobulins. Polyclonal antibodies are generated by immunizing a suitable animal, such as a mouse, rat, rabbit, sheep or goat, with an antigen of interest, such as a stem cell transformed with a gene encoding an antigen. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as a toxoid from diphtheria, tetanus, cholera, etc., in order to enhance the immunogenicity thereof.

In addition, techniques developed for the production of chimeric antibodies (Morrison et al., Proc. Natl. Acad. Sci., 81:851-855 (1984); Neuberger et al., Nature, 312:604-608 (1984); Takeda et al., Nature, 314:452-454 (1985)) by splicing genes from a mouse antibody molecule of appropriate antigen specificity together with genes from a human antibody molecule of appropriate biological activity can be used. Chimeric antibodies, which are antibodies in which different portions are derived from different animal species, such as those having a variable region derived from a murine monoclonal antibody and a human immunoglobulin constant region, for example, humanized antibodies, and insertion/deletions relating to cdr and framework regions, are suitable for use in the invention.

The invention also includes humanized antibodies, i.e., those with mostly human immunoglobulin sequences. Humanized antibodies of the invention generally refer to non-human immunoglobulins that have been modified to incorporate portions of human sequences. A humanized antibody may include a human antibody that contains entirely human immunoglobulin sequences.

The antibodies of the invention may be prepared by any of a variety of methods. For example, cells expressing the polypeptides of any of SEQ ID NOS.: 1-113 or an antigenic fragment thereof can be administered to an animal in order to induce the production of sera containing polyclonal antibodies. A preparation of any of SEQ ID NOS.: 1-113 can be prepared and purified to render it substantially free of natural contaminants, and the preparation introduced into an animal in order to produce polyclonal antisera with specific binding activity.

Antibodies of the invention specifically bind to their respective antigen(s); they may display high avidity and/or high affinity to a specific polypeptide, or more accurately, to an epitope of an antigen. Antibodies of the invention may bind to one epitope, or to more than one epitope. They may display different affinities and/or avidities to different epitopes on one or more molecules. When an antibody binds more strongly to one epitope than to another, adjusting the binding conditions can, in some instances, result in antibody binding almost exclusively to the specific epitope and not to any other epitopes on the same polypeptide, and not to a polypeptide that does not comprise the epitope.

The invention also provides monoclonal antibodies for any of SEQ ID NOS.: 1-113 or protein binding fragments thereof. Monoclonal antibodies of the invention can be prepared using hybridoma technology, for example, Kohler et al., Nature, 256:495 (1975); Kohler et al., Eur. J. Immunol., 6:511 (1976); Kohler et. al., Eur. J. Immunol., 6:292 (1976); Hammerling et al., in: Monoclonal Antibodies and T-Cell Hybridomas, Elsevier, N.Y., (1981) pp. 563-681. In general, such procedures involve immunizing an animal (for example, a mouse) with any of SEQ ID NOS.: 1-113 protein antigen or protein-expressing cell. Suitable cells can be recognized by their capacity to bind to the selected sequence, i.e., SEQ ID NOS.: 1-113, protein antibody. Such cells may be cultured in any suitable tissue culture medium; for example, in Earle's modified Eagle's medium supplemented with 10% fetal bovine serum (inactivated at about 56° C.), and supplemented with about 10 grams/liter of nonessential amino acids, about 1,000 U/ml of penicillin, and about 100 ug/ml of streptomycin. The splenocytes of such mice are extracted and fused with a suitable myeloma cell line. Any suitable myeloma cell line may be employed in accordance with the present invention; e.g., the parent myeloma cell line (SP20), available from the American Type Culture Collection (ATCC), Manassas, Va. After fusion, the resulting hybridoma cells are selectively maintained in HAT medium, and then cloned by limiting dilution, for example, as described by Wands et al., Gastroenterology, 80:225-232 (1981).

Alternatively, antibodies capable of binding to any of SEQ ID NOS.: 1-113 may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, specific antibodies are used to immunize an animal such as a mouse. The splenocytes of such an animal are then used to produce hybridoma cells, and the hybridoma cells are screened to identify clones which produce an antibody whose ability to bind to the specific antibody can be blocked by the antigen. Such antibodies comprise anti-idiotypic antibodies to any selected sequence of SEQ ID NOS.: 1-113 and can be used to immunize an animal to induce formation of further specific antibodies.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, protein-binding fragments can be produced through the application of recombinant DNA technology or through synthetic chemistry. Humanized chimeric monoclonal antibodies are suitable for in vivo use of the polypeptides in humans. Such humanized antibodies can be produced using genetic constructs derived from hybridoma cells producing the monoclonal antibodies described above. Methods for producing chimeric antibodies are known in the art. See, for review, Morrison, Science, 229:1202 (1985); Oi et al., BioTechniques, 4:214 (1986); Cabilly et al., U.S. Pat. No. 4,816,567; Taniguchi et al., EP 0 171 496; Morrison et al., EP 0 173 494; Neuberger et al., WO 8601533; Robinson et al., WO 8702671; Boulianne et al., Nature, 312:643 (1984); Neuberger et al., Nature, 314:268 (1985).

### Modulators (Agonists and Antagonists)

The invention provides modulators, including polypeptides, polynucleotides, and other agents that increase or decrease the activity of their target. Modulators of the invention may act as an agonist or antagonist, and may interfere with the binding or activity of polypeptides or polynucleotides. Such modulators, or agents, include, for example, polypeptide variants, whether agonist or antagonist; antibodies, whether agonist or antagonist; soluble receptors, usually antagonists; small molecule drugs, whether agonist or antagonist; RNAi, usually an antagonist; antisense molecules, usually an antagonist; and ribozymes, usually an antagonist.

In some embodiments, an agent is a subject polypeptide, where the subject polypeptide itself is administered to an individual. In some embodiments, an agent is an antibody specific for a subject “target” polypeptide. In some embodiments, an agent is a chemical compound, such as a small molecule, that may be useful as an orally available drug. Such modulation includes the recruitment of other molecules that directly effect the modulation. For example, an antibody that modulates the activity of a subject polypeptide that is a receptor on a cell surface may bind to the receptor and fix complement, activating the complement cascade and resulting in lysis of the cell. An agent which modulates a biological activity of a subject polypeptide or polynucleotide increases or decreases the activity or binding at least about 10%, at least about 15%, at least about 20%, at least about 25%, at least about 50%, at least about 80%, or at least about 2-fold, at least about 5-fold, or at least about 10-fold or more when compared to a suitable control.

The invention also provides a method of screening compounds to identify those which modulate the biological activity of a subject polypeptide of the present invention. Examples of the biological activities of the polypeptides of the invention are described in greater detail herein, for example in the Examples and the Figures.

The invention further provides a method wherein a mammalian cell or membrane preparation expressing a receptor for a subject polypeptide of the present invention, as described above, is incubated with a labeled polypeptide in the presence of the compound. The ability of the compound to enhance or block this interaction is then measured. Alternatively, the response of a known second messenger system following interaction of a compound to be screened and a receptor is measured and the ability of the compound to bind to the receptor and elicit a second messenger response is measured to determine if the compound is a potential agonist or antagonist. Such second messenger systems include, but are not limited to, those mediated by cAMP, guanylate cyclase, ion channels, and phosphoinositide hydrolysis.

Examples of antagonistic compounds include antibodies, or in some cases, oligonucleotides, which bind to a receptor of a subject polypeptide but elicit no second messenger response, or which bind to the polypeptide itself. Alternatively, a potential antagonist may be a mutant form of the subject polypeptide which binds to the receptors but elicits no second messenger response, thus effectively blocking the action of the polypeptide.

Another compound antagonistic to a specific gene and/or gene product is an antisense construct prepared using antisense technology. Antisense technology can be used to control gene expression through triple-helix formation or antisense DNA or RNA; both methods are based on the binding of a polynucleotide to DNA or RNA. For example, a 5′ coding portion of the polynucleotide sequence, which encodes mature polypeptides of the present invention, can be used to design an antisense RNA oligonucleotide of from about 10 to about 40 base pairs in length. A DNA oligonucleotide is designed to be complementary to a region of the gene involved in transcription, for example, a triple helix; see Lee et al., Nucl. Acids Res., 6:3073 (1979); Cooney et al., Science, 241:456 (1988); and Dervan et al., Science, 251:1360 (1991); thereby preventing transcription and the production of the polypeptides of the present invention. The antisense RNA oligonucleotide hybridizes to the mRNA in vivo and blocks translation of the mRNA molecule into the polypeptide, as described by Okano, J. Neurochem., 56:560 (1991); Oligodeoxynucleotides as Antisense Inhibitors of Gene Expression, CRC Press, Boca Raton, Fla. (1988). The oligonucleotides described above can also be delivered to cells such that the antisense RNA or DNA is expressed in vivo to inhibit polypeptide production.

The present invention also provides methods for identifying agents, such as antibodies, which enhance or block the actions of the molecules of the invention on cells. For example, these agents may enhance or block interaction of binding molecules, such as receptors. Agents of interest include both agonists and antagonists. The invention provides agonists which increase the natural biological functions of the molecules. The invention also provides antagonists, which decrease or eliminate the functions of the molecules.

One method for identifying suitable agonists and antagonists for a subject molecules involves biochemical assays following subcellular fractionation. Subcellular fractionation methods are known in the art of cell biology, and can be tailored to produce crude fractions with discrete and defined components, for example, organelles or organellar membranes. The preparation is incubated with a labeled subject molecule in the absence or the presence of a candidate molecule which may be an agonist or antagonist to the molecule. The ability of the candidate molecule to interact with the binding molecule is reflected in decreased binding of the labeled ligand. Molecules which bind gratuitously, that is, without inducing the effects of the subject molecule, are most likely antagonists. Molecules that bind well and elicit effects that are the same as or closely related to the subject molecule may potentially prove to be agonists.

The effects of potential agonists and antagonists may by measured, for instance, by determining an activity of one or more components of a second messenger system following interaction of the candidate molecule with a cell or appropriate cell preparation, and comparing the effect with that of a subject molecule. Second messenger systems which may be useful in this regard include, but are not limited to, cAMP, cGMP, ion channels, and phosphoinositide hydrolysis second messenger systems.

Another example of an assay for the identification of an antagonist is a competitive assay that combines a mixture of a subject molecule and a potential antagonist, with membrane-bound receptor molecules. Under appropriate conditions for a competitive inhibition assay, this assay can also be performed with recombinant subject receptor molecules. Subject molecules can be labeled, such as by radioactivity, such that the number of molecules bound to a receptor molecule can be determined accurately to assess the effectiveness of the potential antagonist.

### Diagnostic and Therapeutic Applications

The invention further provides compositions comprising the polypeptides of SEQ ID NOS: 1-113, as shown in Table 3, or active fragments thereof, the mature polypeptides as shown in Table 5, or active fragments thereof, the Pfam domains as shown in Table 4 (indicated by the start and end amino acids), or fragments thereof, the encoding polynucleotides, recombinant vectors containing such, and/or host cells containing such, modulators of such, including pharmaceutical compositions for therapeutic administration. The subject compositions can be formulated using well-known reagents and methods. These compositions can include a carrier or buffer, which is selected according to the desired use of the agent, polypeptide, polynucleotide, recombinant vector, or host cell, and can also include other substances appropriate to the intended use. Those skilled in the art can readily select an appropriate carrier, a wide variety of which are known in the art, suitable for an intended use.

### Excipients and Formulations

In some embodiments, compositions are provided in formulation with pharmaceutically acceptable excipients, a wide variety of which are known in the art Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In pharmaceutical dosage forms, the compositions of the invention can be administered in the form of their pharmaceutically acceptable salts, or they can also be used alone or in appropriate association, as well as in combination, with other pharmaceutically active compounds. The subject compositions are formulated in accordance to the mode of potential administration. Administration of the agents can be achieved in various ways, including oral, buccal, nasal, rectal, parenteral, intraperitoneal, intradermal, transdermal, subcutaneous, intravenous, intra-arterial, intracardiac, intraventricular, intracranial, intratracheal, and intrathecal administration, etc., or otherwise by implantation or inhalation. Thus, the subject compositions can be formulated into preparations in solid, semi-solid, liquid or gaseous forms, such as tablets, capsules, powders, granules, ointments, solutions, suppositories, injections, inhalants and aerosols. The following methods and excipients are merely exemplary and are in no way limiting.

For oral preparations, the agents, polynucleotides, and polypeptides can be used alone or in combination with appropriate additives to make tablets, powders, granules or capsules, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

Suitable excipient vehicles are, for example, water, saline, dextrose, glycerol, ethanol, or the like, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art.

The composition or formulation to be administered will, in any event, contain a quantity of the agent adequate to achieve the desired state in the subject being treated.

The polynucleotides and polypeptides described herein can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Other formulations for oral or parenteral delivery can also be used, as conventional in the art.

The agents, polynucleotides, and polypeptides can be utilized in aerosol formulation to be administered via inhalation. The compounds of the present invention can be formulated into pressurized acceptable propellants such as dichlorodifluoromethane, propane, nitrogen, and the like. Further, the agent, polynucleotides, or polypeptide composition may be converted to powder form for administration intranasally or by inhalation, as conventional in the art.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

A polynucleotide, polypeptide, or modulators thereof, can also be introduced into tissues or host cells by other routes, such as viral infection, microinjection, or vesicle fusion. For example, expression vectors can be used to introduce nucleic acid compositions into a cell as described above. Further, jet injection can be used for intramuscular administration. The DNA can be coated onto gold microparticles, and delivered intradermally by a particle bombardment device, or “gene gun” as described in the literature, where gold microprojectiles are coated with the DNA, then bombarded into skin cells.

Unit dosage forms for oral or rectal administration such as syrups, elixirs, and suspensions can be provided wherein each dosage unit, for example, teaspoonful, tablespoonful, tablet, or suppository, contains a predetermined amount of the composition containing one or more agents. Similarly, unit dosage forms for injection or intravenous administration can comprise the agent(s) in a composition as a solution in sterile water, normal saline or another pharmaceutically acceptable carrier.

Biodegradable carriers can be used to deliver the compositions described herein. In one embodiment, the carrier comprises a cross-linked first and second polysaccharide, as described by U.S. Pat. No. 6,303,585 B1. The first and second polysaccharides are each a derivative of a member selected from the group consisting of hyaluronic acid, dextran, dextran sulfate, chondroitin sulfate, dermatan sulfate, keratan sulfate, heparin, heparin sulfate, and alginate. Aldehyde groups of the first polysaccharide derived from oxidized sugar rings can form covalent imine crosslinks with the second polysaccharide amine derivative at amine sites. The ratios of the first and second polysaccharides determine both the physical and biological properties of the carrier. For example, the ratio can be manipulated to provide unreacted but active aldehydes for covalent linkage to a therapeutic agent, if desired. Advantages of such cross-linked polysaccharide drug carriers include a prolonged bio-degradation rate, controlled release of the therapeutic agent, and flexibility of formulation in gel-like or sponge-like form to accommodate desired therapeutic intervention. Other carriers that can be used in the instant invention include heparin-alginate polymer and alginate as described in Harada et al., J. Clin. Invest. (1994) 94:623-630 and references cited therein.

### Therapeutic Compositions

The present invention provides compositions that are useful in treating diseases, such as neurological diseases, autoimmune diseases, inflammatory diseases, that are characterized and/or associated with myelin degeneration, deficiency or loss. In particular, the invention provides compositions useful in treating MS. The compositions include pharmaceutical compositions, comprising the polypeptides, polynucleotides, and other therapeutic agents.

The compositions may include a carrier, which is selected according to the desired use of the polypeptide, polynucleotide, or other therapeutic agent, and may also include other substances appropriate to the intended use. Those skilled in the art can readily select an appropriate carrier, a wide variety of which are known in the art, suitable for an intended use. The compositions may also include a biodegradable scaffold, matrix or encapsulating material such as liposomes, microspheres, nanospheres and other polymeric substances.

In some instances, the composition can comprise a pharmaceutically acceptable carrier or excipient, a variety of which are known in the art and need not be discussed in detail herein. Pharmaceutically acceptable excipients have been amply described in a variety of publications, including, for example, Gennaro, A. R. (2003) Remington: The Science and Practice of Pharmacy with Facts and Comparisons: Drugfacts Plus. 20^{th} ed., Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed., Amer. Pharmaceutical Assoc. In some embodiments, the composition comprises a matrix that allows for slow release of the composition.

The pharmaceutically acceptable excipients, such as vehicles, adjuvants, carriers, and diluents, are readily available to the public. Moreover, pharmaceutically acceptable auxiliary substances, such as pH adjusting and buffering agents, tonicity adjusting agents, stabilizers, wetting agents and the like, are readily available to the public.

The therapeutic agents may be obtained from naturally occurring sources or synthetically or recombinantly produced. Where obtained from naturally occurring sources, the source chosen will generally depend on the species from which the protein is to be derived. The subject proteins may also be derived by synthesis, such as by synthesizing small fragments of a polypeptide and later linking the small fragments together. The subject protein can be more efficiently produced by recombinant techniques, such as by expressing a recombinant gene encoding the protein of interest in a suitable host, whether prokaryotic or eukaryotic, and culturing such host under conditions suitable to produce the protein.

If a prokaryotic host is selected for production of the protein, such as E. coli, the protein will typically be produced in and purified from the inclusion bodies. If a eukaryotic host is selected for production of the protein, such as CHO or 293 cells, the protein may be secreted into the culture medium when its native or a heterologous secretory leader sequence is linked to the polypeptide to be made. Any convenient protein purification procedures may be employed. Suitable protein purification methodologies are described in Guide to Protein Purification, Deuthser ed. (Academic Press, 1990). For example, a lysate may be prepared from the original source and purified using HPLC, exclusion chromatography, gel electrophoresis, affinity chromatography, and the like.

Therapeutic compositions of the invention may further comprise a second composition comprising one or more agent of a polypeptide, a small organic molecule, a carbohydrate, and or a lipid. These may, in appropriate circumstances, take the form of monomers or polymers. Suitable agents include, for example, a therapeutic agent of any of a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic or an antidiuretic. Specific interferons include, for example, IFN-beta1B (Betaseron) and IFN-beta 1A (e.g., Avonex, Rebif).

The present invention includes an OPC proliferation assay useful in identifying polypeptides having one or more effect of stimulating proliferation of OPC, promoting OPC cell growth, and/or promoting OPC cell survival (Example 5). These polypeptides are useful in treating diseases, disorders, or conditions of the central nervous system (CNS) that are associated with demyelination. These compositions and methods are capable of stimulating and/or promoting myelination or remyelination. The compositions and methods embodied in the present invention are particularly useful as treatments of neurological diseases, such as, multiple sclerosis.

The therapeutic compositions can be formulated into preparations for delivery by dissolving, suspending or emulsifying them in an aqueous solvent, such as phosphate buffered saline (PBS), or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Regarding pharmaceutical dosage forms, the therapeutic compositions may be administered in the form of their pharmaceutically acceptable salts, or they may also be used alone or in appropriate association, as well as in combination, with other pharmaceutically active compounds or treatment procedures. The following methods and excipients are merely exemplary and are in no way limiting.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Gennaro, A. R. (2003) Remington: The Science and Practice of Pharmacy with Facts and Comparisons: Drugfacts Plus. 20^{th} ed., Lippincott, Williams, & Wilkins. The composition or formulation to be administered will, in any event, contain a quantity of the therapeutic agent adequate to achieve the desired state in the subject being treated.

The therapeutic compositions of the invention will be formulated and dosed in a fashion consistent with good medical practice, taking into account the clinical condition of the individual subject, the site of delivery of the polypeptide composition, the method of administration, the scheduling of administration, and other factors known to practitioners. The effective amount of polypeptide for purposes herein is thus determined by such considerations.

### Delivery of Therapeutic Compositions

Further, the present invention provides compositions and methods for treating diseases, disorders or conditions of the CNS and/or diseases involving the degeneration, destruction, or breakdown of myelin in neurological tissue, by administering to a subject at least one composition comprising an effective amount of one or more polypeptides set for in Tables 1-5 and SEQ ID NOS: 1-113, or an active fragment thereof, and a carrier. A second composition may also be optionally administered in combination with a first composition. As described above, the second composition may include, for example, one or more polypeptides as shown in Tables 1-5, an active fragment thereof, or a therapeutic agent such as a corticosteroid, an interferon, such as IFN-beta1B (Betaseron) and IFN-beta1A (e.g., Avonex, Rebif), an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic and an antidiuretic. In accordance with the invention, administration can be effected concomitantly or in sequence.

A therapeutically effective amount of a polypeptide and/or composition of the invention can range from about 1 ng to 80 mg. In one embodiment, the effective amount is an amount about 1 ng/kg (nanogram/kilogram) to about 100 mg/kg (milligram/kilogram) weight of the subject, about 10 ng/kg to about 80 mg/kg weight of the subject, about 50 ng/kg to about 50 mg/kg weight of the subject, about 0.1 ug/kg (microgram/kilogram) to about 20 mg/kg weight of the subject, about 0.3 ug/kg to about 10 mg/kg weight of the subject, about 0.5 ug/kg to about 8 mg/kg weight of the subject, about 1.0 ug/kg to about 5 mg/kg weight of the subject, about 5 ug/kg to about 3 mg/kg weight of the subject, about 10 ug/kg to about 1 mg/kg weight of the subject, about 20 ug/kg to about 100 ug/kg weight of the subject, about 50 ug/kg to about 75 ug/kg weight of the subject.

In order to calculate the amount of a polypeptide to be administered, those skilled in the art could use readily available information with respect to the amount of the polypeptide necessary to have the desired effect. The amount of a polypeptide necessary can be calculated from in vitro or in vivo experimentation. The amount of polypeptide will, of course, vary depending upon the particular composition used and the condition of the subject being treated, such as the subject's age, the extent of the subject's disease, the subject's weight and the likelihood of any adverse effect, etc.

Polypeptides, polynucleotides and/or therapeutic agents of the invention therapeutic agent can be delivered in a matrix composition. The matrix material may serve as scaffold. Various materials can be used as matrix material, including, but not limited to, collagen (for example, rat tail collagen, Roche cat #1 179 179), nanofiber, and alginate. In some embodiments, polypeptides, polynucleotides and/or therapeutic agents of the invention can be administered with or without use of devices such as catheters, and with or without monitoring.

To assist in determining the fate and location of a polypeptide, polynucleotide and/or therapeutic agent of the invention within a patient, a biomarker can be co-administered with the composition containing the polypeptide, polynucleotide and/or therapeutic agent. In one embodiment, a composition containing a polypeptide, polynucleotide and/or therapeutic agent includes the biomarker. Biomarkers can be visualized or detected by a variety of methods, including, but not limited to, x-rays, computed tomography (CT), magnetic resonance imaging (MRI), molecular imaging, or nuclear medicine techniques such as positron emission tomography (PET). Biomarkers that can be used in the present invention, and methods of making and using them, are known in the art.

Polypeptides, polynucleotides and/or therapeutic agents of the invention may be delivered once or a plurality of times. The frequency of treatment and amount of therapeutic agent delivered per treatment will depend on a number of variables, including, but not limited to, the extent and nature of the injury; the potency, toxicity, half-life, solubility, and side effects of the therapeutic agent; and the degree of joint function desired. Those of skill will readily appreciate that dose levels can vary as a function of the specific compound, the severity of the symptoms, and the susceptibility of the subject to side effects. A person of ordinary skill in the art, without undue experimentation, will be able to determine the appropriate frequency and amount of therapeutic agent to use for a particular situation.

The dose may be administered through a variety of routes, including, but not limited to, intravenous, subcutaneous, intramuscular, inhaled, transdermal, etc. Dosing frequency can be once, twice, thrice, once every other month, once every three months, once every six months, once a year, once monthly, once weekly, twice weekly, thrice weekly, every other day, or daily. The dose may be given in one injection, or a plurality of injections, for example, two, three, four, five, six, seven, eight, nine, or ten injections in a given session.

To determine efficacy of the treatment, various parameters may be monitored using a variety of techniques. For example, magnetic resonance imaging may be used.

Polypeptides, polynucleotides and/or therapeutic agents of the invention can be delivered over a period of time by a pump. This delivery may be performed before, simultaneously with, or, or following an acute procedure, such as catheterization, injection, or surgery. The period of time may be in the range of minutes, hours, days, weeks, or months. The pump may be any biocompatible pump, for example, an osmotic pump.

Polypeptides, polynucleotides and/or therapeutic agents of the invention may be delivered alone or in combination with one or more other polypeptide, polynucleotide and/or therapeutic agent of the invention. The exact formulation and combination will depend on a number of factors, including, but not limited to, the extent and nature of the disease, disorder or condition; mode of action of the polypeptide, polynucleotide and/or therapeutic agent; and any interactions between the polypeptide, polynucleotide and/or therapeutic agent. A person of ordinary skill in the art, without undue experimentation, will be able to determine the appropriate combination for a particular situation.

### Therapeutic Fusion Molecules

As one of skill in the art will appreciate, therapeutic polypeptides of the invention can be combined with or joined to heterologous molecules, for example, polypeptides, resulting in chimeric polypeptide molecules. These fusion molecules may facilitate purification. They provide an increased half-life in vivo. This increase has been reported, for example, in chimeric proteins consisting of the first two domains of the human CD4-polypeptide and various domains of the constant regions of the heavy or light chains of mammalian immunoglobulins, for example, EP 0 394 827; Traunecker et al., Nature, 331:84-86 (1988). Fusion proteins with a disulfide-linked dimeric structure due to an immunoglobulin portion can also be more efficient in binding and neutralizing other molecules than the therapeutic protein or protein fragment alone, for example, as described by Fountoulakis et al., J. Biochem., 270:3958-3964 (1995).

Suitable chemical moieties for derivatization of a heterologous polypeptide include, for example, polymers, such as water soluble polymers, succinyl groups, the constant domain of immunoglobulins, all or part of human serum albumin; fetuin A; fetuin B; a leucine zipper domain; a tetranectin trimerization domain; mannose binding protein (also known as mannose binding lectin), for example, mannose binding protein 1; and an Fc fragment, as described herein and further described in U.S. Pat. No. 6,686,179, and U.S. Application Nos. 60/589,788 and 60/654,229. Methods of making fusion proteins are well-known to the skilled artisan.

Polymers, for example, water soluble polymers, are useful in the present invention as the polypeptide to which each polymer is attached will not precipitate in an aqueous environment, such as typically found in a physiological environment. Polymers employed in the invention will be pharmaceutically acceptable for the preparation of a therapeutic product or composition. One skilled in the art will be able to select the desired polymer based on such considerations as whether the polymer/protein conjugate will be used therapeutically and, if so, the desired dosage, circulation time, and resistance to proteolysis.

Suitable, clinically acceptable, water soluble polymers include, but are not limited to, polyethylene glycol (PEG), polyethylene glycol propionaldehyde, copolymers of ethylene glycol/propylene glycol, monomethoxy-polyethylene glycol, carboxymethylcellulose, dextran, polyvinyl alcohol (PVA), polyvinyl pyrrolidone, poly-1,3-dioxolane, poly-1,3,6-trioxane, ethylene/maleic anhydride copolymer, poly (β-amino acids) (either homopolymers or random copolymers), poly(n-vinyl pyrrolidone) polyethylene glycol, polypropylene glycol homopolymers (PPG) and other polyalkylene oxides, polypropylene oxide/ethylene oxide copolymers, polyoxyethylated polyols (POG) (for example, glycerol) and other polyoxyethylated polyols, polyoxyethylated sorbitol, or polyoxyethylated glucose, colonic acids or other carbohydrate polymers, Ficoll, or dextran and mixtures thereof.

As used herein, polyethylene glycol (PEG) is meant to encompass any of the forms that have been used to derivatize other proteins, such as mono-(C1-C10) alkoxy- or aryloxy-polyethylene glycol. Polyethylene glycol propionaldehyde may have advantages in manufacturing due to its stability in water.

Specifically, a modified heterologous polypeptide of the invention may be prepared by attaching polyaminoacids or branch point amino acids to the polypeptide. For example, the polyaminoacid may be a carrier protein that serves to increase the circulation half life of the polypeptide (in addition to the advantages achieved via a fusion molecule). For the therapeutic purpose of the present invention, such polyaminoacids should ideally be those that have or do not create neutralizing antigenic response, or other adverse responses. Such polyaminoacids may be chosen from serum album (such as human serum albumin), an additional antibody or portion thereof, for example the Fc fragment, fetuin A, fetuin B, leucine zipper nuclear factor erythroid derivative-2 (NFE2), neuroretinal leucine zipper, tetranectin, or other polyaminoacids, for example, lysines. As described herein, the location of attachment of the polyaminoacid may be at the N-terminus, or C-terminus, or other places in between, and also may be connected by a chemical linker moiety to the selected molecule.

Polymers used herein, for example water soluble polymers, may be of any molecular weight and may be branched or unbranched. The polymers each typically have an average molecular weight of between about 2 kDa to about 100 kDa (the term “about” indicating that in preparations of a polymer, some molecules will weigh more, some less, than the stated molecular weight). The average molecular weight of each polymer may be between about 5 kDa and about 50 kDa, or between about 12 kDa and about 25 kDa. Generally, the higher the molecular weight or the more branches, the higher the polymer:protein ratio. Other sizes may also be used, depending on the desired therapeutic profile; for example, the duration of sustained release; the effects, if any, on biological activity; the ease in handling; the degree or lack of antigenicity; and other known effects of a polymer on a modified molecule of the invention.

Polymers employed in the present invention are typically attached to a heterologous polypeptide with consideration of effects on functional or antigenic domains of the polypeptide. In general, chemical derivatization may be performed under any suitable condition used to react a protein with an activated polymer molecule. Activating groups which can be used to link the polymer to the active moieties include sulfone, maleimide, sulfhydryl, thiol, triflate, tresylate, azidirine, oxirane, and 5-pyridyl.

Polymers of the invention are typically attached to a heterologous polypeptide at the alpha (α) and/or epsilon (ε) amino groups of amino acids or a reactive thiol group, but it is also contemplated that a polymer group could be attached to any reactive group of the protein that is sufficiently reactive to become attached to a polymer group under suitable reaction conditions. Thus, a polymer may be covalently bound to a heterologous polypeptide via a reactive group, such as a free amino or carboxyl group. The amino acid residues having a free amino group may include lysine residues and the N-terminal amino acid residue. Those having a free carboxyl group may include aspartic acid residues, glutamic acid residues, and the C-terminal amino acid residue. Those having a reactive thiol group include cysteine residues.

Methods for preparing fusion molecules conjugated with polymers, such as water soluble polymers, will each generally involve (a) reacting a heterologous polypeptide with a polymer under conditions whereby the polypeptide becomes attached to one or more polymers and (b) obtaining the reaction product. Reaction conditions for each conjugation may be selected from any of those known in the art or those subsequently developed, but should be selected to avoid or limit exposure to reaction conditions such as temperatures, solvents, and pH levels that would inactivate the protein to be modified. In general, the optimal reaction conditions for the reactions will be determined case-by-case based on known parameters and the desired result. For example, the larger the ratio of polymer:polypeptide conjugate, the greater the percentage of conjugated product. The optimum ratio (in terms of efficiency of reaction in that there is no excess unreacted polypeptide or polymer) may be determined by factors such as the desired degree of derivatization (for example, mono-, di-, tri-, etc.), the molecular weight of the polymer selected, whether the polymer is branched or unbranched and the reaction conditions used. The ratio of polymer (for example, PEG) to a polypeptide will generally range from 1:1 to 100:1. One or more purified conjugates may be prepared from each mixture by standard purification techniques, including among others, dialysis, salting-out, ultrafiltration, ion-exchange chromatography, gel filtration chromatography, and electrophoresis.

One may specifically desire an N-terminal chemically modified protein. One may select a polymer by molecular weight, branching, etc., the proportion of polymers to protein (polypeptide or peptide) molecules in the reaction mix, the type of reaction to be performed, and the method of obtaining the selected N-terminal chemically modified protein. The method of obtaining the N-terminal chemically modified protein preparation (separating this moiety from other monoderivatized moieties if necessary) may be by purification of the N-terminal chemically modified protein material from a population of chemically modified protein molecules.

Selective N-terminal chemical modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved. For example, one may selectively attach a polymer to the N-terminus of the protein by performing the reaction at a pH which allows one to take advantage of the pKa differences between the ε-amino group of the lysine residues and that of the α-amino group of the N-terminal residue of the protein. By such selective derivatization, attachment of a polymer to a protein is controlled: the conjugation with the polymer takes place predominantly at the N-terminus of the protein and no significant modification of other reactive groups, such as the lysine side chain amino groups, occurs. Using reductive alkylation, the polymer may be of the type described above and should have a single reactive aldehyde for coupling to the protein. Polyethylene glycol propionaldehyde, containing a single reactive aldehyde, may also be used.

In one embodiment, the present invention contemplates the chemically derivatized polypeptide to include mono- or poly- (for example, 2-4) PEG moieties. Pegylation may be carried out by any of the pegylation reactions known in the art. Methods for preparing a pegylated protein product will generally include (a) reacting a polypeptide with polyethylene glycol (such as a reactive ester or aldehyde derivative of PEG) under conditions whereby the protein becomes attached to one or more PEG groups; and (b) obtaining the reaction product(s). In general, the optimal reaction conditions for the reactions will be determined case by case based on known parameters and the desired result.

There are a number of PEG attachment methods available to those skilled in the art. See, for example, EP 0 401 384; Malik et al., Exp. Hematol. (1992) 20:1028-1035; Francis, Focus on Growth Factors (1992) 3(2):4-10; EP 0 154 316; EP 0 401 384; WO 92/16221; WO 95/34326; and the other publications cited herein that relate to pegylation.

The step of pegylation as described herein may be carried out via an acylation reaction or an alkylation reaction with a reactive polyethylene glycol molecule. Thus, protein products according to the present invention include pegylated proteins wherein the PEG group(s) is (are) attached via acyl or alkyl groups. Such products may be mono-pegylated or poly-pegylated (for example, those containing 2-6 or 2-5 PEG groups). The PEG groups are generally attached to the protein at the α- or ε-amino groups of amino acids, but it is also contemplated that the PEG groups could be attached to any amino group attached to the protein that is sufficiently reactive to become attached to a PEG group under suitable reaction conditions.

Pegylation by acylation generally involves reacting an active ester derivative of polyethylene glycol (PEG) with a polypeptide of the invention. For acylation reactions, the polymer(s) selected typically have a single reactive ester group. Any known or subsequently discovered reactive PEG molecule may be used to carry out the pegylation reaction. An example of a suitable activated PEG ester is PEG esterified to N-hydroxysuccinimide (NHS). As used herein, acylation is contemplated to include, without limitation, the following types of linkages between the therapeutic protein and a polymer such as PEG: amide, carbamate, urethane, and the like, see for example, Chamow, Bioconjugate Chem. (1994) 5:133-140. Reaction conditions may be selected from any of those known in the pegylation art or those subsequently developed, but should avoid conditions such as temperature, solvent, and pH that would inactivate the polypeptide to be modified.

Pegylation by acylation will generally result in a poly-pegylated protein. The connecting linkage may be an amide. The resulting product may be substantially only (for example, >95%) mono, di- or tri-pegylated. However, some species with higher degrees of pegylation may be formed in amounts depending on the specific reaction conditions used. If desired, more purified pegylated species may be separated from the mixture (particularly unreacted species) by standard purification techniques, including among others, dialysis, salting-out, ultrafiltration, ion-exchange chromatography, gel filtration chromatography and electrophoresis.

Pegylation by alkylation generally involves reacting a terminal aldehyde derivative of PEG with a polypeptide in the presence of a reducing agent. For the reductive alkylation reaction, the polymer(s) selected should have a single reactive aldehyde group. An exemplary reactive PEG aldehyde is polyethylene glycol propionaldehyde, which is water stable, or mono C1-C10 alkoxy or aryloxy derivatives thereof, see for example, U.S. Pat. No. 5,252,714.

Additionally, heterologous polypeptides of the present invention and the epitope-bearing fragments thereof described herein can be combined with or joined to parts of the constant domain of immunoglobulins (IgG), resulting in chimeric polypeptides. These particular fusion molecules facilitate purification and show an increased half-life in vivo. This has been shown, for example, in chimeric proteins consisting of the first two domains of the human CD4-polypeptide and various domains of the constant regions of the heavy or light chains of mammalian immunoglobulins, such as EP 0 394 827; Traunecker et al., Nature (1988) 331:84-86. Fusion molecules that have a disulfide-linked dimeric structure due to the IgG part can also be more efficient in binding and neutralizing other molecules than, for example, a monomeric polypeptide or polypeptide fragment alone; see, for example, Fountoulakis et al., J. Biochem., 270:3958-3964 (1995).

In another described embodiment, a human serum albumin fusion molecule may also be prepared as described herein and as further described in U.S. Pat. No. 6,686,179.

Moreover, the polypeptides of the present invention can be fused to marker sequences, such as a peptide that facilitates purification of the fused polypeptide. The marker amino acid sequence may be a hexa-histidine peptide such as the tag provided in a pQE vector (Qiagen, Mississauga, Ontario, Canada), among others, many of which are commercially available. As described in Gentz et al., Proc. Natl. Acad. Sci. (1989) 86:821-824, for instance, hexa-histidine provides for convenient purification of the fusion protein. Another peptide tag useful for purification, the hemagglutinin HA tag, corresponds to an epitope derived from the influenza hemagglutinin protein (Wilson et al., Cell (1984) 37:767-78). Any of these above fusions can be engineered using the polynucleotides or the polypeptides of the present invention.

### Treatment of Diseases, Disorders and Conditions of the CNS

The present invention includes methods of treating diseases, disorders, or conditions of the central nervous system (CNS) that are associated with demyelination. These compositions and methods are capable of stimulating and/or promoting myelination or remyelination.

CNS diseases, disorders, or conditions include, for example, brain injuries or CNS dysfunctions, Alzheimer's disease, multiple sclerosis (MS), macular degeneration, glaucoma, diabetic retinopathy, peripheral neuropathy, Huntington's disease, amyotrophic lateral sclerosis, and Parkinson's disease. Brain injuries include, for example, stroke (e.g., hemorrhagic stroke, focal ischemic stroke or global ischemic stroke), traumatic brain injuries (e.g. injuries caused by a brain surgery or physical accidents), and spinal cord injury. CNS dysfunctions include, for example, depression, epilepsy, neurosis and psychosis. Other examples include chronic inflammatory demyelinating polyneuropathy, idiopathic demyelinating polyneuropathy, leukodystrophy, Canavan's disease (spongy degeneration), aging, optic neuritis, schizophrenia, transverse myelitis, progressive multifocal leukoencephalopathy, infection induced leukoencephalopathies, a leukodystrophie, toxin or chemotherapy induced demyelination, Guillain-Barre syndrome, adrenoleukodystrophy, Alexander's disease, childhood ataxia with CNS hypomyelination (vanishing-white-matter disease), Krabbe disease (globoid cell leukodystrophy), cerebrotendinous xanthomatosisand acute disseminated encephalomyelitis.

Types of MS include, for example, primary progressive multiple sclerosis, relapsing-remitting multiple sclerosis, secondary-progressive multiple sclerosis, progressive-relapsing multiple sclerosis, or benign multiple sclerosis.

Compositions of the invention may optionally include one or more additional agents. These additional agents can include, for example, a corticosteroid, an interferon, an immunoglobulin, natalizumab (Tysabri), glatiramer acetate (Copaxone), an anticonvulsant, a statin, an inhibitor of CD20 (e.g. Rituximab), methotrexate, mitoxantrone, an analgesic, a mild sedative, a muscle relaxant, a PPAR gamma agonist, Botulinum toxin, an anticholinergic, a urinary tract antispasmodic and an antidiuretic. Interferons include IFN-beta1B (Betaseron) and IFN-beta1A (e.g., Avonex, Rebif).

### Cancer Applications

In another embodiment of the invention, it is desirable to modulate the molecules described herein. As stated above, the molecules described herein are capable of one or more than one of: 1) stimulating and/or promoting proliferation of OPC; 2) promoting OPC cell growth, including differentiation and trans-differentiation; 3) promoting or increasing OPC cell survival in animals, particularly humans. As these molecules have a stimulatory effect on OPC, such activity may also influence the growth properties of malignant tumors in vivo. Thus, it may be desirable to administer desirable to administer a modulator, such as an antagonist, of one or more of these molecules to subjects in need thereof to inhibit undesirable growth and/or proliferation, including those subjects diagnosed and/or afflicted with tumors for treatment of tumors, benign or malignant and at all stages of progression.

For example, malignant tumors that may be treated include primary, recurrent, and/or or metastatic cancerous tumors originating in any tissues, for example, carcinomas, sarcomas, lymphomas, mesotheliomas, melanomas, gliomas, nephroblastomas, glioblastomas, oligodendrogliomas, astrocytomas, oligoastrocytomas, ependymomas, primitive neuroectodermal tumors, atypical meningiomas, malignant meningiomas, or neuroblastomas, originating in the pituitary, hypothalamus, lung, kidney, adrenal, ureter, bladder, urethra, breast, prostate, testis, skull, brain, spine, thorax, peritoneum, ovary, uterus, stomach, liver, bowel, colon, rectum, bone, lymphatic system, skin, or in any other organ or tissue of the subject.

Gliomas include any malignant glial tumor, i.e., a tumor derived from a transformed glial cell. A glial cell includes a cell that has one or more glial-specific features, associated with a glial cell type, including a morphological, physiological and/or immunological feature specific to a glial cell (e.g. astrocytes, oligodendrocytes or microglia), for example, expression of the astroglial marker fibrillary acidic protein (GFAP) or the oligodendroglial marker O4. Gliomas include, but are not limited to, astrocytoma grade II, anaplastic astrocytoma grade III, astrocytoma with oligodendrogliomal component, oligodendroglioma, and glioblastoma multiforme (GBM; astrocytoma grade IV).

Modulators of the invention may be used alone or in combination with therapeutic monoclonal antibodies (for example, Rituxan) to treat cancer,

### Isolation of Human OPC

The isolation of human OPC has been described in the literature, for example, in Ruffini et al., Amer. J. of Path., 165(6):2167-2175 (2004) and Wilson et al., GLIA, 44:153-165 (2003). Briefly, isolation of OPC is achieved from either spinal cord of post mortem samples of adult or embryonic origin, or surgical resections of the brain. After removal of blood vessels and meninges, the tissue is treated with proteases and triturated. Single cells are pelleted and cultured in defined medium, such as Dulbecco's modified eagle medium supplemented with different factors like insulin, PDGF-AA, FGF-2, transferrin, putrescine, thyroxine, tri-iodothyronine, progresterone and sodium selenite in untreated tissue culture flasks. After over-night incubation, the non-attached OPC are harvested and cultured on poly-d-lysine tissue culture plates in defined medium containing PDGF-AA and FGF-2. In order to further enrich the cells a positive selection with, for example, an anti-A2B5 antibody.

### Isolation, Preparation and Delivery of Mesenchymal Stem Cells

A “mesenchymal stem cell” or “MSC” is pluripotent stem cell of mesenchymal origin and that is capable of differentiating into cells of mesenchymal lineages, including muscle, bone, cartilage, fibroblasts, adipose tissue, as well as loose, elastic and fibrous connective tissues. Mesenchyme is an embryonic tissue of mesodermal origin, that is, tissue that derives from the middle of three germ layers. The embryonic mesoderm gives rise to the musculoskeletal, blood, vascular, and urogenital systems, as well as connective tissue. Mesenchymal stem cells can be found in the bone marrow, blood, such as cord blood, dermis and periosteum. Their differentiation pathway, for example, into musculoskeletal, osseous or cartilaginous cells, depends on the agent(s) to which they are exposed.

Mesenchymal stem cells have been shown to also differentiate into neural and glial progenitor cells. Hence, they provide a potential source of myelinating cells (Keirstead, H. S., (2005), Trends in Neurosciences 28(12): 677-683, “Stem cells for treatment of myelin loss”). In some embodiments, the invention provides a population of MSC that are transfected with vectors or plasmids comprising polynucleotides encoding one or more of the molecules described herein. In some embodiments, the invention provides for such MSC expressing the molecules. In some embodiments, the invention provides for methods of using such transfected MSC for treatment of neurological disorders either by replacing endogenous progenitor cells or by providing factors to areas of injury in order to mobilize endogenous repair.

MSC can be obtained from a number of sources conventional in the art, including bone marrow, such as described in Xu, W. et al. (2004), Exp. Biol. Med. 229: 623-631, “Mesenchymal Stem Cells from Adult Human Bone Marrow Differentiate into a Cardiomyocyte Phenotype In Vitro”; cord blood, such as described in Gang, E. J. et al. (2004), Stem Cells 22(4): 617-624, “Skeletal myogenic differentiation of mesenchymal stem cells isolated from human umbilical cord blood” and Lee, O. K. et al. (2004), Blood 103(5): 1669-1675 (Epub 2003 Oct. 23), “Isolation of multipotent mesenchymal stem cells from umbilical cord blood”; adipose tissue, such as described in Tondreau, T. et al. (2005), Stem Cells 23(8): 1105-1112 (Epub. 2005 Jun. 13), “Mesenchymal stem cells derived from CD133-positive cells in mobilized peripheral blood and cord blood: proliferation, Oct4 expression, and plasticity” and Timper, K. et al. (2006), Biochem. Biophys. Res. Commun., 341(4): 1135-1140 (Epub. 2006 Jan. 26), “Human adipose tissue-derived mesenchymal stem cells differentiate into insulin, somatostatin, and glucagon expressing cells.”

MSC can be transfected with the gene encoding one or more of the molecules herein by a number of methods conventional in the art, by use of viral or non-viral vectors. For example, such methods include isolating human mesenchymal stem cells from the bone marrow and transfecting such cells with a viral vector, such as a retroviral vector as described in Lu, Y. et al. (2006), Ann. Clin. Lab. Sci. 36:127-136, “Human Bone Marrow Mesenchymal Stem Cells Transfected with Human Insulin Genes Can Secrete Insulin Stably”; a lentiviral vector as described in Zhang, X. Y. et al. (2004), J. Virol. 78(3): 1219-1229, “Transduction of Bone-Marrow-Derived Mesenchymal Stem Cells by Using Lentivirus Vectors Pseudotyped with Modified RD114 Envelope Glycoproteins”; or an adeno associated viral vector as described in Kim, J. H. et al. (2007), Yonsei Med. J. 48(1): 109-119, “Generation of insulin-producing human mesenchymal stem cells using recombinant adeno-associated virus.”

The MSC transfected with one or more of the molecules herein can be formulated into compositions comprising pharmaceutically acceptable vehicles or carrier. Such formulation may be administered to a subject in need of such treatment, such as neurological disorders, including but not limited to, MS, optic neuritis, spinal cord injury, brain injury, stroke, other demyelination disorders associated with Alzheimer's disease, normal aging and psychiatric disorders such as schizophrenia.

These compositions may be administered in any manner conventional in the art, systemically or locally. For example, the pharmaceutical compositions may be directly applied to any open wounds, such as in spinal cord injuries and traumatic brain injuries, and the like, or during surgery when neuronal tissues are exposed. Further, as examples, such pharmaceutical compositions may be administered to subjects in need of such by implantation or infusion, intracranially, intraventricularly, intravenously, intraspinally or intraperitoneally.

### Kits

The invention further provides a kit comprising a device suitable for use according to the instant invention, for example, in local delivery, including direct injection of a polypeptide, polynucleotide or compositions containing such to treat a disease, disorder or condition of the CNS and/or diseases involving demyelination. The device may be pre-packaged in a sterile container ready for use. The kit may further include additional therapeutic agents and other substances needed to prepare the final composition to be used to treat a disease, disorder or condition of the CNS and/or diseases involving demyelination. In an embodiment, the kit includes unit doses of the therapeutic agent in injectable form. Unit dosage forms for injection may comprise the therapeutic agent in a composition as a solution in sterile water, normal saline, or another pharmaceutically acceptable carrier. In an embodiment, the kit includes unit doses of a therapeutic agent for treating a disease, disorder or condition of the CNS and/or diseases involving demyelination in a patient, for example, any one or more of polypeptides of Tables 1-5; and/or biologically active fragments or variants thereof. In an embodiment, the kit includes instructions for its use. These instructions may describe the attendant benefits of the therapeutic agent in treating a disease, disorder or condition of the CNS and/or diseases involving demyelination and may be provided in a variety of forms. Suitable forms include printed information, a compact disc, and the like. Suitable devices, including catheters; therapeutic agents; and unit doses are those described herein.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications can be made to adapt a particular situation, material, composition of matter, process, process step or steps, to the objective, spirit and scope of the present invention. All such modifications are intended to be within the scope of the claims appended hereto.

### Example 1

### Isolation of Oligodendrocyte Precursor Cells

Oligodendrocyte precursor cells (OPC) were obtained from Dr. Robert H. Miller, Case Western Reserve University (Cleveland, Ohio). Briefly, the cells were isolated from the spinal cord of one entire litter of rat pubs (age P1) from a pregnant dam. The dissociated cells were pan-purified with an antibody directed against the A2B5 cell surface marker to enrich oligodendrocyte precursors. This isolation procedure was performed according to methods described in, for example, Robinson et al., J. Neurosci., 18(24):10457-10463 (1998) and Robinson et al., Mol. Cell. Neurosci., 8(1):38-52 (1996).

### Example 2

### Culturing Primary Rat OPC

Primary rat OPC, according to Example 1, were cultured for 11 days in a prepared culture medium of DMEM (Invitrogen, Carlsbad, Calif.) containing 1% FBS (Mediatech, Herndon, Va.) and N2 supplement (Invitrogen, Carlsbad, Calif.) and FGF-2 and PDGF-β (each at 10 ng/ml; R & D Systems, Minneapolis, Minn.) to support expansion of the cells in a poly-D-lysine (Sigma, St. Louis, Mo.) coated tissue culture flask. Cells were subsequently incubated at 37° C. in a 5% CO₂ incubator (Thermo Fisher Scientific, Waltham, Mass.) and passaged once via mechanical dislodging prior to the high-throughput assay. 50% of the medium was replaced every 3^{rd} day with fresh culture medium. No proteases or other reagent materials were used other than the prepared culture medium for the passage of the cells to ensure surface receptor integrity and maximum viability of the cells.

### Example 3

### Phenotypic Characterization of Passage 1 (P1) OPC

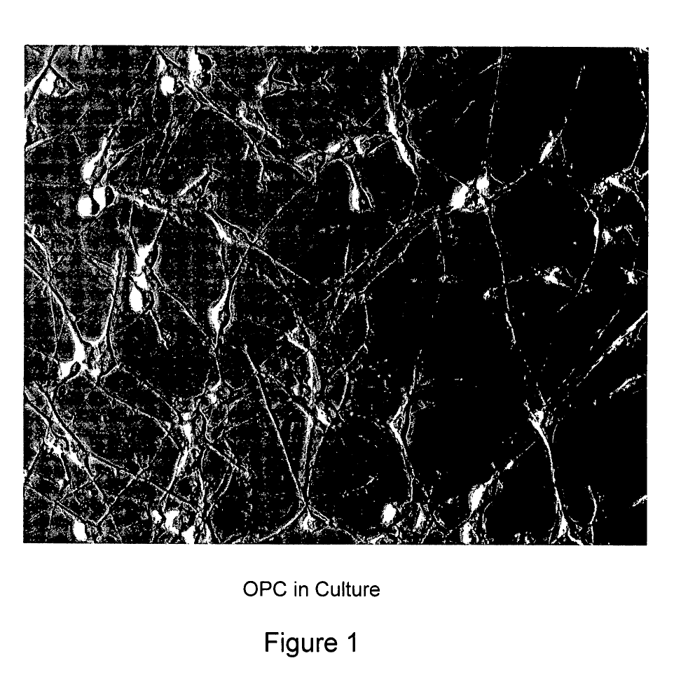

Phenotypic characterization of passage 1 (P1) OPC was accomplished by two different characterization methods to ensure the presence of OPC. The first method was by microscopic analysis of cell morphology. In this method, typically >90% of the cells showed a bi- or tri-polar phenotype when several microscopic fields were analyzed (FIG. 1). The second parameter was the size of the cell body, which is typically approximately 5 μm in diameter. The size of the cell bodies was estimated by microscopy by comparison to a defined length standard.

### Example 4

### Development of High Throughput OPC Proliferation Assay

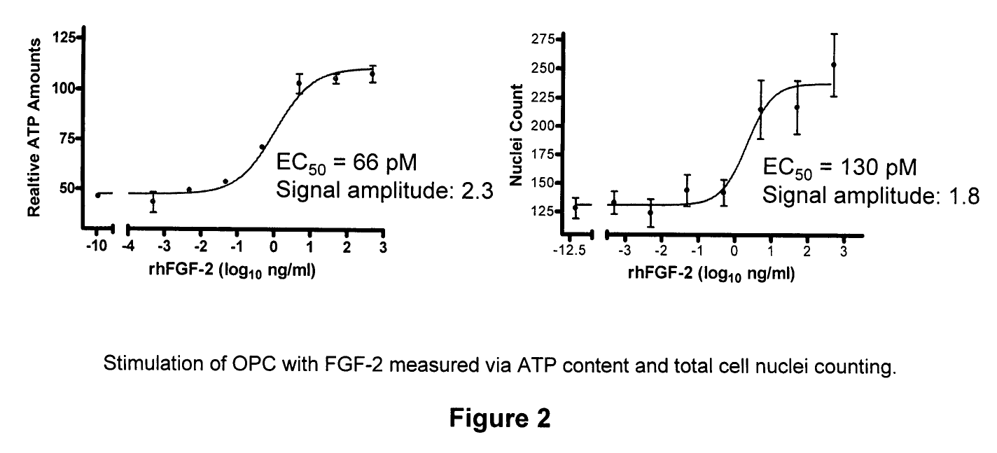

In this experiment, the goal was to develop a high-throughput assay to identify protein factors that induce the proliferation of OPC. For this assay, primary rat OPC, prepared and maintained as described in Examples 1 and 2 were used. 4,000-5,000 cells were plated per well in a 96 PDL-coated plate (Beckton Dickinson, Franklin Lakes, N.J.) in DMEM (Invitrogen, Carlsbad, Calif.). The cells were incubated for 4 days at 37 C and 5% CO2 in the presence or absence of rFGF-2 (R & D Systems, Minneapolis, Minn.) before measuring the ATP amounts with cell titer glo reagent (Promega, Madison, Wis.). For the ATP detection the cell supernatant was removed and 100 μl of a 50% cell titer glo solution diluted with PBS (Mediatech, Herndon, Va.) was added to lyse the cells. After approximately 15 to 30 min of incubation at room temperature luminescence was recorded as a relative measure for ATP content in the cells. An increase in ATP signal is indicative of an increase in cell numbers (FIG. 2). In this experiment, a comparison of the effects of recombinant FGF-2, a known proliferation stimulator of OPC, (Bogler et al., Proc. Natl. Acad. Sci., (USA) 16:6368-6372 (1990); McKinnon et al., Neuron, 5(5):603-614 (1990), between the cell titer glo assay and the total cell nuclei count is shown. Both measurements showed similar results with respect to the EC50 dose and signal amplitude. However, a second factor that might contribute to the ATP signal is the actual size of the cells. OPC undergo a vast morphological change during differentiation (Armstrong, Methods, 16(3):282-292 (1998)). In particular, the formation of cell extensions and an increase in cell body size could result in an increase in ATP amounts even at constant cell numbers. In the screen no discrimination between the contribution of proliferation and differentiation to the ATP signal was attempted.

### Example 5

### High Throughput Screening of Five Prime Therapeutics' Protein Library

The high throughput OPC assay described in Example 4 was used to screen a protein library. This library contained approximately 4000 human secreted proteins and soluble receptors, present in the supernatant of 293T cells expressing the protein in 48 poly-d-lysine coated 96-well plates (Beckton Dickinson, Franklin Lakes, N.J.). Commercially available human recombinant proteins FGF-2, IGF-1 or BMP-4 (R & D Systems, Minneapolis, Minn.) were used as external controls. Internally produced FGF-2 and IGF-1 served as internal controls for the quality of the library expression. Each member of the library was assayed once for activity.

Approximately 12 protein plates were screened per week during a 6 week period. FIG. 3 illustrates an example of such a screening result. Each well-read is represented by a bar in the diagram. The first 8 bars represent the readings from the first column of the plate, followed by the second column until column 12 of the 96 well assay plate. The positive internal and external controls are marked. FIG. 3 shows one protein (CLN00528015) that promoted an increase in ATP levels of the OPC culture. The high throughput screening data was analyzed by calculating the standard deviation (sigma) from median for each test protein using the following formula:

Standard deviation from median(sigma)=[RLU(well)−[RLU(blank)]/sigma(blank).

where “RLU (well)” represented the measured luminescence from a particular test compound well and “RLU (blank)” was calculated the following way. The median and the standard deviation of the raw value of the wells containing test compounds were calculated. For example, in the typical 80-compound plate layout, qualified wells were ranged from Column 2 to Column 11, which contained the expressed library proteins while non-qualified wells were in column 1 and 12, containing the external controls. Then, test compound wells with RLU values that were outside of median by plus or minus 2 standard deviations (sigma) were rejected. The remaining test compound wells were then used to recalculate a new median and a new standard deviation. The RLU (blank) was defined as the new, recalculated median value and the sigma (blank) was defined as the new, recalculated standard deviation.

Test proteins that yielded a measurement at least 2 sigma above or below the median calculated in accordance to the formula were designated as hits in the OPC assay. Table 1, shown below, provides a list of hits from the assay. Information included in Table 1 includes an internal Clone ID number, the representative annotation from public databases such as PubMed (NIH, Bethesda, Md.) and the sigma from median value from the assay.

TABLE 1

| Clone ID | Cluster Annotation | Sigma from mean |
|---|---|---|
| CLN00859148 | phospholipase A2, group V | 24.87 |
| CLN00865330 | chromosome 17 open reading frame 27 | 15.57 |
| CLN00853320 | PRO1097 [Homo sapiens] | 12.77 |
| CLN00876990 | none | 10.31 |
| CLN00942081 | neuregulin 3 | 9.96 |
| CLN00868989 | hypothetical protein MGC39606 | 9.12 |
| CLN00554611 | hypothetical protein FLJ12121 [Homo sapiens] | 6.67 |
| CLN00940208 | transmembrane protease, serine 3 | 6.33 |
| CLN00932074 | fibronectin leucine rich transmembrane protein 3 | 5.94 |
| CLN00736434 | none | 5.09 |
| CLN00938770 | chromosome 10 open reading frame 61 | 4.85 |
| CLN00877127 | none | 4.46 |
| CLN00932227 | hypothetical protein XP_211075 [Homo sapiens] | 4.37 |
| CLN00938850 | KIAA0527 protein | 4.13 |
| CLN00870230 | Fas apoptotic inhibitory molecule 3 | 4.09 |
| CLN00938585 | MEGF10 protein | 4.05 |
| CLN00860842 | hypothetical protein XP_097184 [Homo sapiens] | 3.99 |
| CLN00861035 | unnamed protein product [Homo sapiens] | 3.86 |
| CLN00860791 | unnamed protein product [Homo sapiens] | 3.69 |
| CLN00870451 | jumping translocation breakpoint | 3.69 |
| CLN00865318 | mesoderm development candidate 2 | 3.58 |
| CLN00938858 | protein tyrosine phosphatase, receptor type, U | 3.45 |
| CLN00542945 | hypothetical protein MGC34647 | 3.39 |
| CLN00871450 | tumor necrosis factor (ligand) superfamily, member 14 | 3.18 |
| CLN00913183 | hypothetical protein FLJ20232 | 3.17 |
| CLN00876790 | hypothetical protein XP_211757 [Homo sapiens] | 3.13 |
| CLN00923155 | hypothetical protein XP_098754 [Homo sapiens] | 3.11 |
| CLN00582243 | hypothetical gene LOC150207 chromosome 19 open reading frame 27 similar | 3.07 |
| | to C19orf27 protein | |
| CLN00878644 | FXYD domain containing ion transport regulator 1 (phospholemman) | 3 |
| CLN00933908 | OTTHUMP00000039698 | 2.93 |
| CLN00872016 | lymphocyte transmembrane adaptor 1 | 2.93 |
| CLN00860886 | unnamed protein product [Homo sapiens] | 2.84 |
| CLN00938643 | chromosome 21 open reading frame 51 | 2.81 |
| CLN00733775 | complement component 1, q subcomponent, gamma polypeptide | 2.77 |
| CLN00878190 | tumor necrosis factor receptor superfamily, member 12A | 2.76 |
| CLN00877039 | phosphatidylinositol glycan, class S | 2.71 |
| CLN00823103 | chromosome 1 open reading frame 56 | 2.66 |
| CLN00874475 | interleukin 12 receptor, beta 1 | 2.65 |
| CLN00625748 | tumor necrosis factor (ligand) superfamily, member 4 (tax-transcriptionally | 2.64 |
| | activated glycoprotein 1, 34 kDa) | |
| CLN00850182 | none | 2.6 |
| CLN00929996 | unnamed protein product [Homo sapiens] | 2.6 |
| CLN00878175 | a disintegrin and metalloproteinase domain 22 | 2.57 |
| CLN00795173 | Fas (TNFRSF6)-associated via death domain | 2.57 |
| CLN00860841 | hypothetical protein LOC284033 | 2.56 |
| CLN00875342 | transmembrane protein vezatin | 2.44 |
| CLN00870168 | tumor necrosis factor receptor superfamily, member 1A | 2.44 |
| CLN00849377 | none | 2.42 |
| CLN00874596 | unc-5 homolog C (C. elegans) | 2.42 |
| CLN00942038 | hypothetical LOC387649 | 2.41 |
| CLN00870314 | Xg blood group (pseudoautosomal boundary-divided on the X chromosome) | 2.41 |
| CLN00625649 | stromal cell derived factor receptor 1 | 2.39 |
| CLN00874079 | prepronociceptin | 2.38 |
| CLN00770902 | similar to hephaestin; haphaestin; sex linked anemia [Homo sapiens] | 2.37 |
| CLN00733878 | frizzled-related protein | 2.35 |
| CLN00872067 | hypothetical protein FLJ10579 | 2.33 |
| CLN00921368 | hypothetical protein XP_212039 [Homo sapiens] | 2.31 |
| CLN00878823 | bone morphogenetic protein receptor, type II (serine/threonine kinase) | 2.3 |
| CLN00875352 | similar to hypothetical protein FLJ38101 [Homo sapiens] similar to CG8009-PA | 2.28 |
| | [Homo sapiens] | |
| CLN00736352 | none | 2.27 |
| CLN00773284 | interleukin 1, alpha | 2.27 |
| CLN00874304 | neuromedin B | 2.26 |
| CLN00933988 | chromosome 9 open reading frame 47 | 2.24 |
| CLN00933977 | hypothetical protein XP_108278 [Homo sapiens] | 2.23 |
| CLN00869121 | hypothetical protein MGC4368 | 2.22 |
| CLN00874765 | none | 2.21 |
| CLN00929968 | GWSI6489 [Homo sapiens] | 2.21 |
| CLN00875524 | sarcoglycan zeta | 2.21 |
| CLN00878231 | F11 receptor | 2.2 |
| CLN00849356 | none | 2.19 |
| CLN00528436 | steroid sensitive gene 1 | 2.19 |
| CLN00658098 | hypothetical protein MGC10946 | 2.15 |
| CLN00874814 | colony stimulating factor 1 (macrophage) | 2.14 |
| CLN00546831 | Epstein-Barr virus induced gene 3 | 2.13 |
| CLN00871329 | tumor necrosis factor (TNF superfamily, member 2) topoisomerase (DNA) III | 2.11 |
| | beta, 2 | |
| CLN00658120 | ORM1-like 3 (S. cerevisiae) | 2.08 |
| CLN00921353 | unnamed protein product [Homo sapiens] | 2.07 |
| CLN00921805 | unnamed protein product [Homo sapiens] | 2.05 |
| CLN00869032 | hypothetical protein [Homo sapiens] | 2.04 |
| CLN00897720 | toll-like receptor 5 | 2.01 |
| CLN00916449 | hypothetical protein LOC130576 | 2.01 |

TABLE 2

| | | | | | | | Sigma |
|---|---|---|---|---|---|---|---|
| Assayed | Parent | | | Cluster Pfam | Gene | | from |
| Clone ID | Protein ID | Construct | Classification | Domains | Name | Cluster Annotation | Mean |
| CLN00859148 | CLN00173156 | CLN00173156 | SECRETED | Phospholip_{—} | PLA2G5 | phospholipase A2, | 24.87 |
| | | | | A2_1 | | group V | |
| CLN00865330 | CLN00455998 | CLN00455998 | UB_ligase | zf-C3HC4 | C17orf27 | chromosome 17 open | 15.57 |
| | | | | | | reading frame 27 | |
| CLN00853320 | 7770125 | 7770125 | SECRETED | no_pfam | NA | PRO1097 | 12.77 |
| | | | | | | [Homo sapiens] | |
| CLN00876990 | 314_aa.genscan | 314_aa.genscan | none | none | NA | none | 10.31 |
| CLN00942081 | 55665166 | 55665166_1-360 | STM | EGF | NRG3 | neuregulin 3 | 9.96 |
| | | | TypeI_membrane | Neuregulin | | | |
| CLN00868989 | 30353751 | 30353751 | OTHER | no_pfam | MGC39606 | hypothetical protein | 9.12 |
| | | | | | | MGC39606 | |
| CLN00554611 | Np_079254 | NP_079254 | SECRETED | no_pfam | NA | hypothetical protein | 6.67 |
| | | | | | | FLJ12121 | |
| | | | | | | [Homo sapiens] | |
| CLN00940208 | NP_076927 | NP_076927_71-454 | STM | Ldl_recept_a | TMPRSS3 | transmembrane | 6.33 |
| | | | TypeII_membrane | Trypsin | | protease, serine 3 | |
| CLN00932074 | 7959199 | 7959199_1-410 | STM | LRRCT | FLRT3 | fibronectin leucine | 5.94 |
| | | | TypeI_membrane | LRRNT | | rich transmembrane | |
| | | | | | | protein 3 | |
| CLN00736434 | CLN00144017.a | CLN00144017.a | none | none | NA | none | 5.09 |
| CLN00938770 | 37183162 | 37183162_1-579 | STM | DUF1619 | C10orf61 | chromosome 10 open | 4.85 |
| | | | TypeI_membrane | | | reading frame 61 | |
| CLN00877127 | 204_aa.genscan | 204_aa.genscan | none | none | NA | none | 4.46 |
| CLN00932227 | 27482390 | 27482390 | SECRETED | no_pfam | LOC283507 | hypothetical protein | 4.37 |
| | | | | | | XP_211075 | |
| | | | | | | [Homo sapiens] | |
| CLN00938850 | 22044017 | 22044017_1-575 | STM | Xlink | KIAA0527 | KIAA0527 protein | 4.13 |
| | | | TypeI_membrane | | | | |
| CLN00870230 | 30584491 | 30584491_1-189 | STM | ig V-set | FAIM3 | Fas apoptotic | 4.09 |
| | | | TypeI_membrane | | | inhibitory | |
| | | | | | | molecule 3 | |
| CLN00938585 | 51476585 | 51476585_1-855 | STM | EGF | MEGF10 | MEGF10 protein | 4.05 |
| | | | TypeI_membrane | Laminin_EGF | | | |
| | | | | EMI EGF_2 | | | |
| CLN00860842 | 27483988 | 27483988 | SECRETED | no_pfam | NA | hypothetical protein | 3.99 |
| | | | | | | XP_097184 | |
| | | | | | | [Homo sapiens] | |
| CLN00861035 | 21756729 | 21756729 | SECRETED | no_pfam | NA | unnamed protein | 3.86 |
| | | | | | | product | |
| | | | | | | [Homo sapiens] | |
| CLN00860791 | 34529527 | 34529527 | SECRETED | no_pfam | NA | unnamed protein | 3.69 |
| | | | | | | product | |
| | | | | | | [Homo sapiens] | |
| CLN00870451 | NP_006685 | NP_006685_1-92 | STM | JTB | JTB | jumping translocation | 3.69 |
| | | | TypeI_membrane | | | breakpoint | |
| CLN00865318 | CLN00042589 | CLN00042589 | SECRETED | no_pfam | MESDC2 | mesoderm development | 3.58 |
| | | | | | | candidate 2 | |
| CLN00938858 | 1890660 | 1890660_1-746 | STM | fn3 | PTPRU | protein tyrosine | 3.45 |
| | | | TypeI_membrane | Y_phosphatase | | phosphatase, | |
| | | | | MAM | | receptor type, U | |
| CLN00542945 | NP_689669 | NP_689669 | SECRETED | no_pfam | MGC34647 | hypothetical protein | 3.39 |
| | | | | | | MGC34647 | |
| CLN00871450 | 13124597 | NP_001842_1-23_{—} | STM | TNF | TNFSF14 | tumor necrosis factor | 3.18 |
| | | ECOR1_13124597_{—} | TypeII_membrane | | | (ligand) superfamily, | |
| | | 78-240 | | | | member 14 | |
| CLN00913183 | 50949569 | NP_001842_1-23_{—} | STM | no_pfam | RP5- | hypothetical protein | 3.17 |
| | | ECOR1_50949569_{—} | TypeII_membrane | | 1104E15.5 | FLJ20232 | |
| | | 47-148 | | | | | |
| CLN00876790 | 27480441 | 27480441_1-68 | STM | no_pfam | NA | hypothetical protein | 3.13 |
| | | | TypeI_membrane | | | XP_211757 | |
| | | | | | | [Homo sapiens] | |
| CLN00923155 | 22046814 | 22046814 | SECRETED | no_pfam | NA | hypothetical protein | 3.11 |
| | | | | | | XP_098754 | |
| | | | | | | [Homo sapiens] | |
| CLN00582243 | 12652811 | 12652811 | SECRETED | no_pfam | C19orf27 | hypothetical gene | 3.07 |
| | | | | | LOC150207 | LOC150207 | |
| | | | | | | chromosome 19 open | |
| | | | | | | reading frame 27 | |
| | | | | | | similar to C19orf27 | |
| | | | | | | protein | |
| CLN00878644 | NP_068702 | NP_068702_1-35 | STM | ATP1G1_{—} | FXYD1 | FXYD domain | 3 |
| | | | TypeI_membrane | PLM_{—} | | containing ion | |
| | | | | MAT8 | | transport | |
| | | | | | | regulator 1 | |
| | | | | | | (phospholemman) | |
| CLN00872016 | 25989486 | NP_001842_1-23_{—} | STM | no_pfam | LAX1 | lymphocyte | 2.93 |
| | | ECOR1_25989486_{—} | TypeII_membrane | | | transmembrane | |
| | | 69-398 | | | | adaptor 1 | |
| CLN00933908 | 29742396 | 29742396 | SECRETED | Colipase_C | LOC340204 | OTTHUMP00000039698 | 2.93 |
| CLN00860886 | 34527995 | 34527995 | SECRETED | no_pfam | NA | unnamed protein | 2.84 |
| | | | | | | product | |
| | | | | | | [Homo sapiens] | |
| CLN00938643 | NP_478062 | NP_478062 | SECRETED | no_pfam | C21orf51 | chromosome 21 open | 2.81 |
| | | | | | | reading frame 51 | |
| CLN00733775 | CLN00232956 | CLN00232956 | SECRETED | C1q Collagen | C1QG | complement component | 2.77 |
| | | | | TNF | | 1, q subcomponent, | |
| | | | | | | gamma polypeptide | |
| CLN00878190 | NP_057723 | NP_057723_1-80 | STM | no_pfam | TNFRSF12A | tumor necrosis factor | 2.76 |
| | | | TypeI_membrane | | | receptor superfamily, | |
| | | | | | | member 12A | |
| CLN00877039 | 16550253 | 16550253_1-105 | STM | no_pfam | PIGS | phosphatidylinositol | 2.71 |
| | | | TypeI_membrane | | | glycan, class S | |
| CLN00823103 | NP_060330 | NP_060330 | SECRETED | no_pfam | C1orf56 | chromosome 1 open | 2.66 |
| | | | | | | reading frame 56 | |
| CLN00874475 | NP_005526 | NP_005526_1-495 | STM | fn3 | IL12RB1 | interleukin 12 | 2.65 |
| | | | TypeI_membrane | | | receptor, beta 1 | |
| CLN00625748 | CLN00540586 | CLN00540586 | STM | TNF | TNFSF4 | tumor necrosis factor | 2.64 |
| | | | TypeII_membrane | | | (ligand) superfamily, | |
| | | | | | | member 4 (tax- | |
| | | | | | | transcriptionally | |
| | | | | | | activated glycoprotein | |
| | | | | | | 1, 34 kDa) | |
| CLN00850182 | CLN00223180.a | CLN00223180.a | none | none | NA | none | 2.6 |
| CLN00929996 | 34531092 | 34531092 | SECRETED | no_pfam | NA | unnamed protein | 2.6 |
| | | | | | | product | |
| | | | | | | [Homo sapiens] | |
| CLN00878175 | NP_068369 | NP_068369_1-598 | STM | Reprolysin | ADAM22 | a disintegrin and | 2.57 |
| | | | TypeI_membrane | Pep_M12B_{—} | | metalloproteinase | |
| | | | | propep EGF_2 | | domain 22 | |
| | | | | Disintegrin | | | |
| CLN00795173 | NP_003815 | NP_003815 | OTHER | Death DED | FADD | Fas (TNFRSF6)- | 2.57 |
| | | | | | | associated via | |
| | | | | | | death domain | |
| CLN00860841 | 27482797 | 27482797 | SECRETED | no_pfam | LOC284033 | hypothetical protein | 2.56 |
| | | | | | | LOC284033 | |
| CLN00870168 | NP_001056 | NP_001056_1-205 | STM | TNFR_c6 | TNFRSF1A | tumor necrosis factor | 2.44 |
| | | | TypeI_membrane | Death | | receptor superfamily, | |
| | | | | | | member 1A | |
| CLN00875342 | 11596170 | NP_001842_1-23_{—} | STM | no_pfam | VEZATIN | transmembrane protein | 2.44 |
| | | ECOR1_11596170_{—} | TypeI_membrane | | | vezatin | |
| | | 71-460 | | | | | |
| CLN00874596 | NP_003719 | NP_003719_1-284 | STM | TSP_1 I-set | UNC5C | unc-5 homolog C | 2.42 |
| | | | TypeI_membrane | Death ZU5 | | (C. elegans) | |
| CLN00849377 | CLN00516788.a | CLN00516788.a | none | none | NA | none | 2.42 |
| CLN00942038 | 51468555 | 51468555 | SECRETED | no_pfam | LOC387649 | hypothetical LOC387649 | 2.41 |
| CLN00870314 | NP_780778 | NP_780778_1-130 | STM | no_pfam | XG | Xg blood group | 2.41 |
| | | | TypeI_membrane | | | (pseudoautosomal | |
| | | | | | | boundary-divided | |
| | | | | | | on the X chromosome) | |
| CLN00625649 | NP_059429 | NP_059429_1-221 | STM | I-set ig | SDFR1 | stromal cell derived | 2.39 |
| | | | TypeI_membrane | V-set | | factor receptor 1 | |
| CLN00874079 | NP_006219 | NP_001842_1-23_{—} | SECRETED | Opiods_{—} | PNOC | prepronociceptin | 2.38 |
| | | ECOR1_NP_{—} | | neuropep | | | |
| | | 006219_97-126 | | | | | |
| CLN00770902 | 37541459 | 37541459 | SECRETED | Cu-oxidase_3 | NA | similar to hephaestin; | 2.37 |
| | | | | | | haphaestin; sex linked | |
| | | | | | | anemia [Homo sapiens] | |
| CLN00733878 | CLN00271203 | CLN00271203 | SECRETED | Fz NTR | FRZB | frizzled-related protein | 2.35 |
| CLN00872067 | NP_060615 | NP_001842_1-23_{—} | STM | no_pfam | FAM82C | hypothetical protein | 2.33 |
| | | ECOR1_NP_{—} | TypeII_membrane | | | FLJ10579 | |
| | | 060615_138-470 | | | | | |
| CLN00921368 | 27498551 | 27498551 | SECRETED | no_pfam | NA | hypothetical protein | 2.31 |
| | | | | | | XP_212039 | |
| | | | | | | [Homo sapiens] | |
| CLN00878823 | NP_001195 | NP_001195_1-150 | STM | Pkinase | BMPR2 | bone morphogenetic | 2.3 |
| | | | TypeI_membrane | Activin_recp | | protein receptor, | |
| | | | | | | type II (serine/ | |
| | | | | | | threonine kinase) | |
| CLN00875352 | 17486578 | NP_001842_1-23_{—} | STM | no_pfam | NA | similar to hypothetical | 2.28 |
| | | ECOR1_17486578_{—} | TypeII_membrane | | | protein FLJ38101 | |
| | | 61-129 | | | | [Homo sapiens] | |
| | | | | | | similar to CG8009-PA | |
| | | | | | | [Homo sapiens] | |
| CLN00736352 | CLN00202085.a | CLN00202085.a | none | none | NA | none | 2.27 |
| CLN00773284 | CLN00489263 | NP_001842_1-23_{—} | SECRETED | IL1 | IL1A | interleukin 1, alpha | 2.27 |
| | | ECOR1_{—} | | IL1_propep | | | |
| | | CLN00489263_{—} | | | | | |
| | | 113-206 | | | | | |
| CLN00874304 | 20141490 | NP_001842_1-23_{—} | SECRETED | Bombesin | NMB | neuromedin B | 2.26 |
| | | ECOR1_20141490_{—} | | | | | |
| | | 24-55 | | | | | |
| CLN00933988 | 21753982 | 21753982 | SECRETED | no_pfam | C9orf47 | chromosome 9 open | 2.24 |
| | | | | | | reading frame 47 | |
| CLN00933977 | 18569328 | 18569328 | SECRETED | no_pfam | NA | hypothetical protein | 2.23 |
| | | | | | | XP_108278 | |
| | | | | | | [Homo sapiens] | |
| CLN00869121 | 13097804 | 13097804 | STM | no_pfam | C17orf62 | hypothetical protein | 2.22 |
| | | | TypeII_membrane | | | MGC4368 | |
| CLN00875524 | NP_631906 | NP_001842_1-23_{—} | STM | Sarcoglycan_1 | SGCZ | sarcoglycan zeta | 2.21 |
| | | ECOR1_NP_{—} | TypeII_membrane | | | | |
| | | 631906_187-299 | | | | | |
| CLN00874765 | 298_aa.genscan | 298_aa.genscan | none | none | NA | none | 2.21 |
| CLN00929968 | 37181600 | 37181600 | SECRETED | no_pfam | NA | GWSI6489 [Homo sapiens] | 2.21 |
| CLN00878231 | NP_653087 | NP_653087_1-238 | STM | I-set ig V-set | F11R | F11 receptor | 2.2 |
| | | | TypeI_membrane | Rhodanese | | | |
| CLN00849356 | CLN00139795.a | CLN00139795.a | none | none | NA | none | 2.19 |
| CLN00528436 | 22044951 | 22044951 | SECRETED | no_pfam | URB | steroid sensitive | 2.19 |
| | | | | | | gene 1 | |
| CLN00658098 | NP_085049 | NP_085049 | SECRETED | no_pfam | MGC10946 | hypothetical protein | 2.15 |
| | | | | | | MGC10946 | |
| CLN00874814 | NP_757351 | NP_757351_1-446 | STM | CSF-1 | CSF1 | colony stimulating | 2.14 |
| | | | | | | factor 1 (macrophage) | |
| CLN00546831 | NP_005746 | NP_005746 | SECRETED | fn3 | EBI3 | Epstein-Barr virus | 2.13 |
| | | | | | | induced gene 3 | |
| CLN00871329 | NP_000585 | NP_001842_1-23_{—} | STM | TNF | TNF | tumor necrosis factor | 2.11 |
| | | ECOR1_NP_{—} | TypeII_membrane | | | (TNF superfamily, | |
| | | 000585_75-233 | | | | member 2) topoisomerase | |
| | | | | | | (DNA) III beta, 2 | |
| CLN00658120 | 7106808 | 7106808 | SECRETED | no_pfam | ORMDL3 | ORM1-like 3 | 2.08 |
| | | | | | | (S. cerevisiae) | |
| CLN00921353 | 34533908 | 34533908 | SECRETED | no_pfam | NA | unnamed protein | 2.07 |
| | | | | | | product | |
| | | | | | | [Homo sapiens] | |
| CLN00921805 | 34535680 | 34535680 | SECRETED | no_pfam | NA | unnamed protein | 2.05 |
| | | | | | | product | |
| | | | | | | [Homo sapiens] | |
| CLN00869032 | 31873423 | 31873423 | SECRETED | no_pfam | LOC654433 | hypothetical protein | 2.04 |
| | | | | | | [Homo sapiens] | |
| CLN00897720 | 20140433 | 20140433_1-639 | STM | TIR LRR_1 | TLR5 | toll-like receptor 5 | 2.01 |
| | | | TypeI_membrane | LRRCT | | | |
| CLN00916449 | 20536316 | 20536316 | SECRETED | no_pfam | LOC130576 | hypothetical protein | 2.01 |
| | | | | | | LOC130576 | |

TABLE 3

| FP ID | P1 SEQ. ID | N1 SEQ. ID | Protein/Construct |
|---|---|---|---|
| HG1023454 | SEQ. ID. NO. 1 | SEQ. ID. NO. 114 | NP_001842_1-23_ECOR1_NP_055173_44-219 |
| HG1023455 | SEQ. ID. NO. 2 | SEQ. ID. NO. 115 | 27482390 |
| HG1023456 | SEQ. ID. NO. 3 | SEQ. ID. NO. 116 | 12652811 |
| HG1023457 | SEQ. ID. NO. 4 | SEQ. ID. NO. 117 | 34535680 |
| HG1023458 | SEQ. ID. NO. 5 | SEQ. ID. NO. 118 | NP_001842_1-23_ECOR1_NP_003800_91-249 |
| HG1023459 | SEQ. ID. NO. 6 | SEQ. ID. NO. 119 | 30353751 |
| HG1023460 | SEQ. ID. NO. 7 | SEQ. ID. NO. 120 | 21750618 |
| HG1023461 | SEQ. ID. NO. 8 | SEQ. ID. NO. 121 | 40288201_1-218 |
| HG1023462 | SEQ. ID. NO. 9 | SEQ. ID. NO. 122 | CLN00202085.a |
| HG1023463 | SEQ. ID. NO. 10 | SEQ. ID. NO. 123 | NP_068369_1-598 |
| HG1023464 | SEQ. ID. NO. 11 | SEQ. ID. NO. 124 | NP_001195_1-150 |
| HG1023465 | SEQ. ID. NO. 12 | SEQ. ID. NO. 125 | 27480441_1-68 |
| HG1023466 | SEQ. ID. NO. 13 | SEQ. ID. NO. 126 | NP_001786_1-593 |
| HG1023467 | SEQ. ID. NO. 14 | SEQ. ID. NO. 127 | NP_001842_1-23_ECOR1_13477273_104-886 |
| HG1023468 | SEQ. ID. NO. 15 | SEQ. ID. NO. 128 | NP_001842_1-23_ECOR1_11596170_71-460 |
| HG1023469 | SEQ. ID. NO. 16 | SEQ. ID. NO. 129 | 18569328 |
| HG1023470 | SEQ. ID. NO. 17 | SEQ. ID. NO. 130 | NP_005526_1-495 |
| HG1023471 | SEQ. ID. NO. 18 | SEQ. ID. NO. 131 | 39645305_1-526_17939658_233-464_C237S |
| HG1023472 | SEQ. ID. NO. 19 | SEQ. ID. NO. 132 | NP_059429_1-221 |
| HG1023473 | SEQ. ID. NO. 20 | SEQ. ID. NO. 133 | NP_001842_1-23_ECOR1_6832905_18-130 |
| HG1023474 | SEQ. ID. NO. 21 | SEQ. ID. NO. 134 | CLN00131297 |
| HG1023475 | SEQ. ID. NO. 22 | SEQ. ID. NO. 135 | CLN00144017.a |
| HG1023476 | SEQ. ID. NO. 23 | SEQ. ID. NO. 136 | 37183162_1-579 |
| HG1023477 | SEQ. ID. NO. 24 | SEQ. ID. NO. 137 | 298_aa.genscan |
| HG1023478 | SEQ. ID. NO. 25 | SEQ. ID. NO. 138 | 37541459 |
| HG1023479 | SEQ. ID. NO. 26 | SEQ. ID. NO. 139 | 34529527 |
| HG1023480 | SEQ. ID. NO. 27 | SEQ. ID. NO. 140 | NP_001842_1-23_ECOR1_NP_001770_28-233 |
| HG1023481 | SEQ. ID. NO. 28 | SEQ. ID. NO. 141 | NP_001842_1-23_ECOR1_17486578_61-129 |
| HG1023482 | SEQ. ID. NO. 29 | SEQ. ID. NO. 142 | 55665166_1-360 |
| HG1023483 | SEQ. ID. NO. 30 | SEQ. ID. NO. 143 | NP_001842_1-23_ECOR1_25989486_69-398 |
| HG1023484 | SEQ. ID. NO. 31 | SEQ. ID. NO. 144 | 21753982 |
| HG1023485 | SEQ. ID. NO. 32 | SEQ. ID. NO. 145 | 22044017_1-575 |
| HG1023486 | SEQ. ID. NO. 33 | SEQ. ID. NO. 146 | NP_057723_1-80 |
| HG1023487 | SEQ. ID. NO. 34 | SEQ. ID. NO. 147 | proteinkinase270A_1-403 |
| HG1023488 | SEQ. ID. NO. 35 | SEQ. ID. NO. 148 | NP_055695_1-170 |
| HG1023489 | SEQ. ID. NO. 36 | SEQ. ID. NO. 149 | 1890660_1-746 |
| HG1023490 | SEQ. ID. NO. 37 | SEQ. ID. NO. 150 | 16550253_1-105 |
| HG1023491 | SEQ. ID. NO. 38 | SEQ. ID. NO. 151 | NP_478062 |
| HG1023492 | SEQ. ID. NO. 39 | SEQ. ID. NO. 152 | 22047619 |
| HG1023493 | SEQ. ID. NO. 40 | SEQ. ID. NO. 153 | NP_001842_1-23_ECOR1_NP_612445_37-100 |
| HG1023494 | SEQ. ID. NO. 41 | SEQ. ID. NO. 154 | CLN00493523 |
| HG1023495 | SEQ. ID. NO. 42 | SEQ. ID. NO. 155 | 20140433_1-639 |
| HG1023496 | SEQ. ID. NO. 43 | SEQ. ID. NO. 156 | CLN00042589 |
| HG1023497 | SEQ. ID. NO. 44 | SEQ. ID. NO. 157 | NP_076927_71-454 |
| HG1023498 | SEQ. ID. NO. 45 | SEQ. ID. NO. 158 | 34527995 |
| HG1023499 | SEQ. ID. NO. 46 | SEQ. ID. NO. 159 | 21756729 |
| HG1023500 | SEQ. ID. NO. 47 | SEQ. ID. NO. 160 | 22046814 |
| HG1023501 | SEQ. ID. NO. 48 | SEQ. ID. NO. 161 | CLN00223180.a |
| HG1023502 | SEQ. ID. NO. 49 | SEQ. ID. NO. 162 | CLN00139795.a |
| HG1023503 | SEQ. ID. NO. 50 | SEQ. ID. NO. 163 | CLN00129208 |
| HG1023504 | SEQ. ID. NO. 51 | SEQ. ID. NO. 164 | CLN00540586 |
| HG1023505 | SEQ. ID. NO. 52 | SEQ. ID. NO. 165 | 51476585_1-855 |
| HG1023506 | SEQ. ID. NO. 53 | SEQ. ID. NO. 166 | CLN00232956 |
| HG1023507 | SEQ. ID. NO. 54 | SEQ. ID. NO. 167 | NP_001842_1-23_ECOR1_13124597_78-240 |
| HG1023508 | SEQ. ID. NO. 55 | SEQ. ID. NO. 168 | NP_001842_1-23_ECOR1_NP_055580_29-145 |
| HG1023509 | SEQ. ID. NO. 56 | SEQ. ID. NO. 169 | 20536316 |
| HG1023510 | SEQ. ID. NO. 57 | SEQ. ID. NO. 170 | NP_068702_1-35 |
| HG1023511 | SEQ. ID. NO. 58 | SEQ. ID. NO. 171 | NP_085049 |
| HG1023512 | SEQ. ID. NO. 59 | SEQ. ID. NO. 172 | 2587058_1-413 |
| HG1023513 | SEQ. ID. NO. 60 | SEQ. ID. NO. 173 | 31873423 |
| HG1023514 | SEQ. ID. NO. 61 | SEQ. ID. NO. 174 | NP_001842_1-23_ECOR1_NP_006219_97-126 |
| HG1023515 | SEQ. ID. NO. 62 | SEQ. ID. NO. 175 | NP_001842_1-23_ECOR1_50949569_47-148 |
| HG1023516 | SEQ. ID. NO. 63 | SEQ. ID. NO. 176 | 204_aa.genscan |
| HG1023517 | SEQ. ID. NO. 64 | SEQ. ID. NO. 177 | NP_001842_1-23_ECOR1_NP_631906_187-299 |
| HG1023518 | SEQ. ID. NO. 65 | SEQ. ID. NO. 178 | 51468555 |
| HG1023519 | SEQ. ID. NO. 66 | SEQ. ID. NO. 179 | CLN00161892 |
| HG1023520 | SEQ. ID. NO. 67 | SEQ. ID. NO. 180 | NP_001648_1-183 |
| HG1023521 | SEQ. ID. NO. 68 | SEQ. ID. NO. 181 | 34365357 |
| HG1023522 | SEQ. ID. NO. 69 | SEQ. ID. NO. 182 | NP_060159 |
| HG1023523 | SEQ. ID. NO. 70 | SEQ. ID. NO. 183 | NP_001057_1-257 |
| HG1023524 | SEQ. ID. NO. 71 | SEQ. ID. NO. 184 | CLN00455998 |
| HG1023525 | SEQ. ID. NO. 72 | SEQ. ID. NO. 185 | NP_037363_1-457 |
| HG1023526 | SEQ. ID. NO. 73 | SEQ. ID. NO. 186 | 34531092 |
| HG1023527 | SEQ. ID. NO. 74 | SEQ. ID. NO. 187 | 34533908 |
| HG1023528 | SEQ. ID. NO. 75 | SEQ. ID. NO. 188 | 10435604_1-385 |
| HG1023529 | SEQ. ID. NO. 76 | SEQ. ID. NO. 189 | 30584491_1-189 |
| HG1023530 | SEQ. ID. NO. 77 | SEQ. ID. NO. 190 | NP_689669 |
| HG1023531 | SEQ. ID. NO. 78 | SEQ. ID. NO. 191 | CLN00519958 |
| HG1023532 | SEQ. ID. NO. 79 | SEQ. ID. NO. 192 | CLN00516788.a |
| HG1023533 | SEQ. ID. NO. 80 | SEQ. ID. NO. 193 | NP_001056_1-205 |
| HG1023534 | SEQ. ID. NO. 81 | SEQ. ID. NO. 194 | NP_006685_1-92 |
| HG1023535 | SEQ. ID. NO. 82 | SEQ. ID. NO. 195 | NP_003719_1-284 |
| HG1023536 | SEQ. ID. NO. 83 | SEQ. ID. NO. 196 | NP_001842_1-23_ECOR1_NP_000585_75-233 |
| HG1023537 | SEQ. ID. NO. 84 | SEQ. ID. NO. 197 | NP_001842_1-23_ECOR1_NP_001004419.1_57-194 |
| HG1023538 | SEQ. ID. NO. 85 | SEQ. ID. NO. 198 | 902797_1-320 |
| HG1023539 | SEQ. ID. NO. 86 | SEQ. ID. NO. 199 | NP_001842_1-23_ECOR1_20141490_24-55 |
| HG1023540 | SEQ. ID. NO. 87 | SEQ. ID. NO. 200 | NP_001842_1-23_ECOR1_CLN00489263_113-206 |
| HG1023541 | SEQ. ID. NO. 88 | SEQ. ID. NO. 201 | 314_aa.genscan |
| HG1023542 | SEQ. ID. NO. 89 | SEQ. ID. NO. 202 | 13097804 |
| HG1023543 | SEQ. ID. NO. 90 | SEQ. ID. NO. 203 | NP_079254 |
| HG1023544 | SEQ. ID. NO. 91 | SEQ. ID. NO. 204 | 7959199_1-410 |
| HG1023545 | SEQ. ID. NO. 92 | SEQ. ID. NO. 205 | NP_653087_1-238 |
| HG1023546 | SEQ. ID. NO. 93 | SEQ. ID. NO. 206 | 27498551 |
| HG1023547 | SEQ. ID. NO. 94 | SEQ. ID. NO. 207 | NP_757351_1-446 |
| HG1023548 | SEQ. ID. NO. 95 | SEQ. ID. NO. 208 | CLN00173156 |
| HG1023549 | SEQ. ID. NO. 96 | SEQ. ID. NO. 209 | NP_780778_1-130 |
| HG1023550 | SEQ. ID. NO. 97 | SEQ. ID. NO. 210 | NP_060330 |
| HG1023551 | SEQ. ID. NO. 98 | SEQ. ID. NO. 211 | NP_001842_1-23_ECOR1_NP_060615_138-470 |
| HG1023552 | SEQ. ID. NO. 99 | SEQ. ID. NO. 212 | 27482797 |
| HG1023553 | SEQ. ID. NO. 100 | SEQ. ID. NO. 213 | 27483988 |
| HG1023554 | SEQ. ID. NO. 101 | SEQ. ID. NO. 214 | NP_002498_1-249 |
| HG1023555 | SEQ. ID. NO. 102 | SEQ. ID. NO. 215 | NP_001842_1-23_ECOR1_NP_006841_49-206 |
| HG1023556 | SEQ. ID. NO. 103 | SEQ. ID. NO. 216 | CLN00271203 |
| HG1023557 | SEQ. ID. NO. 104 | SEQ. ID. NO. 217 | 13279053 |
| HG1023558 | SEQ. ID. NO. 105 | SEQ. ID. NO. 218 | 7106808 |
| HG1023559 | SEQ. ID. NO. 106 | SEQ. ID. NO. 219 | NP_005746 |
| HG1023560 | SEQ. ID. NO. 107 | SEQ. ID. NO. 220 | 29742396 |
| HG1023561 | SEQ. ID. NO. 108 | SEQ. ID. NO. 221 | 37181600 |
| HG1023562 | SEQ. ID. NO. 109 | SEQ. ID. NO. 222 | 19338684 |
| HG1023563 | SEQ. ID. NO. 110 | SEQ. ID. NO. 223 | NP_003815 |
| HG1023564 | SEQ. ID. NO. 111 | SEQ. ID. NO. 224 | 37183032 |
| HG1023565 | SEQ. ID. NO. 112 | SEQ. ID. NO. 225 | 22044951 |
| HG1023566 | SEQ. ID. NO. 113 | SEQ. ID. NO. 226 | 7770125 |

TABLE 4

| | Assayed | | | Start | End | Protein/Construct |
|---|---|---|---|---|---|---|
| FPT ID | Clone ID | Protein/Construct | Pfam Domain | (aa) | (aa) | Length (aa) |
| HG1023454 | CLN00928633 | NP_001842_1-23_ECOR1_NP_055173_44-219 | Lectin_C | 79 | 189 | 201 |
| HG1023455 | CLN00932227 | 27482390 | no_pfam | | | 124 |
| HG1023456 | CLN00582243 | 12652811 | no_pfam | | | 236 |
| HG1023457 | CLN00921805 | 34535680 | no_pfam | | | 189 |
| HG1023458 | CLN00871125 | NP_001842_1-23_ECOR1_NP_003800_91-249 | TNF | 67 | 184 | 185 |
| HG1023459 | CLN00868989 | 30353751 | no_pfam | | | 78 |
| HG1023460 | CLN00897769 | 21750618 | no_pfam | | | 188 |
| HG1023461 | CLN00919960 | 40288201_1-218 | no_pfam | | | 218 |
| HG1023462 | CLN00736352 | CLN00202085.a | no_pfam | | | 70 |
| HG1023463 | CLN00878175 | NP_068369_1-598 | Pep_M12B_propep | 103 | 180 | 598 |
| HG1023463 | CLN00878175 | NP_068369_1-598 | Disintegrin | 453 | 529 | 598 |
| HG1023463 | CLN00878175 | NP_068369_1-598 | Reprolysin | 239 | 438 | 598 |
| HG1023464 | CLN00878823 | NP_001195_1-150 | Activin_recp | 32 | 131 | 150 |
| HG1023465 | CLN00876790 | 27480441_1-68 | no_pfam | | | 68 |
| HG1023466 | CLN00884956 | NP_001786_1-593 | Cadherin | 483 | 576 | 593 |
| HG1023466 | CLN00884956 | NP_001786_1-593 | Cadherin | 52 | 142 | 593 |
| HG1023466 | CLN00884956 | NP_001786_1-593 | Cadherin | 377 | 470 | 593 |
| HG1023466 | CLN00884956 | NP_001786_1-593 | Cadherin | 156 | 249 | 593 |
| HG1023466 | CLN00884956 | NP_001786_1-593 | Cadherin | 263 | 364 | 593 |
| HG1023467 | CLN00905128 | NP_001842_1-23_ECOR1_13477273_104-886 | PXA | 52 | 175 | 808 |
| HG1023467 | CLN00905128 | NP_001842_1-23_ECOR1_13477273_104-886 | PX | 439 | 548 | 808 |
| HG1023468 | CLN00875342 | NP_001842_1-23_ECOR1_11596170_71-460 | no_pfam | | | 416 |
| HG1023469 | CLN00933977 | 18569328 | no_pfam | | | 162 |
| HG1023470 | CLN00874475 | NP_005526_1-495 | no_pfam | | | 495 |
| HG1023471 | CLN00923348 | 39645305_1-526_17939658_233-464_C237S | ig | 45 | 105 | 763 |
| HG1023471 | CLN00923348 | 39645305_1-526_17939658_233-464_C237S | I-set | 340 | 402 | 763 |
| HG1023471 | CLN00923348 | 39645305_1-526_17939658_233-464_C237S | ig | 231 | 295 | 763 |
| HG1023471 | CLN00923348 | 39645305_1-526_17939658_233-464_C237S | ig | 676 | 743 | 763 |
| HG1023471 | CLN00923348 | 39645305_1-526_17939658_233-464_C237S | C1-set | 560 | 649 | 763 |
| HG1023471 | CLN00923348 | 39645305_1-526_17939658_233-464_C237S | C1-set | 668 | 754 | 763 |
| HG1023472 | CLN00625649 | NP_059429_1-221 | ig | 49 | 104 | 221 |
| HG1023472 | CLN00625649 | NP_059429_1-221 | I-sel | 122 | 218 | 221 |
| HG1023473 | CLN00904832 | NP_001842_1-23_ECOR1_6832905_18-130 | Granin | 102 | 140 | 140 |
| HG1023473 | CLN00904832 | NP_001842_1-23_ECOR1_6832905_18-130 | Granin | 27 | 100 | 140 |
| HG1023474 | CLN00822755 | CLN00131297 | ig | 161 | 210 | 240 |
| HG1023474 | CLN00822755 | CLN00131297 | V-set | 1 | 136 | 240 |
| HG1023475 | CLN00736434 | CLN00144017.a | no_pfam | | | 85 |
| HG1023476 | CLN00938770 | 37183162_1-579 | DUF1619 | 90 | 390 | 579 |
| HG1023477 | CLN00874765 | 298_aa.genscan | no_pfam | | | 131 |
| HG1023478 | CLN00770902 | 37541459 | Cu-oxidase_3 | 99 | 132 | 272 |
| HG1023479 | CLN00860791 | 34529527 | no_pfam | | | 140 |
| HG1023480 | CLN00905066 | NP_001842_1-23_ECOR1_NP_001770_28-233 | no_pfam | | | 232 |
| HG1023481 | CLN00875352 | NP_001842_1-23_ECOR1_17486578_61-129 | no_pfam | | | 95 |
| HG1023482 | CLN00942081 | 55665166_1-360 | EGF | 290 | 328 | 360 |
| HG1023483 | CLN00872016 | NP_001842_1-23_ECOR1_25989486_69-398 | no_pfam | | | 356 |
| HG1023484 | CLN00933988 | 21753982 | no_pfam | | | 183 |
| HG1023485 | CLN00938850 | 22044017_1-575 | Xlink | 38 | 97 | 575 |
| HG1023486 | CLN00878190 | NP_057723_1-80 | no_pfam | | | 80 |
| HG1023487 | CLN00898948 | proteinkinase270A_1-403 | I-set | 62 | 153 | 403 |
| HG1023487 | CLN00898948 | proteinkinase270A_1-403 | Kringle | 316 | 394 | 403 |
| HG1023487 | CLN00898948 | proteinkinase270A_1-403 | Fz | 164 | 301 | 403 |
| HG1023488 | CLN00920019 | NP_055695_1-170 | Lectin_C | 50 | 155 | 170 |
| HG1023489 | CLN00938858 | 1890660_1-746 | fn3 | 286 | 370 | 746 |
| HG1023489 | CLN00938858 | 1890660_1-746 | fn3 | 485 | 578 | 746 |
| HG1023489 | CLN00938858 | 1890660_1-746 | MAM | 27 | 188 | 746 |
| HG1023490 | CLN00877039 | 16550253_1-105 | no_pfam | | | 105 |
| HG1023491 | CLN00938643 | NP_478062 | no_pfam | | | 58 |
| HG1023492 | CLN00849492 | 22047619 | Trypsin | 42 | 177 | 235 |
| HG1023493 | CLN00912948 | NP_001842_1-23_ECOR1_NP_612445_37-100 | no_pfam | | | 89 |
| HG1023494 | CLN00822056 | CLN00493523 | IL6 | 51 | 101 | 189 |
| HG1023494 | CLN00822056 | CLN00493523 | IL6 | 102 | 184 | 189 |
| HG1023495 | CLN00897720 | 20140433_1-639 | LRR_1 | 337 | 360 | 639 |
| HG1023495 | CLN00897720 | 20140433_1-639 | LRR_1 | 503 | 526 | 639 |
| HG1023495 | CLN00897720 | 20140433_1-639 | LRRCT | 607 | 630 | 639 |
| HG1023496 | CLN00865318 | CLN00042589 | no_pfam | | | 138 |
| HG1023497 | CLN00940208 | NP_076927_71-454 | Ldl_recept_a | 1 | 39 | 384 |
| HG1023497 | CLN00940208 | NP_076927_71-454 | Trypsin | 147 | 374 | 384 |
| HG1023498 | CLN00860886 | 34527995 | no_pfam | | | 129 |
| HG1023499 | CLN00861035 | 21756729 | no_pfam | | | 128 |
| HG1023500 | CLN00923155 | 22046814 | no_pfam | | | 104 |
| HG1023501 | CLN00850182 | CLN00223180.a | no_pfam | | | 68 |
| HG1023502 | CLN00849356 | CLN00139795.a | no_pfam | | | 76 |
| HG1023503 | CLN00625745 | CLN00129208 | I-set | 33 | 129 | 193 |
| HG1023504 | CLN00625748 | CLN00540586 | no_pfam | | | 155 |
| HG1023504 | CLN00625748 | CLN00540586 | no_pfam | | | 155 |
| HG1023505 | CLN00938585 | 51476585_1-855 | Laminin_EGF | 152 | 195 | 855 |
| HG1023505 | CLN00938585 | 51476585_1-855 | Laminin_EGF | 634 | 658 | 855 |
| HG1023505 | CLN00938585 | 51476585_1-855 | Laminin_EGF | 368 | 406 | 855 |
| HG1023505 | CLN00938585 | 514765B5_1-855 | EGF | 283 | 307 | 855 |
| HG1023505 | CLN00938585 | 51476585_1-855 | EGF_2 | 499 | 525 | 855 |
| HG1023505 | CLN00938585 | 51476585_1-855 | Laminin_EGF | 759 | 802 | 855 |
| HG1023505 | CLN00938585 | 51476585_1-855 | EGF_2 | 716 | 742 | 855 |
| HG1023505 | CLN00938585 | 51476585_1-855 | EGF_2 | 238 | 264 | 855 |
| HG1023505 | CLN00938585 | 51476585_1-855 | Laminin_EGF | 281 | 317 | 855 |
| HG1023505 | CLN00938585 | 51476585_1-855 | EGF_2 | 195 | 221 | 855 |
| HG1023505 | CLN00938585 | 51476585_1-855 | EGF_2 | 281 | 307 | 855 |
| HG1023505 | CLN00938585 | 51476585_1-855 | EMI | 30 | 102 | 855 |
| HG1023506 | CLN00733775 | CLN00232956 | TNF | 123 | 212 | 215 |
| HG1023506 | CLN00733775 | CLN00232956 | Collagen | 37 | 82 | 215 |
| HG1023506 | CLN00733775 | CLN00232956 | C1q | 91 | 212 | 215 |
| HG1023507 | CLN00871450 | NP_001842_1-23_ECOR1_13124597_78-240 | TNF | 61 | 189 | 189 |
| HG1023508 | CLN00904865 | NP_001842_1-23_ECOR1_NP_055580_29-145 | no_pfam | | | 142 |
| HG1023509 | CLN00916449 | 20536316 | no_pfam | | | 183 |
| HG1023510 | CLN00878644 | NP_068702_1-35 | no_pfam | | | 35 |
| HG1023511 | CLN00658098 | NP_085049 | no_pfam | | | 116 |
| HG1023512 | CLN00914055 | 2587058_1-413 | no_pfam | | | 413 |
| HG1023513 | CLN00869032 | 31873423 | no_pfam | | | 147 |
| HG1023514 | CLN00874079 | NP_001842_1-23_ECOR1_NP_006219_97-126 | no_pfam | | | 57 |
| HG1023515 | CLN00913183 | NP_001842_1-23_ECOR1_50949569_47-148 | no_pfam | | | 127 |
| HG1023516 | CLN00877127 | 204_aa.genscan | no_pfam | | | 118 |
| HG1023517 | CLN00875524 | NP_001842_1-23_ECOR1_NP_631906_187-299 | Sarcoglycan_1 | 28 | 130 | 139 |
| HG1023518 | CLN00942038 | 51468555 | no_pfam | | | 212 |
| HG1023519 | CLN00852080 | CLN00161892 | no_pfam | | | 113 |
| HG1023520 | CLN00923451 | NP_001648_1-183 | EGF | 146 | 181 | 183 |
| HG1023521 | CLN00849297 | 34365357 | no_pfam | | | 219 |
| HG1023522 | CLN00869329 | NP_060159 | no_pfam | | | 141 |
| HG1023523 | CLN00919867 | NP_001057_1-257 | TNFR_c6 | 120 | 161 | 257 |
| HG1023523 | CLN00919867 | NP_001057_1-257 | TNFR_c6 | 40 | 75 | 257 |
| HG1023523 | CLN00919867 | NP_001057_1-257 | TNFR_c6 | 78 | 118 | 257 |
| HG1023524 | CLN00865330 | CLN00455998 | no_pfam | | | 98 |
| HG1023525 | CLN00899057 | NP_037363_1-457 | LRRCT | 336 | 361 | 457 |
| HG1023525 | CLN00899057 | NP_037363_1-457 | LRRNT | 35 | 62 | 457 |
| HG1023526 | CLN00929996 | 34531092 | no_pfam | | | 164 |
| HG1023527 | CLN00921353 | 34533908 | no_pfam | | | 130 |
| HG1023528 | CLN00885080 | 10435604_1-385 | MANEC | 24 | 117 | 385 |
| HG1023529 | CLN00870230 | 30584491_1-189 | V-sel | 16 | 123 | 189 |
| HG1023529 | CLN00870230 | 30584491_1-189 | ig | 30 | 106 | 189 |
| HG1023530 | CLN00542951 | NP_689669 | no_pfam | | | 242 |
| HG1023530 | CLN00542951 | NP_689669 | no_pfam | | | 242 |
| HG1023531 | CLN00800085 | CLN00519958 | no_pfam | | | 99 |
| HG1023532 | CLN00849377 | CLN00516788.a | no_pfam | | | 71 |
| HG1023533 | CLN00870168 | NP_001056_1-205 | TNFR_c6 | 127 | 166 | 205 |
| HG1023533 | CLN00870168 | NP_001056_1-205 | TNFR_c6 | 44 | 81 | 205 |
| HG1023533 | CLN00870168 | NP_001056_1-205 | TNFR_c6 | 84 | 125 | 205 |
| HG1023534 | CLN00870451 | NP_006685_1-92 | JTB | 30 | 92 | 92 |
| HG1023535 | CLN00874596 | NP_003719_1-284 | I-set | 167 | 258 | 284 |
| HG1023536 | CLN00871329 | NP_001842_1-23_ECOR1_NP_000585_75-233 | TNF | 54 | 185 | 185 |
| HG1023537 | CLN00928403 | NP_001842_1-23_ECOR1_NP_001004419.1_57-194 | Lectin_C | 61 | 122 | 163 |
| HG1023538 | CLN00885012 | 902797_1-320 | no_pfam | | | 320 |
| HG1023539 | CLN00874304 | NP_001842_1-23_ECOR1_20141490_24-55 | no_pfam | | | 59 |
| HG1023540 | CLN00773284 | NP_001842_1-23_ECOR1_CLN00489263_113-206 | IL1 | 48 | 119 | 119 |
| HG1023541 | CLN00876990 | 314_aa.genscan | no_pfam | | | 133 |
| HG1023542 | CLN00869121 | 13097804 | no_pfam | | | 187 |
| HG1023543 | CLN00554611 | NP_079254 | no_pfam | | | 191 |
| HG1023544 | CLN00932074 | 7959199_1-410 | LRRCT | 331 | 356 | 410 |
| HG1023544 | CLN00932074 | 7959199_1-410 | LRRNT | 30 | 57 | 410 |
| HG1023545 | CLN00878231 | NP_653087_1-238 | I-set | 38 | 131 | 238 |
| HG1023545 | CLN00878231 | NP_653087_1-238 | ig | 146 | 214 | 238 |
| HG1023545 | CLN00878231 | NP_653087_1-238 | ig | 43 | 111 | 238 |
| HG1023545 | CLN00878231 | NP_653087_1-238 | V-set | 28 | 129 | 238 |
| HG1023546 | CLN00921368 | 27498551 | no_pfam | | | 109 |
| HG1023547 | CLN00874814 | NP_757351_1-446 | CSF-1 | 272 | 446 | 446 |
| HG1023547 | CLN00874814 | NP_757351_1-446 | CSF-1 | 1 | 268 | 446 |
| HG1023548 | CLN00859148 | CLN00173156 | Phospholip_A2_1 | 21 | 61 | 61 |
| HG1023549 | CLN00870314 | NP_780778_1-130 | no_pfam | | | 130 |
| HG1023550 | CLN00823103 | NP_060330 | no_pfam | | | 341 |
| HG1023551 | CLN00872067 | NP_001842_1-23_ECOR1_NP_060615_138-470 | no_pfam | | | 359 |
| HG1023552 | CLN00860841 | 27482797 | no_pfam | | | 128 |
| HG1023553 | CLN00860842 | 27483988 | no_pfam | | | 150 |
| HG1023554 | CLN00927329 | NP_002498_1-249 | TNFR_c6 | 32 | 64 | 249 |
| HG1023554 | CLN00927329 | NP_002498_1-249 | TNFR_c6 | 67 | 107 | 249 |
| HG1023554 | CLN00927329 | NP_002498_1-249 | TNFR_c6 | 109 | 146 | 249 |
| HG1023554 | CLN00927329 | NP_002498_1-249 | TNFR_c6 | 149 | 188 | 249 |
| HG1023555 | CLN00773292 | NP_001842_1-23_ECOR1_NP_006841_49-206 | no_pfam | | | 183 |
| HG1023556 | CLN00733878 | CLN00271203 | NTR | 176 | 226 | 228 |
| HG1023556 | CLN00733878 | CLN00271203 | Fz | 25 | 148 | 228 |
| HG1023557 | CLN00919453 | 13279053 | no_pfam | | | 253 |
| HG1023558 | CLN00658120 | 7106808 | no_pfam | | | 144 |
| HG1023559 | CLN00546831 | NP_005746 | fn3 | 129 | 215 | 229 |
| HG1023560 | CLN00933908 | 29742396 | Colipase_C | 67 | 91 | 121 |
| HG1023561 | CLN00929968 | 37181600 | no_pfam | | | 91 |
| HG1023562 | CLN00603883 | 19338684 | efhand | 103 | 131 | 229 |
| HG1023562 | CLN00603883 | 19338684 | efhand | 187 | 215 | 229 |
| HG1023562 | CLN00603883 | 19338684 | efhand | 139 | 167 | 229 |
| HG1023563 | CLN00795173 | NP_003815 | Death | 98 | 181 | 208 |
| HG1023563 | CLN00795173 | NP_003815 | DED | 4 | 87 | 208 |
| HG1023564 | CLN00851780 | 37183032 | no_pfam | | | 109 |
| HG1023565 | CLN00528436 | 22044951 | no_pfam | | | 950 |
| HG1023566 | CLN00853320 | 7770125 | no_pfam | | | 68 |

TABLE 5

| | Assayed | | | Signal Sequence | Mature Peptide |
|---|---|---|---|---|---|
| FP ID | Clone ID | Protein/Construct | Treevote | Position | Position |
| HG1023454 | CLN00928633 | NP_001842_1-23_ECOR1_NP_055173_44-219 | 0.93 | 1-23 | 24-201 |
| HG1023455 | CLN00932227 | 27482390 | 0.95 | 1-18 | 19-124 |
| HG1023456 | CLN00582243 | 12652811 | 0.97 | 1-20 | 21-236 |
| HG1023457 | CLN00921805 | 34535680 | 0.89 | 1-16 | 17-189 |
| HG1023458 | CLN00871125 | NP_001842_1-23_ECOR1_NP_003800_91-249 | 0.93 | 1-23 | 24-185 |
| HG1023459 | CLN00868989 | 30353751 | 0.49 | 1-31 | 32-78 |
| HG1023460 | CLN00897769 | 21750618 | 0.25 | 1-33 | 34-188 |
| HG1023461 | CLN00919960 | 40288201_1-218 | 0.13 | 1-25 | 26-218 |
| HG1023462 | CLN00736352 | CLN00202085.a | 0.26 | 1-36 | 37-70 |
| HG1023463 | CLN00878175 | NP_068369_1-598 | 1 | 1-25 | 26-598 |
| HG1023464 | CLN00878823 | NP_001195_1-150 | 0.87 | 1-26 | 27-150 |
| HG1023465 | CLN00876790 | 27480441_1-68 | 0.59 | 1-29 | 30-68 |
| HG1023466 | CLN00884956 | NP_001786_1-593 | 1 | 1-16 | 17-593 |
| HG1023467 | CLN00905128 | NP_001842_1-23_ECOR1_13477273_104-886 | 0.97 | 1-23 | 24-808 |
| HG1023468 | CLN00875342 | NP_001842_1-23_ECOR1_11596170_71-460 | 0.96 | 1-23 | 24-416 |
| HG1023469 | CLN00933977 | 18569328 | 0.95 | 1-21 | 22-162 |
| HG1023470 | CLN00874475 | NP_005526_1-495 | 1 | 1-23 | 24-495 |
| HG1023471 | CLN00923348 | 39645305_1-526_17939658_233-464_C237S | 0.98 | 1-26 | 27-763 |
| HG1023472 | CLN00625649 | NP_059429_1-221 | 0.98 | 1-23 | 24-221 |
| HG1023473 | CLN00904832 | NP_001842_1-23_ECOR1_6832905_18-130 | 0.88 | 1-23 | 24-140 |
| HG1023474 | CLN00822755 | CLN00131297 | 0.98 | 1-34 | 35-240 |
| HG1023475 | CLN00736434 | CLN00144017.a | 0.67 | 1-20 | 21-85 |
| HG1023476 | CLN00938770 | 37183162_1-579 | 0.98 | 1-22 | 23-579 |
| HG1023477 | CLN00874765 | 298_aa.genscan | 0.96 | 1-15 | 16-131 |
| HG1023478 | CLN00770902 | 37541459 | 0.99 | 1-24 | 25-272 |
| HG1023479 | CLN00860791 | 34529527 | 0.87 | 1-18 | 19-140 |
| HG1023480 | CLN00905066 | NP_001842_1-23_ECOR1_NP_001770_28-233 | 0.9 | 1-23 | 24-232 |
| HG1023481 | CLN00875352 | NP_001842_1-23_ECOR1_17486578_61-129 | 0.93 | 1-23 | 24-95 |
| HG1023482 | CLN00942081 | 55665166_1-360 | 0.06 | 1-36 | 37-360 |
| HG1023483 | CLN00872016 | NP_001842_1-23_ECOR1_25989486_69-398 | 0.96 | 1-23 | 24-356 |
| HG1023484 | CLN00933988 | 21753982 | 0.82 | 1-20 | 21-183 |
| HG1023485 | CLN00938850 | 22044017_1-575 | 0.54 | 1-36 | 37-575 |
| HG1023486 | CLN00878190 | NP_057723_1-80 | 0.86 | 1-24 | 25-80 |
| HG1023487 | CLN00898948 | proteinkinase270A_1-403 | 0.82 | 1-33 | 34-403 |
| HG1023488 | CLN00920019 | NP_055695_1-170 | 1 | 1-19 | 20-170 |
| HG1023489 | CLN00938858 | 1890660_1-746 | 0.9 | 1-18 | 19-746 |
| HG1023490 | CLN00877039 | 16550253_1-105 | 0.32 | 1-29 | 30-105 |
| HG1023491 | CLN00938643 | NP_478062 | 0.68 | 1-24 | 25-58 |
| HG1023492 | CLN00849492 | 22047619 | 0.86 | 1-19 | 20-235 |
| HG1023493 | CLN00912948 | NP_001842_1-23_ECOR1_NP_612445_37-100 | 0.95 | 1-23 | 24-89 |
| HG1023494 | CLN00822056 | CLN00493523 | 0.89 | 1-26 | 27-189 |
| HG1023495 | CLN00897720 | 20140433_1-639 | 0.79 | 1-20 | 21-639 |
| HG1023496 | CLN00865318 | CLN00042589 | 0.61 | 1-32 | 33-138 |
| HG1023497 | CLN00940208 | NP_076927_71-454 | 0 | NA | NA |
| HG1023498 | CLN00860886 | 34527995 | 0.88 | 1-15 | 16-129 |
| HG1023499 | CLN00861035 | 21756729 | 0.88 | 1-33 | 34-128 |
| HG1023500 | CLN00923155 | 22046814 | 0.48 | 1-29 | 30-104 |
| HG1023501 | CLN00850182 | CLN00223180.a | 0.51 | 1-35 | 36-68 |
| HG1023502 | CLN00849356 | CLN00139795.a | 0.09 | 1-20 | 21-76 |
| HG1023503 | CLN00625745 | CLN00129208 | 0.2 | 1-23 | 24-193 |
| HG1023504 | CLN00625748 | CLN00540586 | 0 | NA | NA |
| HG1023505 | CLN00938585 | 51476585_1-855 | 0.94 | 1-22 | 23-855 |
| HG1023506 | CLN00733775 | CLN00232956 | 0.98 | 1-28 | 29-215 |
| HG1023507 | CLN00871450 | NP_001842_1-23_ECOR1_13124597_78-240 | 0.92 | 1-23 | 24-189 |
| HG1023508 | CLN00904865 | NP_001842_1-23_ECOR1_NP_055580_29-145 | 0.97 | 1-23 | 24-142 |
| HG1023509 | CLN00916449 | 20536316 | 0.77 | 1-25 | 26-183 |
| HG1023510 | CLN00878644 | NP_068702_1-35 | 0.99 | 1-20 | 21-35 |
| HG1023511 | CLN00658098 | NP_085049 | 1 | 1-26 | 27-116 |
| HG1023512 | CLN00914055 | 2587058_1-413 | 1 | 1-17 | 18-413 |
| HG1023513 | CLN00869032 | 31873423 | 0.34 | 1-16 | 17-147 |
| HG1023514 | CLN00874079 | NP_001842_1-23_ECOR1_NP_006219_97-126 | 0.97 | 1-23 | 24-57 |
| HG1023515 | CLN00913183 | NP_001842_1-23_ECOR1_50949569_47-148 | 0.92 | 1-23 | 24-127 |
| HG1023516 | CLN00877127 | 204_aa.genscan | 0.84 | 1-26 | 27-118 |
| HG1023517 | CLN00875524 | NP_001B42_1-23_ECOR1_NP_631906_187-299 | 0.93 | 1-23 | 24-139 |
| HG1023518 | CLN00942038 | 51468555 | 0.8 | 1-20 | 21-212 |
| HG1023519 | CLN00852080 | CLN00161892 | 0 | NA | NA |
| HG1023520 | CLN00923451 | NP_001648_1-183 | 0.97 | 1-21 | 22-183 |
| HG1023521 | CLN00849297 | 34365357 | 0.88 | 1-15 | 16-219 |
| HG1023522 | CLN00869329 | NP_060159 | 0.63 | 1-19 | 20-141 |
| HG1023523 | CLN00919867 | NP_001057_1-257 | 0.89 | 1-22 | 23-257 |
| HG1023524 | CLN00865330 | CLN00455998 | 0.88 | 1-17 | 18-98 |
| HG1023525 | CLN00899057 | NP_037363_1-457 | 0.42 | 1-31 | 32-457 |
| HG1023526 | CLN00929996 | 34531092 | 0.98 | 1-15 | 16-164 |
| HG1023527 | CLN00921353 | 34533908 | 0.91 | 1-25 | 26-130 |
| HG1023528 | CLN00885080 | 10435604_1-385 | 0.96 | 1-25 | 26-385 |
| HG1023529 | CLN00870230 | 30584491_1-189 | 1 | 1-16 | 17-189 |
| HG1023530 | CLN00542945 | NP_689669 | 0.92 | 1-20 | 21-242 |
| HG1023531 | CLN00800085 | CLN00519958 | 0.99 | 1-23 | 24-99 |
| HG1023532 | CLN00849377 | CLN00516788.a | 0.92 | 1-26 | 27-71 |
| HG1023533 | CLN00870168 | NP_001056_1-205 | 0.94 | 1-21 | 22-205 |
| HG1023534 | CLN00870451 | NP_006685_1-92 | 0.84 | 1-30 | 31-92 |
| HG1023535 | CLN00874596 | NP_003719_1-284 | 0.27 | 1-35 | 36-284 |
| HG1023536 | CLN00871329 | NP_001842_1-23_ECOR1_NP_000585_75-233 | 0.93 | 1-23 | 24-185 |
| HG1023537 | CLN00928403 | NP_001842_1-23_ECOR1_NP_001004419.1_57-194 | 0.93 | 1-23 | 24-163 |
| HG1023538 | CLN00885012 | 902797_1-320 | 0.98 | 1-17 | 18-320 |
| HG1023539 | CLN00874304 | NP_001842_1-23_ECOR1_20141490_24-55 | 0.91 | 1-23 | 24-59 |
| HG1023540 | CLN00773284 | NP_001842_1-23_ECOR1_CLN00489263_113-206 | 0.9 | 1-23 | 24-119 |
| HG1023541 | CLN00876990 | 314_aa.genscan | 0.75 | 1-18 | 19-133 |
| HG1023542 | CLN00869121 | 13097804 | 0.09 | 1-37 | 38-187 |
| HG1023543 | CLN00554611 | NP_079254 | 0.74 | 1-29 | 30-191 |
| HG1023544 | CLN00932074 | 7959199_1-410 | 0.41 | 1-28 | 29-410 |
| HG1023545 | CLN00878231 | NP_653087_1-238 | 0.98 | 1-27 | 28-238 |
| HG1023546 | CLN00921368 | 27498551 | 0.63 | 1-24 | 25-660 |
| HG1023547 | CLN00874814 | NP_757351_1-446 | 0.95 | 1-30 | 31-446 |
| HG1023548 | CLN00859148 | CLN00173156 | 1 | 1-20 | 21-61 |
| HG1023549 | CLN00870314 | NP_780778_1-130 | 1 | 1-21 | 22-130 |
| HG1023550 | CLN00823103 | NP_060330 | 1 | 1-22 | 23-341 |
| HG1023551 | CLN00872067 | NP_001842_1-23_ECOR1_NP_060615_138-470 | 0.88 | 1-23 | 24-359 |
| HG1023552 | CLN00860841 | 27482797 | 0.76 | 1-25 | 26-128 |
| HG1023553 | CLN00860842 | 27483988 | 0.9 | 1-25 | 26-150 |
| HG1023554 | CLN00927329 | NP_002498_1-249 | 0.98 | 1-26 | 27-249 |
| HG1023555 | CLN00773292 | NP_001842_1-23_ECOR1_NP_006841_49-206 | 0.93 | 1-23 | 24-183 |
| HG1023556 | CLN00733878 | CLN00271203 | 0.99 | 1-32 | 33-228 |
| HG1023557 | CLN00919453 | 13279053 | 0.99 | 1-31 | 32-253 |
| HG1023558 | CLN00658120 | 7106808 | 0.71 | 1-23 | 24-144 |
| HG1023559 | CLN00546831 | NP_005746 | 0.99 | 1-20 | 21-229 |
| HG1023560 | CLN00933908 | 29742396 | 0.98 | 1-23 | 24-121 |
| HG1023561 | CLN00929968 | 37181600 | 0.99 | 1-23 | 24-91 |
| HG1023562 | CLN00603883 | 19338684 | 0.8 | 1-23 | 24-229 |
| HG1023563 | CLN00795173 | NP_003815 | 0.97 | 1-16 | 17-208 |
| HG1023564 | CLN00851780 | 37183032 | 0.96 | 1-15 | 16-109 |
| HG1023565 | CLN00528436 | 22044951 | 0.98 | 1-21 | 22-950 |
| HG1023566 | CLN00853320 | 7770125 | 0.74 | 1-25 | 26-68 |

TABLE 6
Examples of human demyelinating pathologies

```
  Brain injuries (e.g. injuries caused by a brain surgery or physical
  accidents)
Spinal cord injuries
Aging
  Alzheimer's disease
  Multiple sclerosis (MS)
  Macular degeneration
  Glaucoma
  Diabetic retinopathy
  Peripheral neuropathy
  Huntington's disease
  Amyotrophic lateral sclerosis
Parkinson's disease
Zellweger syndrome
  Stroke (e.g. hemorrhagic stroke, focal ischemic stroke or global
  ischemic stroke)
Adrenoleukodystrophy
Cockayne's syndrome
Orthochromatic leukodystrophy
  Depression
  Epilepsy
  Neurosis and psychosis. Other examples include
  Idiopathic demyelinating polyneuropathy
  Leukodystrophy
  Canavan's disease
  Optic neuritis
  Schizophrenia
  Transverse myelitis
  Progressive multifocal leukoencephalopathy
  Infection induced leukoencephalopathies
  Toxin, treatment or chemotherapy induced demyelination
  Guillain-Barre syndrome
  Acute disseminated encephalomyelitis
Alexander's disease
Childhood ataxia with CNS hypomyelination (vanishing-white-matter
disease)
Cerebrotendinous xanthomatosis
Krabbe disease (globoid cell leukodystrophy)
Metachromatic leukodystrophy
Refsum disease
Pelizaeus-Merzbacher disease
Phenylketouria
Chronic inflammatory demyelinating polyneuropathy
Charcot-Marie-Tooth disease
Microcephaly, failure to thrive, and kyphoscoliosis
Post-infectious encephalitis, acute disseminated encephalomyelitis
Multifocal motor neuropathy
Multifocal acquired sensory and motor neuropathy
Anti-myelin-associated-glycoprotein (anti-MAG) neuropathies
Demyelinating meuropathies associated with momoclonal gammopathies
Polyneuropathy, organomegaly, endocrinopaty, monoclomal gammopathy
and skin changes
Polyneuropathy associated with MS
Multifocal sensorimotor demyelinating neuropathy
Neuropathies assocaited with monoclonal gammopathies
Central pontine myelinolysis
Extra pontine myelinolysis
Marchiafava-Ginami disease
```

1. An isolated nucleic acid molecule comprising a first polynucleotide sequence of any one of: (A) SEQ. ID. NOS.: 122, 135, 137, 161-162, 176, 192 or 201; (B) a polynucleotide sequence encoding a polypeptide of SEQ. ID. NOS.: 9, 22, 24, 48-49, 63, 79, or 88 (C) biologically active fragments thereof; or (D) complements thereof.


SEQUENCE LISTING (226 sequences)

SEQ ID NO: 1 - protein, 201 aa, Homo sapiens
MKTCWKIPVFFFVCSFLEPWASAEFVTFRIFQTCDEKKFQLPENFTELSCYNYGSGSVKNCCPLNWEYFQSSCYFFSTDTISWALSLKNCSAMGAHLVVINSQEEQEFLSYKKPKMREFFIGLSDQVVEGQWQWVDGTPLTKSLSFWDVGEPNNIATLEDCATMRDSSNPRQNWNDVTCFLNYFRICEMVGINPLNKGKSL

SEQ ID NO: 2 - protein, 124 aa, Homo sapiens
MACHTLLVCALLVSLLPSCLPFLCWRLIPFMLSPPRCLHCLLGQSYLVPSLFKKQALPIMVEFLSIKGHTFRPPGTSREQCCAQETEFSCVGPAAAIRFQTPTEITKTALCNLEAYIIILSGIK

SEQ ID NO: 3 - protein, 236 aa, Homo sapiens
MNGLSLSELCCLFCCPPCPGRIAAKLAFLPPEATYSLVPEPEPGPGGAGAAPLGTLRASSGAPGRWKLHLTERADFQYSQRELDTIEVFPTKSARGNHVSCMYVRCVPGARYTVLFSHGNAVDLGQMSSFYIGLGSRLHCNIFSYDYSGYGASSGRPSERNLYADIDAAWQALRTRYGISPDSIILYGQSIGTVPTVDLASRYECAAVVLHSPLTSGMRVAFPDTKKTYCFDAFPK

SEQ ID NO: 4 - protein, 189 aa, Homo sapiens [sequence omitted]

SEQ ID NO: 5 - protein, 185 aa, Homo sapiens
MKTCWKIPVFFFVCSFLEPWASAEFEFRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 6 - protein, 78 aa, Homo sapiens
MAASAALSAAAAAAALSGLAVRLSRSAAARGSYGAFCKGLTRTLLTFFDLAWRLRMNFPYFYIVASVMLNVRLQVRIE

SEQ ID NO: 7 - protein, 188 aa, Homo sapiens
MAPPWAGGERRGPGTTCLHSPWMLEAAPPWAGGEGRELGAACLHSLRMLEAGGSEAATARGRGDFGAASCSDLAFRCASSQNPRSLEPVASSPERRRRQPSRAFACTLPGCWRLEAVTQQQREATLERPHIATSPSAARPPRAQEARNLWHPSLKGEDGNPAEALLGWGLHDRAGLIKWRPTRGSNPG

SEQ ID NO: 8 - protein, 218 aa, Homo sapiens
MAVGTRGTLLKKSLTVWFCGPGARSATRAVSTSLPRREQVTWCCCSGSWPRRTASTSWRCSMAANTFYFRPNGAGDTRQNLIPDFYAFRVINNGKVADIKKVNNFIREQDLYALKSVKIPVRNHGILMETHKELKPLLSPSSETTVTVELPEADRAGAGTGAQAGQLMGFFKGIDQDIERAVQSEIFLHESYCMDTSHQPLLPAPPKTPMDGADCGIQ

SEQ ID NO: 9 - protein, 70 aa, Homo sapiens [sequence omitted]

SEQ ID NO: 10 - protein, 598 aa, Homo sapiens
MQAAVAVSVPFLLLCVLGTCPPARCGQAGDASLMELEKRKENRFVERQSIVPLRLIYRSGGEDESRHDALDTRVRGDLGGPQLTHVDQASFQVDAFGTSFILDVVLNHDLLSSEYIERHIEHGGKTVEVKGGEHCYYQGHIRGNPDSFVALSTCHGLHGMFYDGNHTYLIEPEENDTTQEDFHFHSVYKSRLFEFSLDDLPSEFQQVNITPSKFILKPRPKRSKRQLRRYPRNVEEETKYIELMIVNDHLMFKKHRLSVVHTNTYAKSVVNMADLIYKDQLKTRIVLVAMETWATDNKFAISENPLITLREFMKYRRDFIKEKSDAVHLFSGSQFESSRSGAAYIGGICSLLKGGGVNEFGKTDLMAVTLAQSLAHNIGIISDKRKLASGECKCEDTWSGCIMGDTGYYLPKKFTQCNIEEYHDFLNSGGGACLFNKPSKLLDPPECGNGFIETGEECDCGTPAECVLEGAECCKKCTLTQDSQCSDGLCCKKCKFQPMGTVCREAVNDCDIRETCSGNSSQCAPNIHKMDGYSCDGVQGICFGGRCKTRDRQCKYIWGQKVTASDKYCYEKLNIEGTEKGNCGKDKDTWIQCNKRDV

SEQ ID NO: 11 - protein, 150 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDET

SEQ ID NO: 12 - protein, 68 aa, Homo sapiens
MRDQLILILSPAGIVSIFSLAALTCESWCLLIALICMSLMTNDAEHLSVCSFFSPLFNRILCFLTVEF

SEQ ID NO: 13 - protein, 593 aa, Homo sapiens
MQRLMMLLATSGACLGLLAVAAVAAAGANPAQRDTHSLLPTHRRQKRDWIWNQMHIDEEKNTSLPHHVGKIKSSVSRKNAKYLLKGEYVGKVFRVDAETGDVFAIERLDRENISEYHLTAVIVDKDTGENLETPSSFTIKVHDVNDNWPVFTHRLFNASVPESSAVGTSVISVTAVDADDPTVGDHASVMYQILKGKEYFAIDNSGRIITITKSLDREKQARYEIVVEARDAQGLRGDSGTATVLVTLQDINDNFPFFTQTKYTFVVPEDTRVGTSVGSLFVEDPDEPQNRMTKYSILRGDYQDAFTIETNPAHNEGIIKPMKPLDYEYIQQYSFIVEATDPTIDLRYMSPPAGNRAQVIINITDVDEPPIFQQPFYHFQLKENQKKPLIGTVLAMDPDAARHSIGYSIRRTSDKGQFFRVTKKGDIYNEKELDREVYPWYNLTVEAKELDSTGTPTGKESIVQVHIEVLDENDNAPEFAKPYQPKVCENAVHGQLVLQISAIDKDITPRNVKFKFTLNTENNFTLTDNHDNTANITVKYGQFDREHTKVHFLPVVISDNGMPSRTGTSTLTVAVCKCNEQGEFTFCEDMAAQ

SEQ ID NO: 14 - protein, 808 aa, Homo sapiens
MKTCWKIPVFFFVCSFLEPWASAEFRHRPSLLLENYQPWLDLKISSKVDASLSEVDIPSIITKKLLKAAMKHIEVIVKARQKVKNTEFLQQAALEEYGPELHVALRSRRDELHYLRKLTELLFPYILPPKATDCRSLTLLIREILSGSVFLPSLDFLADPDTVNHLLIIFIDDSPPEKATEPASPLVPFLQKFAEPRNKKPSVLKLELKQIREQQDLLFRFMNFLKQEGAVHVLQFCLTVEEFNDRILRPELSNDEMLSLHEELQKIYKTYCLDESIDKIRFDPFIVEEIQRIAEGPYIDVVKLQTMRCLFEAYEHVLSLLENVFTPMFCHSDEYFRQLLRGAESPTRNSKLNRNTQKRGESFGISRIGSKIKGVFKSTTMEGAMLPNYGVAEGEDDFIEEGIVVMEDDSPVEAVSTPNTPRNLAAWKISIPYVDFFEDPSSERKEKKERIPVFCIDVERNDRRAVGHEPEHWSVYRRYLEFYVLESKLTEFHGAFPDAQLPSKRIIGPKNYEFLKSKREEFQEYLQKLLQHPELSNSQLLADFLSPNGGETQFLDKILPDVNLGKIIKSVPGKLMKEKGQHLEPFIMNFINSCESPKPKPSRPELTILSPTSENNKKLFNDLFKNNANRAENTERKQNQNYFMEVMTVEGVYDYLMYVGRVVFQVPDWLHHLLMGTRILFKNTLEMYTDYYLQCKLEQLFQEHRLVSLITLLRDAIFCENTEPRSLQDKQKGAKQTFEEMMNYIPDLLVKCIGEETKYESIRLLFDGLQQPVLNKQLTYVLLDIVIQELFPELNKVQKEVTSVTSWM

SEQ ID NO: 15 - protein, 416 aa, Homo sapiens
MKTCWKIPVFFFVCSFLEPWASAEFEFEVISRGFTLVSAACPFNKAGQHPSQHLIGLRKAVYRTLRANFQAARLATLYMLKNYPLNSESDNVTNYICVVPFKELGLGLSEEQISEEEAHNFTDGFSLPALKVLFQLWVAQSSEFFRRLALLLSTANSPPGPLLTPALLPHRILSDVTQGLPHAHSACLEELKRSYEFYRYFETQHQSVPQCLSKTQQKSRELNNVHTAVRSLQLHLKALLNEVIILEDELEKLVCTKETQELVSEAYPILEQKLKLIQPHVQASNNCWEEAISQIDKLLRRNTDKKGKPEIACENPHCTVVPLKQPTLHIADKDPIPEEQELEAYVDDIDIDSDFRKDDFYYLSQEDKERQKREHEESKRVLQELKSVLGFKASEAERQKWKQLLFSDHGVKSAWN

SEQ ID NO: 16 - protein, 162 aa, Homo sapiens
MGTKLQSWTFWLCLLCNGIRSTGLGRQLDNGDRMSGQSWFHSMLVNELSPGSLQSSGPQLTVELEVPQHPPLPPSRAPLLLLNGERMEPTLETRDLAKWVGQEVPGKRQTELQNAWLLLLNGWGERDTACDGTDKPVQIALDSLTDTVSLKHIAHLFLFVIL

SEQ ID NO: 17 - protein, 495 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSE

SEQ ID NO: 18 - protein, 763 aa, Homo sapiens
AATMGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHPRDRSTVEGRVTFAKVEETIAVRCLAKNLLGAENRELKLVAPTLRSELTGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 221 aa, Homo sapiens
MSGSSLPSALALSLLLVSGSLLPGPGAAQNEPRIVTSEEVIIRDSPVLPVTLQCNLTSSSHTLTYSYWTKNGVELSATRKNASNMEYRINKPRAEDSGEYHCVYHFVSAPKANATIEVKAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHL

SEQ ID NO: 20 - protein, 140 aa, Homo sapiens
MKTCWKIPVFFFVCSFLEPWASAEFEFLPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVME

SEQ ID NO: 21 - protein, 240 aa, Homo sapiens
MGPLPAPSCTQRITWKGLLLTASLLNFWNPPTTAEVTIEAQPPKVSEGKDVLLLVHNLPQNLPGYFWYKGEMTDLYHYIISYIVDGKIIIYGPAYSGRETVYSNASLLIQNVTRKDAGTYTLHIIKRGDETREEIRHFTFTLYYGPDLPRIYPSFTYYRSGENLDLSCFTESNPPAEYFWTINGKFQQSGQKLFIPQITRNHSGLYACSVHNSATGKEISKSMTVKVSGPCHGDLTESQS

SEQ ID NO: 22 - protein, 85 aa, Homo sapiens [sequence omitted]

SEQ ID NO: 23 - protein, 579 aa, Homo sapiens
MRTPQLALLQVFFLVFPDGVRPQPSSSPSGAVPTSLELQRGTDGGTLQSPSEATATRPAVPGLPTVVPTLVTPSAPGNRTVDLFPVLPICVCDLTPGACDINCCCDRDCYLLHPRTVFSFCLPGSVRSSSWVCVDNSVIFRSNSPFPSRVFMDSNGIRQFCVHVNNSNLNYFQKLQKVNATNFQALAAEFGGESFTSTFQTQSPPSFYRAGDPILTYFPKWSVISLLRQPAGVGAGGLCAESNPAGFLESKSTTCTRFFKNLASSCTLDSALNAASYYNFTVLKVPRSMTDPQNMEFQVPVILTSQANAPLLAGNTCQNVVSQVTYEIETNGTFGIQKVSVSLGQTNLTVEPGASLQQHFILRFRAFQQSTAASLTSPRSGNPGYIVGKPLLALTDDISYSMTLLQSQGNGSCSVKRHEVQFGVNAISGCKLRLKKADCSHLQQEIYQTLHGRPRPEYVAIFGNADPAQKGGWTRILNRHCSISAINCTSCCLIPVSLEIQVLWAYVGLLSNPQAHVSGVRFLYQCQSIQDSQQVTEVSLTTLVNFVDITQKPQPPRGQPKMDWKWPFDFFPFKVAFSR

SEQ ID NO: 24 - protein, 131 aa, Homo sapiens [sequence omitted]

SEQ ID NO: 25 - protein, 272 aa, Homo sapiens
MPRKQPAGCIFLLTFLGLSGLVGTVTRTYYIGIVEEYWNYVPQGKNVITGKSFTEDKLATLFLERGPNRIGSIYKKAVYRRFTDGTYSIEIPKPPWLGFLGPILRAEVGDVIVIHLKNFASRPYSLHPHGVFYNKDSEGALYPDGTSGRNKNDDMVPPGKNYTYVWPVREEYAPTPADANCLTWVYHSHIDAPKDICSGLIGPLLVCKEGILNRYSGTRNDVDREFVIMFTLVDENQSWYLNENIKHFCTNPDSVDKKDAVFQRSNKMHELF

SEQ ID NO: 26 - protein, 140 aa, Homo sapiens
MLGISAIGGLCCLHRLYSCIPGEGGRAAKEKSQSQQPALRPSKLCTGGLGSALWARGAGKRAGRGTAATSARAAQEAPPYTALQVPQREQPMLPKHPLHTHQGSWRREVPQGKAPSPRRASGPGLLWLPTAIISIYRAQS

SEQ ID NO: 27 - protein, 232 aa, Homo sapiens
MKTCWKIPVFFFVCSFLEPWASAEFEFFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLY

SEQ ID NO: 28 - protein, 95 aa, Homo sapiens
MKTCWKIPVFFFVCSFLEPWASAEFEFAPSIIAAQRQTILAEYNMSCDDTGKLILKPRPHVQCQSSLIAIGRKTALLRISDTAKSHKGFLLQLDM

SEQ ID NO: 29 - protein, 360 aa, Homo sapiens
MSEGAAAASPPGAASAAAASAEEGTAAAAAAAAAGGGPDGGGEGAAEPPRELRCSDCIVWNRQQTWLCVVPLFIGFIGLGLSLMLLKWIVVGSVKEYVPTDLVDSKGMGQDPFFLSKPSSFPKAMETTTTTTSTTSPATPSAGGAASSRTPNRISTRLTTITRAPTRFPGHRVPIRASPRSTTARNTAAPATVPSTTAPFFSSSTLGSRPPVPGTPSTQAMPSWPTAAYATSSYLHDSTPSWTLSPFQDAASSSSSSSSSATTTTPETSTSPKFHTTTYSTERSEHFKPCRDKDLAYCLNDGECFVIETLTGSHKHCRCKEGYQGVRCDQFLPKTDSILSDPTDHLGIEFMESEEVYQRQ

SEQ ID NO: 30 - protein, 356 aa, Homo sapiens
MKTCWKIPVFFFVCSFLEPWASAEFEFPYLRVTVMPLLTLPQTRQRAKNIYDILPWRQEDLGRHESRSMRIFSTESLLSRNSESPEHVPSQAGNAFQEHTAHIHATEYAVGIYDNAMVPQMCGNLTPSAHCINVRASRDCASISSEDSHDYVNVPTAEEIAETLASTKSPSRNLFVLPSTQKLEFTEERDEGCGDAGDCTSLYSPGAEDSDSLSNGEGSSQISNDYVNMTGLDLSAIQERQLWVAFQCCRDYENVPAADPSGSQQQAEKDVPSSNIGHVEDKTDDPGTHVQCVKRTFLASGDYADFQPFTQSEDSQMKHREEMSNEDSSDYENVLTAKLGGRDSEQGPGTQLLPDE

SEQ ID NO: 31 - protein, 183 aa, Homo sapiens
MVRIWTTIMIVLILLLRIGPNKPSLSGRQAPAQAQTSDLVPSLFPLGLWAPGFCTWSSPDEDKRPRKPVPGTGNRDSGTRRRLQDATEQDPRPGNDVASAETAGPPSPSGIRAQDRAPRHRRAPPARMPVAPAPSADGEPLQEQGGGLFHRTRSVYYGLELNTWMKVERLFVEKFHQSFSLDN

SEQ ID NO: 32 - protein, 575 aa, Homo sapiens
MTAEGPSPPARWHRRLPGLWAAALLLLGLPRLSVRADGKFFVLESQNGSQGLQLEAARLSCKSRGAHLASADELRRVVQDCSFAVCTTGWLADGTLGTTVCSKGSGEQQIMRAVDVRIESNPVPGGTYSALCIKDEEKPCGDPPSFPHTILQGRTGLEMGDELLYVCAPGHIMGHRETAFTLLCNSCGEWYGLVQACGKDEAEAHIDYEDNFPDDRSVSFRELMEDSRTEADEDRGQGDSSEEAPKQDRLVSISVGRENIARDKVFVPTTGLPGAGSSVPADSPGSRLLQKHLFWFPAEAFHKPGLEKEVDDDTKKQFSAGDNHSGVKLVPGEPETKVIYGNTDGPSGPFVGKNDSKAGDPVVSSSDESWLDGYPVTEGAWRKTEAEEEEDGDRGDGSVGLDENVLVTPDQPILVEVKKPKSSTLTPSEGMTHSSVLPSQMLDVEALALRPVNASETEGIGDGDLTKYQSTLPWRFITEESPMATLSYELTSSTLEILTVNTVKQTPNHIPSTIMATTQPPVETTVPEIQDSFPYLLSEDFFGQEGPGPGASEELHPTLESCVGDGCPGLSRGPV

SEQ ID NO: 33 - protein, 80 aa, Homo sapiens
MARGSLRRLLRLLVLGLWLALLRSVAGEQAPGTAPCSRGSSWSADLDKCMDCASCRARPHSDFCLGCAAAPPAPFRLLWP

SEQ ID NO: 34 - protein, 403 aa, Homo sapiens
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRAPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMG

SEQ ID NO: 35 - protein, 170 aa, Homo sapiens
MLRAALPALLLPLLGLAAAAVADCPSSTWIQFQDSCYIFLQEAIKVESIEDVRNQCTDHGADMISIHNEEENAFILDTLKKQWKGPDDILLGMFYDTDDASFKWFDNSNMTFDKWTDQDDDEDLVDTCAFLHIKTGEWKKGNCEVSSVEGTLCKTAIPYKRKYLSDNHIL

SEQ ID NO: 36 - protein, 746 aa, Homo sapiens
MARAQALVLALTFQLCAPETETPAAGCTFEEASDPAVPCEYSQAQYDDFQWEQVRIHPGTRAPADLPHGSYLMVNTSQHAPGQRAHVIFQSLSENDTHCVQFSYFLYSRDGHSPGTLGVYVRVNGGPLGSAVWNMTGSHGRQWHQAELAVSTFWPNEYQVLFEALISPDRRGYMGLDDILLLSYPCAKAPHFSRLGDVEVNAGQNASFQCMAAGRAAEAERFLLQRQSGALVPAAGVRHISHRRFLATFPLAAVSRAEQDLYRCVSQAPRGAGVSNFAELIVKEPPTPIAPPQLLRAGPTYLIIQLNTNSIIGDGPIVRKEIEYRMARGPWAEVHAVSLQTYKLWHLDPDTEYEISVLSRPGDGGTGRPGPPLISRTKCAEPMRAPKGLAFAEIQARQLALQWEPLGYNVTRCHTYTVSLCYHYTLGSSHNQTIRECVKTEQGVSRYTIKNLLPYRNVHVRLVLTNPEGRKEGKEVTFQTDEDVPSGIAAESLTFTPLEDMIFLKWEEPQEPNGLITQYEISYQSIESSDPAVNVPGPRRTISKLRNETYHVFSNLHPGTTYLFSVRARTGKGFGQAALTEITTNISAPSFDYADMPSPLGESENTITVLLRPAQGRGAPISVYQVIVEEERARRLRREPGGQDCFPVPLTFEAALARGLVHYFGAELAASSLPEAMPFTVGDNQTYRGFWNPPLEPRKAYLIYFQAASHLKGETRLNCIRIARKAACKESKRPLEVSQRSEEMGL

SEQ ID NO: 37 - protein, 105 aa, Homo sapiens
MPFPVRVEVDMVRVMEVFLAQLRLLFGIAQPQLPPKCLLSGPTSEGLMTWELDRLLWARSVENLATATTTLTSLAQLLGKISNIVIKDDVASEVYKAVAAVQKSA

SEQ ID NO: 38 - protein, 58 aa, Homo sapiens
MNWKVLEHVPLLLYILAAKTLILCLTFAGVKMYQRKRLEAKQQKLEAERKKQSEKKDN

SEQ ID NO: 39 - protein, 235 aa, Homo sapiens
MKYVFYLGVLAGTFFFADSSVQKEDPAPYLVYLKSHFNPCVGVLIKPSWVLAPAHCYLPNLKVMLGNFKSRVRDGTEQTINPIQIVRYWNYSHSAPQDDLMLIKLAKPAMLNPKVQPLTLATTNVRPGTVCLLSGLDWSQENSGRHPDLRQNLEAPVMSDRECQKTEQGKSHRNSLCVKFVKVFSRIFGEVAVATVICKDKLQGIEVGHFMGGDVGIYTNVYKYVSWIENTAKDK

SEQ ID NO: 40 - protein, 89 aa, Homo sapiens
MKTCWKIPVFFFVCSFLEPWASAEFKRNKRRIMRIFSVPPTEETLSEPNFYDTISKIRLRQQLEMYSISRKYDYQQPQNQADSVQLSLE

SEQ ID NO: 41 - protein, 189 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 42 - protein, 639 aa, Homo sapiens
MGDHLDLLLGVVLMAGPVFGIPSCSFDGRIAFYRFCNLTQVPQVLNTTERLLLSFNYIRTVTASSFPFLEQLQLLELGSQYTPLTIDKEAFRNLPNLRILDLGSSKIYFLHPDAFQGLFHLFELRLYFCGLSDAVLKDGYFRNLKALTRLDLSKNQIRSLYLHPSFGKLNSLKSIDFSSNQIFLVCEHELEPLQGKTLSFFSLAANSLYSRVSVDWGKCMNPFRNMVLEIVDVSGNGWTVDITGNFSNAISKSQAFSLILAHHIMGAGFGFHNIKDPDQNTFAGLARSSVRHLDLSHGFVFSLNSRVFETLKDLKVLNLAYNKINKIADEAFYGLDNLQVLNLSYNLLGELCSSNFYGLPKVAYIDLQKNHIAIIQDQTFKFLEKLQTLDLRDNALTTIHFIPSIPDIFLSGNKLVTLPKINLTANLIHLSENRLENLDILYFLLRVPHLQILILNQNRFSSCSGDQTPSENPSLEQLFLGENMLQLAWETELCWDVFEGLSHLQVLYLNHNYLNSLPPGVFSHLTALRGLSLNSNRLTVLSHNDLPANLEILDISRNQLLAPNPDVFVSLSVLDITHNKFICECELSTFINWLNHTNVTIAGPPADIYCVYPDSLSGVSLFSLSTEGCDEEEVLKSLK

SEQ ID NO: 43 - protein, 138 aa, Homo sapiens
MAASRWARKAVVLLCASDLLLLLLLLPPPGSCAAEGSPGTPDESTPPPRKKKKDIRDYNDADMARLLEQWETPEPLPVLPEVPSTCACLSSASLIWTCFSHLSPHALVKRVWPPAKQGLGGKESPASAWLPHRGGELK

SEQ ID NO: 44 - protein, 384 aa, Homo sapiens
FDCSGKYRCRSSFKCIELIARCDGVSDCKDGEDEYRCVRVGGQNAVLQVFTAASWKTMCSDDWKGHYANVACAQLGFPSYVSSDNLRVSSLEGQFREEFVSIDHLLPDDKVTALHHSVYVREGCASGHVVTLQCTACGHRRGYSSRIVGGNMSLLSQWPWQASLQFQGYHLCGGSVITPLWIITAAHCVYDLYLPKSWTIQVGLVSLLDNPAPSHLVEKIVYHSKYKPKRLGNDIALMKLAGPLTFNEMIQPVCLPNSEENFPDGKVCWTSGWGATEDGAGDASPVLNHAAVPLISNKICNHRDVYGGIISPSMLCAGYLTGGVDSCQGDSGGPLVCQERRLWKLVGATSFGIGCAEVNKPGVYTRVTSFLDWIHEQMERDLKT

SEQ ID NO: 45 - protein, 129 aa, Homo sapiens
MRSLLILSWDSWSIAFLAGNLCGQWHLCPSFAWASRSRHRCLLPARLQLDQVYCKQAASTAGTGEHGGGQKLGDTRNCRAPKRVSQACIRNLLGLGSLKGHSSSLLLSSLLLVTRNVASKGCVSALFVL

SEQ ID NO: 46 - protein, 128 aa, Homo sapiens
MLLAPILAMLTILSCLKCFMTLCFLSVPFSSLAGSFQSPSGAVLPLPDPSQFFFRVPNPGSPLLSVNTIPLVELNLRIRSVFVTQIFNERLLRDGFFLISTGDKNMHDCCISGLIGKNAKCAWNHESI

SEQ ID NO: 47 - protein, 104 aa, Homo sapiens
MSSLQRDLLGPPPPFSILFLSFIFFEALTHFQKLSGLLMCLSLDCLLPLLDCTLHKRAKLCLLHSLLSSQKQAACWRPSNTCEMKGHLVLFHLFPPGSQVASLH

SEQ ID NO: 48 - protein, 68 aa, Homo sapiens
MTCGLTLLITTTIISTRLSSYILLLIMVIHSFIHSQNIYDVFTLHQVPCQALGTQLWQSRQSICSFGA

SEQ ID NO: 49 - protein, 76 aa, Homo sapiens
MSRVSLRCCCGFWCWRRRGGGRVGAALRCGGPYLPGPGRSHCWGTCSCSPEAWTAHSIPDRNGGTEFWIREGGAGD

SEQ ID NO: 50 - protein, 193 aa, Homo sapiens
MSGSSLPSALALSLLLVSGSLLPGPGAAQNAAPDITGHKRSENKNEGQDATMYCKSVGYPHPDWIWRKKENGMPMDIVNTSGRFFIINKENYTELNIVNLQITEDPGEYECNATNAIGSASVVTVLRVRSHLAPLWPFLGILAEIIILVVIIVVYEKRKRPDEVPDDDEPAGPMKTNSTNNHKDKNLRQRNTN

SEQ ID NO: 51 - protein, 155 aa, Homo sapiens
MWQDPEVWVTLCQRKWLSYCSKNSRTNVSARIVKMERVQPLEENVGNAARPRFERNKLLLVASVIQGLGLLLCFTYICLHFSALQVSHRYPRIQSIKVQFTGLLITAFPALPSYKLSHRIVLRIKQNTNHGSQIMTFSRLVGKRDLPCLRRDYGF

SEQ ID NO: 52 - protein, 855 aa, Homo sapiens
MVISLNSCLSFICLLLCHWIGTASPLNLEDPNVCSHWESYSVTVQESYPHPFDQIYYTSCTDILNWFKCTRHRVSYRTAYRHGEKTMYRRKSQCCPGFYESGEMCVPHCADKCVHGRCIAPNTCQCEPGWGGTNCSSACDGDHWGPHCTSRCQCKNGALCNPITGACHCAAGFRGWRCEDRCEQGTYGNDCHQRCQCQNGATCDHVTGECRCPPGYTGAFCEDLCPPGKHGPQCEQRCPCQNGGVCHHVTGECSCPSGWMGTVCGQPCPEGRFGKNCSQECQCHNGGTCDAATGQCHCSPGYTGERCQDECPVGTYGVLCAETCQCVNGGKCYHVSGACLCEAGFAGERCEARLCPEGLYGIKCDKRCPCHLENTHSCHPMSGECACKPGWSGLYCNETCSPGFYGEACQQICSCQNGADCDSVTGKCTCAPGFKGIDCSTPCPLGTYGINCSSRCGCKNDAVCSPVDGSCTCKAGWHGVDCSIRCPSGTWGFGCNLTCQCLNGGACNTLDGTCTCAPGWRGEKCELPCQDGTYGLNCAERCGCSHADGCHPTTGHCRCLPGWSGVHCDSVCAEGRWGPNCSLPCYCKNGASCSPDDGICECAPGFRGTTCQRICSPGFYGHRCSQTCPQCVHSSGPCHHITGLCDCLPGFTGALCNEVCPSGRFGKNCAGICTCTNNGTCNPIDRSCQCYPGWIGSDCSQPCPPAHWGPNCIHTCNCHNGAFCSAYDGECKCTPGWTGLYCTQRCPLGFYGKDCALICQCQNGADCDHISGQCTCRTGFMGRHCEQKCPSGTYGYGCRQICDCLNNSTCDHITGTCYCSPGWKGARCDQAGVIIVGNLNSLSRTSTALPADSYQ

SEQ ID NO: 53 - protein, 215 aa, Homo sapiens
MDVGPSSLPHLGLKLLLLLLLLPLRGQANTGCYGIPGMPGLPGAPGKDGYDGLPGPKGEPGIPAIPGIRGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 54 - protein, 189 aa, Homo sapiens
MKTCWKIPVFFFVCSFLEPWASAEFEFSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 55 - protein, 142 aa, Homo sapiens
MKTCWKIPVFFFVCSFLEPWASAEFRSDPNFKNRLRERRKKQKLAKERAGLSKLPDLKDAEAVQKFFLEEIQLGEELLAQGEYEKGVDHLTNAIAVCGQPQQLLQVLQQTLPPPVFQMLLTKLPTISQRIVSAQSLAEDDVE

SEQ ID NO: 56 - protein, 183 aa, Homo sapiens
MLYKSSDRPAHKVSMLLLCHALAIAVVQIVIFSESWAFAKNINFYNVRPPLDPTPFPNSFKCFTCENAGDNYNCNRWAEDKWCPQNTQYCLTVHHFTSHGRSTSITKKCASRSECHFVGCHHSRDSEHTECRSCCEGMICNVELPTNHTNAVFAVMHAQRTSGSSAPTLYLPVLAWVFVLPLL

SEQ ID NO: 57 - protein, 35 aa, Homo sapiens
MASLGHILVFCVGLLTMAKAESPKEHDPFTYDYQS

SEQ ID NO: 58 - protein, 116 aa, Homo sapiens
MKGLRSLAATTLALFLVFVFLGNSSCAPQRLLERRNWTPQAMLYLKGAQGRRFISDQSRRKDLSDRPLPERRSPNPQLLTIPEAATILLASLQKSPEDEEKNFDQTRFLEDSLLNW

SEQ ID NO: 59 - protein, 413 aa, Homo sapiens
MKSLSLLLAVALGLATAVSAGPAVIECWFVEDASGKGLAKRPGALLLRQGPGEPPPRPDLDPELYLSVHDPAGALQAAFRRYPRGAPAPHCEMSRFVPLPASAKWASGLTPAQNCPRALDGAWLMVSISSPVLSLSSLLRPQPEPQQEPVLITMATVVLTVLTHTPAPRVRLGQDALLDLSFAYMPPTSEAASSLAPGPPPFGLEWRRQHLGKGHLLLAATPGLNGQMPAAQEGAVAFAAWDDDEPWGPWTGNGTFWLPRVQPFQEGTYLATIHLPYLQGQVTLELAVYKPPKVSLMPATLARAAPGEAPPELLCLVSHFYPSGGLEVEWELRGGPGGRSQKAEGQRWLSALRHHSDGSVSLSGHLQPPPVTTEQHGARYACRIHHPSLPASGRSAEVTLEVAGLSGPSLEDS

SEQ ID NO: 60 - protein, 147 aa, Homo sapiens
MWLWVWLIHTLHSGLQKPRERSLPEATFQNLLHPPTDLPSPCPLFESRCQVLPADTWLLEGRCSFHLTMQACFAVGRAVLSSSQLHTGITWRVQKLPASVKEHQCISTANIPNARLDSLQLPGPPGFSSFQELSDPGSSLNVGYKLT

SEQ ID NO: 61 - protein, 57 aa, Homo sapiens
MKTCWKIPVFFFVCSFLEPWASAEFEFMPRVRSLFQEQEEPEPGMEEAGEMEQKQLQ

SEQ ID NO: 62 - protein, 127 aa, Homo sapiens
MKTCWKIPVFFFVCSFLEPWASAEFKRMYDRAISAPTSPTRLSHSGKRSWEEPNWMGSPRLLNRDMKTGLSRSLQTLPTDSSTFDTGETSYLLPEPGSHPCWRAGSGQASCCGHMCRAPELPAGQVA

SEQ ID NO: 63 - protein, 118 aa, Homo sapiens [sequence omitted]

SEQ ID NO: 64 - protein, 139 aa, Homo sapiens
MKTCWKIPVFFFVCSFLEPWASAEFEFEPSQDLRLESPTRSLIMEAPRGVQVSAAAGDFKATCRKELHLQSTEGEIFLNAETIKLGNLPTGSFSSSSPSSSSSRQTVYELCVCPNGKLYLSPAGVGSTCQSSSNICLWS

SEQ ID NO: 65 - protein, 212 aa, Homo sapiens
MVMLQIPVASTRGLLAPIWATCTTSVSVRLSAPTPDIFLQLHPPLPESTDTHPPTRLFPSPPPSVSGRVSPSNRQEPKRLSTDSCPLAQESGAAHSSRPRRKSCFDSPRHRISSYFTTAPFPVVYSCTVRSTSQVQGHQLRHLSRDRTILICPEKDIPLIILNLWMDTYLLTRPSLFNENCLSFHSICEAPLDADMCLHRTSCPQPELARIH

SEQ ID NO: 66 - protein, 113 aa, Homo sapiens
MNPAAEAEFNILLATDSYKVTHYKQYPPNTSKVYSYFECREKKTENSKLRKVKYEETVFYGLQYILNKYLKGKVVTKEKIQEAKDVYKEHFQDDVFNEKGWNYILEVKDGFIF

SEQ ID NO: 67 - protein, 183 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGE

SEQ ID NO: 68 - protein, 219 aa, Homo sapiens
MKLLLWACIVCVAFARKRRFPFIGEDDNDDGHPLHPSLNIPYGIRNLPPPLYYRPVNTVPSYPGNTYTDTGLPSYPWILTSPGFPYVYHIRGFPLATQLNVPPLPPRGFPFVPPSRFFSAAAAPAAPPIAAEPAAAAPLTATPVAAEPAAGAPVAAEPAAEAPVGAEPAAEAPVAAEPAAEAPVGVEPAAEEPSPAEPATAKPAAPEPHPSPSLEQANQ

SEQ ID NO: 69 - protein, 141 aa, Homo sapiens
MTKQHELGGLLALVQNCQSEMNIKDSRAVGLSVKRLCISFVDEFCERTERPLYLAQGLFMKRETYWEVQDSGISPLLLLLSTALDCSPEAETRQSPGGRKMLQEPTLSMSLQILTGFLWVQLWNWETFLRIRTHSTDASCP

SEQ ID NO: 70 - protein, 257 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 71 - protein, 98 aa, Homo sapiens [sequence omitted]

SEQ ID NO: 72 - protein, 457 aa, Homo sapiens
MGLQTTKWPSHGAFFLKSWLIISLGLYSQVSKLLACPSVCRCDRNFVYCNERSLTSVPLGIPEGVTVLYLHNNQINNAGFPAELHNVQSVHTVYLYGNQLDEFPMNLPKNVRVLHLQENNIQTISRAALAQLLKLEELHLDDNSISTVGVEDGAFREAISLKLLFLSKNHLSSVPVGLPVDLQELRVDENRIAVISDMAFQNLTSLERLIVDGNLLTNKGIAEGTFSHLTKLKEFSIVRNSLSHPPPDLPGTHLIRLYLQDNQINHIPLTAFSNLRKLERLDISNNQLRMLTQGVFDNLSNLKQLTARNNPWFCDCSIKWVTEWLKYIPSSLNVRGFMCQGPEQVRGMAVRELNMNLLSCPTTTPGLPLFTPAPSTASPTTQPPTLSIPNPSRSYTPPTPTTSKLPTIPDWDGRERVTPPISERIQLSIHFVNDTSIQVSWLSLFTVMAYKLTWVKM

SEQ ID NO: 73 - protein, 164 aa, Homo sapiens
MPLHLAGFFFLAAYSQPCSFSRSPLQGTLPHDSGQQHLKTTADDLLGVCHQQSPGLGQKERTTQSVERTELGRLRVIDVIPQHVEGVVRTAPEVEAVKVLSEVLPPAHIQQVAGELIKALQRGVQNNEHDSQECQSLKPFQVFVSQDPIVLTGDQANLVDHKLL

SEQ ID NO: 74 - protein, 130 aa, Homo sapiens
MLPPGPLKCFFLFLFLFFFLRWSFALAAQAGVQWHDLGSPQPPPPGFKRFSCVSLLISWDCRQVPPCPANFVFLVEMGFLHVGQAGLEFLTSGDPPASASQSAGITGVSHRARPTKCLLSHNPDFLLESP

SEQ ID NO: 75 - protein, 385 aa, Homo sapiens
MFFGGEGSLTYTLVIICFLTLRLSASQNCLKKSLEDVVIDIQSSLSKGIRGNEPVYTSTQEDCINSCCSTKNISGDKACNLMIFDTRKTARQPNCYLFFCPNEEACPLKPAKGLMSYRIITDFPSLTRNLPSQELPQEDSLLHGQFSQAVTPLAHHHTDYSKPTDISWRDTLSQKFGSSDHLEKLFKMDEASAQLLAYKEKGHSQSSQFSSDQEIAHLLPENVSALPATVAVASPHTTSATPKPATLLPTNASVTPSGTSQPQLATTAPPVTTVTSQPPTTLISTVFTRAAATLQAMATTAVLTTTFQAPTDSKGSLETIPFTEISNLTLNTGNVYNPTALSMSNVESSTMNKTASWEGREASPGSSSQGSVPENQYGLPFEKWL

SEQ ID NO: 76 - protein, 189 aa, Homo sapiens
MDFWLWPLYFLPVSGALRILPEVKVEGELGGSVTIKCPLPEMHVRIYLCREMAGSGTCGTVVSTTNFIKAEYKGRVTLKQYPRKNLFLVEVTQLTESDSGVYACGAGMNTDRGKTQKVTLNVHSEYEPSWEEQPMPETPKWFHLPYLFQMPAYASSSKFVTRVTTPAQRGKVPPVHHSSPTTQITHRPR

SEQ ID NO: 77 - protein, 242 aa, Homo sapiens
MPRGFTWLRYLGIFLGVALGNEPLEMWPLTQNEECTVTGFLRDKLQYRSRLQYMKHYFPINYKISVPYEGVFRIANVTRLQRAQVSERELRYLWVLVSLSATESVQDVLLEGHPSWKYLQEVQTLLLNVQQGLTDVEVSPKVESVLSLLNAPGPNLKLVRPKALLDNCFRVMELLYCSCCKQSSVLNWQDCEVPSPQSCSPEPSLQYAATQLYPPPPWSPSSPPHSTGSVRPVRAQGEGLLP

SEQ ID NO: 78 - protein, 99 aa, Homo sapiens
MQSLMQAPLLIALGLLLAAPAQAHLKKPSQLSSFSWDNCDEGKDPAVIRSLTLEPDPIVVPGNVTPSWLTTGNYRIESVLSSSGKRLGCIKIAASLKGI

SEQ ID NO: 79 - protein, 71 aa, Homo sapiens
MELPYTNLEMAFILLAFVIFSLFTLASIYTTPDDSNEEEEHEKKGREKKRKKSEKKKNCSEEEHRIEAVEL

SEQ ID NO: 80 - protein, 205 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGT

SEQ ID NO: 81 - protein, 92 aa, Homo sapiens
MLAGAGRPGLPQGRHLCWLLCAFTLKLCQAEAPVQEEKLSASTSNLPCWLVEEFVVAEECSPCSNFRAKTTPECGPTGYVEKITCSSSKRNE

SEQ ID NO: 82 - protein, 284 aa, Homo sapiens
MRKGLRATAARCGLGLGYLLQMLVLPALALLSASGTGSAAQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRT

SEQ ID NO: 83 - protein, 185 aa, Homo sapiens
MKTCWKIPVFFFVCSFLEPWASAEFEFAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 84 - protein, 163 aa, Homo sapiens
MKTCWKIPVFFFVCSFLEPWASAEFSAIRANCHQEPSVCLQAACPESWIGFQRKCFYFSDDTKNWTSSQRFCDSQDADLAQVESFQELNFLLRYKGPSDHWIGLSREQGQPWKWINGTEWTRQLVMKEDGANLYVAKVSQVPRMNPRPVMVSYPGSRRVCLFE

SEQ ID NO: 85 - protein, 320 aa, Homo sapiens
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQC

SEQ ID NO: 86 - protein, 59 aa, Homo sapiens
MKTCWKIPVFFFVCSFLEPWASAEFEFAPLSWDLPEPRSRASKIRVHSRGNLWATGHFM

SEQ ID NO: 87 - protein, 119 aa, Homo sapiens
MKTCWKIPVFFFVCSFLEPWASAEFSSPFSFLSNVKYNFMRIIKYEFILNDALNQSIIRANDQYLTAAALHNLDEAVKFDMGAYKSSKDDAKITVILRISKTQLYVTAQDEDQPVLLKA

SEQ ID NO: 88 - protein, 133 aa, Homo sapiens [sequence omitted]

SEQ ID NO: 89 - protein, 187 aa, Homo sapiens
MYLQVETRTSSRLHLKRAPGIRSWSLLVGILSIGLAAAYYSGDSLGWKLFYVTGCLFVAVQNLEDWEEAIFDKSTGKVVLKTFSLYKKLLTLFRAGHDQVVVLLHDVRDVSVEEEKVRYFGKGYMVVLRLATGFSHPLTQSAVMGHRSDVEAIAKLITSFLELHCLESPTELSQSSDSEAGDPASQS

SEQ ID NO: 90 - protein, 191 aa, Homo sapiens
MYYLVAFQHRAWFSLFLIVRMMCSGMSHCERGPDDGAWTERVNGAAHLRTLPALLCWEQGAGQPGRGWSSSKGREEWPSGDHREEPCHDRLKKLDYANKKGKTSVTHRKQCSWPCHRRRSKGGKLRRLPVNSWLRMHVICSDLKTQTRTEVSGPTCEMHSAERHPALAAGWEAGCRMIVRGKKFCRSYQAP

SEQ ID NO: 91 - protein, 410 aa, Homo sapiens
MISAAWSIFLIGTKIGLFLQVAPLSVMAKSCPSVCRCDAGFIYCNDRFLTSIPTGIPEDATTLYLQNNQINNAGIPSDLKNLLKVERIYLYHNSLDEFPTNLPKYVKELHLQENNIRTITYDSLSKIPYLEELHLDDNSVSAVSIEEGAFRDSNYLRLLFLSRNHLSTIPWGLPRTIEELRLDDNRISTISSPSLQGLTSLKRLVLDGNLLNNHGLGDKVFFNLVNLTELSLVRNSLTAAPVNLPGTNLRKLYLQDNHINRVPPNAFSYLRQLYRLDMSNNNLSNLPQGIFDDLDNITQLILRNNPWYCGCKMKWVRDWLQSLPVKVNVRGLMCQAPEKVRGMAIKDLNAELFDCKDSGIVSTIQITTAIPNTVYPAQGQWPAPVTKQPDIKNPKLTKDHQTTGSPSRKT

SEQ ID NO: 92 - protein, 238 aa, Homo sapiens
MGTKAQVERKLLCLFILAILLCSLALGSVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGV

SEQ ID NO: 93 - protein, 109 aa, Homo sapiens
MGPHSWPVSLWPFPFFLPLCVMETRLDLAREQRTPKISRIFPSKDPGWLCFYFSGDHSRYWQFSVMALGSFKEQERGICTNLEPQSSTKLLEAQGMRISCFLFLRGFMN

SEQ ID NO: 94 - protein, 446 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRST

SEQ ID NO: 95 - protein, 61 aa, Homo sapiens
MKGLLPLAWFLACSVPAVQGGLLDLKSMIEKVTGKNALTNYGFYGCYCGWGGRGTPKDGTD

SEQ ID NO: 96 - protein, 130 aa, Homo sapiens
MESWWGLPCLAFLCFLMHARGQRDFDLADALDDPEPTKKPNSDIYPKPKPPYYPQPENPDSGGNIYPRPKPRPQPQPGNSGNSGGYFNDVDRDDGRYPPRPRPRPPAGGGGGGYSSYGNSDNTHGGDHHS

SEQ ID NO: 97 - protein, 341 aa, Homo sapiens
MVPAAGALLWVLLLNLGPRAAGAQGLTQTPTEMQRVSLRFGGPMTRSYRSTARTGLPRKTRIILEDENDAMADADRLAGPAAAELLAATVSTGFSRSSAINEEDGSSEEGVVINAGKDSTSRELPSATPNTAGSSSTRFIANSQEPEIRLTSSLPRSPGRSTEDLPGSQATLSQWSTPGSTPSRWPSPSPTAMPSPEDLRLVLMPWGPWHCHCKSGTMSRSRSGKLHGLSGRLRVGALSQLRTEHKPCTYQQCPCNRLREECPLDTSLCTDTNCASQSTTSTRTTTTPFPTIHLRSSPSLPPASPCPALAFWKRVRIGLEDIWNSLSSVFTEMQPIDRNQR

SEQ ID NO: 98 - protein, 359 aa, Homo sapiens
MKTCWKIPVFFFVCSFLEPWASAEFEFRRRFPFVRERSDSTGSSSVYFTASSGATFTDAESEGGYTTANAESDNERDSDKESEDGEDEVSCETVKMGRKDSLDLEEEAASGASSALEAGGSSGLEDVLPLLQQADELHRGDEQGKREGFQLLLNNKLVYGSRQDFLWRLARAYSDMCELTEEVSEKKSYALDGKEEAEAALEKGDESADCHLWYAVLCGQLAEHESIQRRIQSGFSFKEHVDKAIALQPENPMAHFLLGRWCYQVSHLSWLEKKTATALLESPLSATVEDALQSFLKAEELQPGFSKAGRVYISKCYRELGKNSEARWWMKLALELPDVTKEDLAIQKDLEELEVILRD

SEQ ID NO: 99 - protein, 128 aa, Homo sapiens
MSLILLCPQRLEGLLSLQAWRNIHANIPAIKLNPFSSEIPCLSPASNFIFLPQATVHLTQGKMKGLPITRTPEGACPEKSWHVTSHIHFLSSCPNLGNFALEYFQESALCFNEVFFRVPMLFFINAAF

SEQ ID NO: 100 - protein, 150 aa, Homo sapiens
  MOD_RES (29)..(30): Any amino acid
MLMISDGGCWVAAALTLTCPPQLDGGLLXXGGGSPLKMDTCPVRVSEKLQPLGRMGQGETDLGLYSGLQKPSMRSCGDSAPPSSPPPSPPVSPSPPDPSLDQARALSLSQQMPTASSLHLVFSDVLAITHLVPLSWVHDPLQCVLRELYC

SEQ ID NO: 101 - protein, 249 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTD

SEQ ID NO: 102 - protein, 183 aa, Homo sapiens
MKTCWKIPVFFFVCSFLEPWASAEFAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 103 - protein, 228 aa, Homo sapiens
MVCGSPGGMLLLRAGLLALAALCLLRVPGARAAACEPVRIPLCKSLPWNMTKMPNHLHHSTQANAILAIEQFEGLLGTHCSPDLLFFLCAMYAPICTIDFQHEPIKPCKSVCERARQGCEPILIKYRHSWPENLACEELPVYDRGVCISPEAIVTADGADFPMDSSNGNCRGASSVIRAKVKEIKTKCHDVTAVVEVKEILKSSLVNIPRDTVNLYTSSGCLCPPLYV

SEQ ID NO: 104 - protein, 253 aa, Homo sapiens
MVMSQGTYTFLTCFAGFWLIWGLIVLLCCFCSFLRRRLKRRQEERLREQNLRALELEPLELEGSLAGSPPGLAPPQPPPHRSRLEAPAHAHAHPHPHHHALPHPPPTHLSVPPRPWSYPRQAESDMSKPPCYEEAVLMAEPPPPYSEVLTDTRGLYRKIVTPFLSRRDSAEKQEQPPPSYKPLFLDRGYTSALHLPSAPRPAPPCPALCLQADRGRRVFPSWTDSELSSREPLEHGAWRLPVSIPLFGRTTAV

SEQ ID NO: 105 - protein, 144 aa, Homo sapiens
MGAVVLCRPSPLNFLIQTGTGQGLSCGSHMWRCEATPCGVCGESPVGSLLKQHRGRGKTWPVGTVSACREESEAGSLSLGWSLLPSPVGLGAVLILKRCGSLCPLPGVQGNRRGHWACFLPPDPASPTPCIIGNFHLKIFLSKV

SEQ ID NO: 106 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 107 - protein, 121 aa, Homo sapiens
MMLPQWLLLLFLLFFFLFLLTRGSLSPTKYNLLELKESCIRNQDCETGCCQRAPDNCESHCAEKGSEGSLCQTQVFFGQYRACPCLRNLTCIYSKNEKWLSIAYGRCQKIGRQKLAKKMFF

SEQ ID NO: 108 - protein, 91 aa, Homo sapiens [sequence omitted]

SEQ ID NO: 109 - protein, 229 aa, Homo sapiens
MNLEGLEMIAVLIVIVLFVKLLEQFGLIEAGLEDSVEDELEMATVRHRPEALELLEAQSKFTKKELQILYRGFKNECPSGVVNEETFKEIYSQFFPQGDSTTYAHFLFNAFDTDHNGAVSFEDFIKGLSILLRGTVQEKLNWAFNLYDINKDGYITKEEMLDIMKAIYDMMGKCTYPVLKEDAPRQHVETFFQKMDKNKDGVVTIDEFIESCQKDENIMRSMQLFENVI

SEQ ID NO: 110 - protein, 208 aa, Homo sapiens
MDPFLVLLHSVSSSLSSSELTELKFLCLGRVGKRKLERVQSGLDLFSMLLEQNDLEPGHTELLRELLASLRRHDLLRRVDDFEAGAAAGAAPGEEDLCAAFNVICDNVGKDWRRLARQLKVSDTKIDSIEDRYPRNLTERVRESLRIWKNTEKENATVAHLVGALRSCQMNLVADLVQEVQQARDLQNRSGAMSPMSWNSDASTSEAS

SEQ ID NO: 111 - protein, 109 aa, Homo sapiens
MLWWLVLLLLPTLKSVFCSLVTSLYLPNTEDLSLWLWPKPDLHSGTRTEVSTHTVPSKPGTASPCWPLAGAVPSPTVSRLEALTRAVQVAEPLGSCGFQGGPCPGRRRD

SEQ ID NO: 112 - protein, 950 aa, Homo sapiens
MTWRMGPRFTMLLAMWLVCGSEPHPHATIRGSHGGRKVPLVSPDSSRPARFLRHTGRSRGIERSTLEEPNLQPLQRRRSVPVLRLARPTEPPARSDINGAAVRPEQRPAARGSPREMIRDEGSSARSRMLRFPSGSSSPNILASFAGKNRVWVISAPHASEGYYRLMMSLLKDDVYCELAERHIQQIVLFHQAGEEGGKVRRITSEGQILEQPLDPSLIPKLMSFLKLEKGKFGMVLLKKTLQVEERYPYPVRLEAMYEVIDQGPIRRIEKIRQKGFVQKCKASGVEGQVVAEGNDGGGGAGRPSLGSEKKKEDPRRAQVPPTRESRVKVLRKLAATAPALPQPPSTPRATTLPPAPATTVTRSTSRAVTVAARPMTTTAFPTTQRPWTPSPSHRPPTTTEVITARRPSVSENLYPPSRKDQHRERPQTTRRPSKATSLESFTNAPPTTISEPSTRAAGPGRFRDNRMDRREHGHRDPNVVPGPPKPAKEKPPKKKAQDKILSNEYEEKYDLSRPTASQLEDELQVGNVPLKKAKESKKHEKLEKPEKEKKKKMKNENADKLLKSEKQMKKSEKKSKQEKEKSKKKKGGKTEQDGYQKPTNKHFTQSPKKSVADLLGSFEGKRRLLLITAPKAENNMYVQQRDEYLESFCKMATRKISVITIFGPVNNSTMKIDHFQLDNEKPMRVVDDEDLVDQRLISELRKEYGMTYNDFFMVLTDVDLRVKQYYEVPITMKSVFDLIDTFQSRIKDMEKQKKEGIVCKEDKKQSLENFLSRFRWRRRLLVISAPNDEDWAYSQQLSALSGQACNFGLRHITILKLLGVGEEVGGVLELFPINGSSVVEREDVPAHLVKDIRNYFQVSPEYFSMLLVGKDGNVKSWYPSPMWSMVIVYDLIDSMQLRRQEMAIQQSLGMRCPEDEYAGYGYHSYHQGYQDGYQDDYRHHESYHHGYPY

SEQ ID NO: 113 - protein, 68 aa, Homo sapiens
MLELLRTLGFYYILHFLGSLIHALGFKYHLSDSISNIDLSSEFQNLAILLDVSWVLQYSVSSYLTETF

SEQ ID NO: 114 - DNA, 603 bp, Homo sapiens
atgaagacctgctggaaaattccagttttcttctttgtgtgcagtttcctggaaccctgggcatctgcagaattcgtgacatttcgcatctttcaaacctgtgatgagaaaaagtttcagctacctgagaatttcacagagctctcctgctacaattatggatcaggttcagtcaagaattgttgtccattgaactgggaatattttcaatccagctgctacttcttttctactgacaccatttcctgggcgttaagtttaaagaactgctcagccatgggggctcacctggtggttatcaactcacaggaggagcaggaattcctttcctacaagaaacctaaaatgagagagttttttattggactgtcagaccaggttgtcgagggtcagtggcaatgggtggacggcacacctttgacaaagtctctgagcttctgggatgtaggggagcccaacaacatagctaccctggaggactgtgccaccatgagagactcttcaaacccaaggcaaaattggaatgatgtaacctgtttcctcaattattttcggatttgtgaaatggtaggaataaatcctttgaacaaaggaaaatctctt

SEQ ID NO: 115 - DNA, 375 bp, Homo sapiens
atggcatgccatactctcctggtttgtgccctgcttgtttctttactgccttcctgcctgccctttctgtgctggaggctcatcccattcatgctcagccctcctcgctgtctacactgtcttcttgggcaatcgtatctagtcccaagcctttttaaaaaacaagctctacccatcatggtggaattcctgtctataaaagggcacaccttcagacctccaggcacctccagagaacagtgctgtgcacaagaaaccgaattcagttgtgtgggacctgcagctgcaatcaggtttcaaactcctactgaaataactaagactgccctttgcaatcttgaagcttatataataattttaagtggtataaaataa

SEQ ID NO: 116 - DNA, 711 bp, Homo sapiens
atgaatgggctgtcgctgagtgagctctgctgcctcttctgctgcccgccctgccccggccgcatcgctgccaagctcgccttcctgccgccggaggccacctactccctggtgcctgagcccgagccggggcctggtggggccggggccgcccccttggggaccctgagagcctcctcgggcgcacccgggcgctggaagctgcacctgacggagcgtgccgacttccagtacagccagcgcgagctggacaccatcgaggtcttccccaccaagagcgcccgcggcaaccacgtctcctgcatgtatgttcgctgcgtgcctggtgccaggtacacggtcctcttctcgcacggcaatgccgtggacctgggccagatgagcagcttctacattggcctgggctcccgcctccactgcaacatcttctcctacgactactccggctacggtgccagctcgggcaggccttccgagaggaacctctatgccgacatcgacgccgcctggcaggccctgcgcaccaggtacggcatcagcccggacagcatcatcctgtacgggcagagcatcggcacggtgcccaccgtggacctggcctcgcgctacgagtgtgccgcggtggtgctgcactcgccgctcacctcgggcatgcgcgtcgccttccccgacaccaagaagacctactgcttcgacgccttccctaagtga

SEQ ID NO: 117 - DNA, 570 bp, Homo sapiens
atgcctatacttgggctcttagtctcacccctgccaccttctcagggactaatggttagcagcgatcctctaactcagtcctttatttccgattccacttgctctttcccagtcacattaaaatgtgctcaaatctccttatctttgaaagaaatactctggacccctttagctgccaccttaccgcactgtgtccattcacagccaagcctcttgaaagcattaactgccctcagtgtctacttccctactcattttccagccctctgcccccaccacagctgctttcaccaaattaccaacaacctcctggttgctgaaaatcagaggatgttcttagttcttcacttgaccaccccctttttgaaaaaaagaatttgcttgacgtctgtagtttcgcattcctggtttttcccttgcctctctggctcctccttgtcagtccctgttcaaagaggcagtgctgtctggattagtggtcagccatggtacttaagaacctgggcttgggagacaggtctgggttcatatgcggcttccacttctcagagtttcagtttcttcaactaa

SEQ ID NO: 118 - DNA, 555 bp, Homo sapiens
atgaagacctgctggaaaattccagttttcttctttgtgtgcagtttcctggaaccctgggcatctgcagaattcgaattccgcagaagtgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggcgagttccctcgggccccagctccgcctctgccaggtgtctgggctgttggccctgcggccagggtcctccctgcggatccgcaccctcccctgggcccatctcaaggctgcccccttcctcacctacttcggactcttccaggttcac

SEQ ID NO: 119 - DNA, 237 bp, Homo sapiens
atggcggcgtcggcggctctgtctgcagcggcggctgcggcggccctgtctggcctggcggtgcggctgtcgcgctcagctgcggcccgaggctcgtacggcgccttctgcaaggggctcacgcgcacgctgctcaccttcttcgacctggcctggcggctgcgcatgaacttcccctacttctacatcgtggcctcggtgatgcttaacgtccgcctgcaggtgcggatcgagtga

SEQ ID NO: 120 - DNA, 567 bp, Homo sapiens
atggctccgccctgggcaggaggagaaagaagagggccaggcaccacgtgcctgcactctccctggatgctggaggctgctccgccgtgggcaggaggagaaggaagagaactgggtgccgcctgcctgcactctctccggatgctggaggctggaggcagtgaggcagcaacagcgcgggggcgaggcgactttggagctgcctcatgcagcgacctcgccttccgctgcgcctcctcccagaacccaagaagcctggaacctgtggcgtccagccctgaaaggaggaggcggcaacccagccgcgcttttgcctgcactctccctggatgctggaggctggaggcagtgacgcagcaacagcgcgaggcgactttggagcggcctcatatagcgacctcgccttccgctgcgcgtcctcccagagcccaagaagcccggaacctgtggcatccatctctgaaaggagaagacggcaacccagccgaggcactactgggttggggtctccacgaccgagctggtctcatcaagtggcgtccaacaaggggctcaaacccgggttga

SEQ ID NO: 121 - DNA, 654 bp, Homo sapiens
atggcagtggggactcgggggactcttctgaagaagagtctcaccgtgtggttttgcggccccggggcaaggagcgccacaagagcggtgtccaccagcctccccaggcgggagcaggtgacgtggtgctgctgcagcgggagctggcccaggaggacagcctcaacaagctggcgctgcagtatggctgcaaacacattttattttagacctaatggggctggagataccaggcagaatttaattccggatttctatgcattcagagtgattaacaatggcaaagttgcagatatcaagaaagtcaacaacttcatcagagaacaagacttatatgctttgaaatctgttaagattccagtgagaaaccatgggatcctgatggagacccacaaagaactgaaaccccttctgagcccgtcttccgagaccacagtgaccgtggaactgccagaggcagacagagcaggcgcgggcaccggtgcccaggccggccaactgatgggcttctttaaggggattgaccaggatattgagcgtgcagtgcagtcagaaatctttctacatgaaagttactgcatggacacctcccatcagccactgctcccggcacctccgaagacgcctatggatggtgcagattgtggcattcag

SEQ ID NO: 122 - DNA, 213 bp, Homo sapiens
atgacttgtatggatttaagaaatgtagggaggagtttcattctaatcaaattcttcctgacacaatggttcaccctgatttaccagtttttgactacattgctgagcagagagaaaattgcagagttgacttgcagtattttgactgattcatgtcctagtgcttcggggaaattgcttaaggaagttggcgttgctcaaaagtatagttga

SEQ ID NO: 123 - DNA, 1794 bp, Homo sapiens
atgcaggcggcagtggctgtgtccgtgcccttcttgctgctctgtgtcctggggacctgccctccggcgcgctgcggccaggcaggagacgcctcattgatggagctagagaagaggaaggaaaaccgcttcgtggagcgccagagcatcgtgccactgcgcctcatctaccgctcgggcggcgaagacgaaagtcggcacgacgcgctcgacacgcgggtgcggggcgacctcggtggcccgcagttgactcatgttgaccaagcaagcttccaggttgatgcctttggaacgtcattcattctcgatgtcgtgctaaatcatgatttgctgtcctctgaatacatagagagacacattgaacatggaggcaagactgtggaagttaaaggaggagagcactgttactaccagggccatatccgaggaaaccctgactcatttgttgcattgtcaacatgccacggacttcatgggatgttctatgacgggaaccacacatatctcattgagccagaagaaaatgacactactcaagaggatttccattttcattcagtttacaaatccagactgtttgaattttccttggatgatcttccatctgaatttcagcaagtaaacattactccatcaaaatttattttgaagccaagaccaaaaaggagtaaacggcagcttcgtcgatatcctcgtaatgtagaagaagaaaccaaatacattgaactgatgattgtgaatgatcaccttatgtttaaaaaacatcggctttccgttgtacataccaatacctatgcgaaatctgtggtgaacatggcagatttaatatataaagaccaacttaagaccaggatagtattggttgctatggaaacctgggcgactgacaacaagtttgccatatctgaaaatccattgatcaccctacgtgagtttatgaaatacaggagggattttatcaaagagaaaagtgatgcagttcaccttttttcgggaagtcaatttgagagtagccggagcggggcagcttatattggtgggatttgctcgttgctgaaaggaggaggcgtgaatgaatttgggaaaactgatttaatggctgttacacttgcccagtcattagcccataatattggtattatctcagacaaaagaaagttagcaagtggtgaatgtaaatgcgaggacacgtggtccgggtgcataatgggagacactggctattatcttcctaaaaagttcacccagtgtaatattgaagagtatcatgacttcctgaatagtggaggtggtgcctgccttttcaacaaaccttctaagcttcttgatcctcctgagtgtggcaatggcttcattgaaactggagaggagtgtgattgtggaaccccggccgaatgtgtccttgaaggagcagagtgttgtaagaaatgcaccttgactcaagactctcaatgcagtgacggtctttgctgtaaaaagtgcaagtttcagcctatgggcactgtgtgccgagaagcagtaaatgattgtgatattcgtgaaacgtgctcaggaaattcaagccagtgtgcccctaatattcataaaatggatggatattcatgtgatggtgttcagggaatttgctttggaggaagatgcaaaaccagagatagacaatgcaaatacatttgggggcaaaaggtgacagcatcagacaaatattgctatgagaaactgaatattgaagggacggagaagggtaactgtgggaaagacaaagacacatggatacagtgcaacaaacgggatgtg

SEQ ID NO: 124 - DNA, 450 bp, Homo sapiens
atgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagaca

SEQ ID NO: 125 - DNA, 204 bp, Homo sapiens
atgagagaccagttgatcctcatcctttcccctgctggtattgtcagtatttttagtttagctgctctaacatgtgagtcatggtgtctcctcatagctttaatttgcatgtccctaatgactaatgatgctgagcatctttccgtgtgctcattcttttctccactttttaatcggattctttgttttcttactgttgagttt

SEQ ID NO: 126 - DNA, 1779 bp, Homo sapiens
atgcagaggctcatgatgctcctcgccacatcgggcgcctgcctgggcctgctggcagtggcagcagtggcagcagcaggtgctaaccctgcccaacgggacacccacagcctgctgcccacccaccggcgccaaaagagagattggatttggaaccagatgcacattgatgaagagaaaaacacctcacttccccatcatgtaggcaagatcaagtcaagcgtgagtcgcaagaatgccaagtacctgctcaaaggagaatatgtgggcaaggtcttccgggtcgatgcagagacaggagacgtgttcgccattgagaggctggaccgggagaatatctcagagtaccacctcactgctgtcattgtggacaaggacactggtgaaaacctggagactccttccagcttcaccatcaaagttcatgacgtgaacgacaactggcctgtgttcacgcatcggttgttcaatgcgtccgtgcctgagtcgtcggctgtggggacctcagtcatctctgtgacagcagtggatgcagacgaccccactgtgggagaccacgcctctgtcatgtaccaaatcctgaaggggaaagagtattttgccatcgataattctggacgtattatcacaataacgaaaagcttggaccgagagaagcaggccaggtatgagatcgtggtggaagcgcgagatgcccagggcctccggggggactcgggcacggccaccgtgctggtcactctgcaagacatcaatgacaacttccccttcttcacccagaccaagtacacatttgtcgtgcctgaagacacccgtgtgggcacctctgtgggctctctgtttgttgaggacccagatgagccccagaaccggatgaccaagtacagcatcttgcggggcgactaccaggacgctttcaccattgagacaaaccccgcccacaacgagggcatcatcaagcccatgaagcctctggattatgaatacatccagcaatacagcttcatcgtcgaggccacagaccccaccatcgacctccgatacatgagccctcccgcgggaaacagagcccaggtcattatcaacatcacagatgtggacgagccccccattttccagcagcctttctaccacttccagctgaaggaaaaccagaagaagcctctgattggcacagtgctggccatggaccctgatgcggctaggcatagcattggatactccatccgcaggaccagtgacaagggccagttcttccgagtcacaaaaaagggggacatttacaatgagaaagaactggacagagaagtctacccctggtataacctgactgtggaggccaaagaactggattccactggaacccccacaggaaaagaatccattgtgcaagtccacattgaagttttggatgagaatgacaatgccccggagtttgccaagccctaccagcccaaagtgtgtgagaacgctgtccatggccagctggtcctgcagatctccgcaatagacaaggacataacaccacgaaacgtgaagttcaaattcaccttgaatactgagaacaactttaccctcacggataatcacgataacacggccaacatcacagtcaagtatgggcagtttgaccgggagcataccaaggtccacttcctacccgtggtcatctcagacaatgggatgccaagtcgcacgggcaccagcacgctgaccgtggccgtgtgcaagtgcaacgagcagggcgagttcaccttctgcgaggatatggccgcccag

SEQ ID NO: 127 - DNA, 2424 bp, Homo sapiens
atgaagacctgctggaaaattccagttttcttctttgtgtgcagtttcctggaaccctgggcatctgcagaattccgacataggccttctttgctacttgaaaactaccagccatggctagacctgaaaatttcttccaaggttgatgcatctctctcagaggtggatattccatctattataaccaagaaactattaaaagcagcaatgaagcatatagaagtgatagttaaagccagacagaaagtaaaaaatacagagtttttacagcaagctgctttagaagaatatggtccagagcttcatgttgctttgagaagtcgaagagatgaattgcactatttaaggaaacttactgaactgctttttccttatattttgcctcctaaagcaacagactgcagatctctgaccttacttataagagagattctgtctggctctgtgttccttccttctttggatttcctagctgatccagatactgtgaatcatttgcttatcatcttcatagatgacagtccacctgaaaaagcaactgaaccggcttctcctttggttccattcttgcagaaatttgcagaacctagaaataaaaagccatctgtgctgaagttagaattgaagcaaatcagagagcaacaagatcttttatttcgttttatgaactttctgaaacaagaaggcgcagtgcacgtgttgcagttttgtttgactgtggaggaatttaatgatagaattttacgaccagaattatcaaatgatgaaatgctgtctcttcatgaagaattgcagaagatttataaaacatactgtttggatgaaagtattgacaaaattagatttgatcccttcattgtagaagagattcaaagaattgctgaaggcccatacatagatgttgtgaaacttcaaactatgagatgtctttttgaagcatatgaacatgttctttcccttttggagaatgtatttactcctatgttctgccatagtgatgagtatttcagacaacttttaagaggtgcagaatcaccaacacgcaattcaaaattgaacaggaacacacagaaaaggggagaatcatttggaatcagcagaataggtagcaaaattaaaggagtattcaaaagtaccacaatggagggagctatgttgcctaattatggtgtagctgaaggtgaagatgattttattgaagaaggtattgttgtaatggaagatgattctccagtggaggctgtgagcacacctaatactccccgaaaccttgctgcatggaaaattagcattccatatgtagacttttttgaggatccctcctctgaaaggaaggagaaaaaagaaagaattcctgtgttttgtattgatgttgaaagaaatgatagaagagcagttggacacgagcctgaacattggtctgtctatagaagatatcttgaattctatgtacttgaatcaaaactaacagaatttcatggtgcatttcctgatgcccagcttccttctaagaggatcattggccccaaaaattatgaattcttaaagtcaaagagggaagagttccaagaatatctacagaaacttctgcagcatccagaactgagtaatagtcaacttctggcagactttctttcccctaatggtggggaaacacaatttcttgataagatactaccagatgtaaatcttgggaaaattataaaatctgttcctggaaaactaatgaaagagaaaggtcagcatttggaaccttttatcatgaatttcattaattcttgtgagtctccaaagcctaaaccaagtagaccagaactgaccattctcagccctacttcagaaaacaacaagaagcttttcaatgatctgtttaaaaataatgcaaaccgtgctgaaaatacagagagaaagcaaaatcagaattattttatggaggtgatgactgtagaaggagtctatgattacctgatgtatgtaggacgggtagttttccaggttcctgactggcttcatcatctcttaatgggaactcgaatcctctttaaaaacaccctggaaatgtatactgattactatcttcagtgtaaactagaacagctatttcaggagcaccgtttggtctcactcataacacttctcagagatgctatattctgtgaaaacactgaacctcgctctctccaagataagcaaaaaggagcaaaacagacttttgaagaaatgatgaattacattccagatctgttagtcaagtgtattggtgaagaaaccaagtatgaaagcatcagacttctgtttgatggcttacagcaaccagtactcaacaagcagctgacttatgttttattggacattgtgatacaggaactgtttccagagctcaataaggtacaaaaggaagttacctctgtgacatcttggatg

SEQ ID NO: 128 - DNA, 1248 bp, Homo sapiens
atgaagacctgctggaaaattccagttttcttctttgtgtgcagtttcctggaaccctgggcatctgcagaattcgaattcgaagtgatttccagaggatttacactggtcagtgctgcttgcccatttaataaagctggacagcatccaagtcagcatctcatcggtcttcggaaagctgtctaccgaactctaagagccaacttccaagcagcaaggctagctaccctatatatgctgaaaaactaccccctgaactctgagagtgacaatgtaaccaactacatctgtgtggtgccttttaaagagctgggccttggacttagtgaagagcagatttcagaagaggaagcacataactttacagatggcttcagcctgcctgcattgaaggttttgttccaactctgggtggcacagagttcagagttcttcagacggttagccctattactttctacagccaattcacctcctgggcccttacttactccagcacttctgcctcatcgtatcttatctgatgtgactcaaggtctacctcatgctcattctgcctgtttggaagagcttaagcgcagctatgagttctatcggtactttgaaactcagcaccagtcagtaccgcagtgtttatccaaaactcaacagaagtcaagagaactgaataatgttcacacagcagtgcgtagcttgcagctccatctgaaagcattactgaatgaggtaataattcttgaagatgaacttgaaaagcttgtttgtactaaagaaacacaagaactagtgtcagaggcttatcccatcctagaacagaaattaaagttgattcagccccacgttcaagcaagcaacaattgctgggaagaggccatttctcagatcgacaaactgctacgaagaaatacagataaaaaaggcaagcctgaaatagcatgtgaaaacccacattgtacagtagtacctttgaagcagcctactctacacattgcagacaaagatccaatcccagaggagcaggaattagaagcttatgtagatgatatagatattgatagtgatttcagaaaggatgatttttattacttgtctcaagaagacaaagagagacagaagcgtgagcatgaagaatccaagagggtgctccaagaattaaaatctgtgctgggatttaaagcttcagaggcagaaaggcagaagtggaagcaacttctatttagtgatcatggggtaaagtccgcatggaat

SEQ ID NO: 129 - DNA, 489 bp, Homo sapiens
atggggaccaagctacagtcatggacgttctggctgtgcctcctgtgcaatggaatcaggagcaccggcctgggccggcagttggacaatggtgaccggatgagtggccagtcatggttccactcaatgctggtgaatgaactatcccctggcagtttgcaaagctcaggtccacagctgactgtggaactggaagtgccacagcacccacccctgccgccaagccgtgccccacttctgctgctaaatggagaaaggatggagcctacattagaaaccagagacctggcaaagtgggtgggacaggaggttcctggcaagcgacagacagaactacagaatgcctggctgctcctcttgaacggctggggcgagagggatactgcatgcgatggaactgataaaccagttcaaatagcattagactcactcaccgatactgtctcattgaaacacattgctcatttattcctcttcgtcatcctttga

SEQ ID NO: 130 - DNA, 1485 bp, Homo sapiens
atggagccgctggtgacctgggtggtccccctcctcttcctcttcctgctgtccaggcagggcgctgcctgcagaaccagtgagtgctgttttcaggacccgccatatccggatgcagactcaggctcggcctcgggccctagggacctgagatgctatcggatatccagtgatcgttacgagtgctcctggcagtatgagggtcccacagctggggtcagccacttcctgcggtgttgccttagctccgggcgctgctgctacttcgccgccggctcagccaccaggctgcagttctccgaccaggctggggtgtctgtgctgtacactgtcacactctgggtggaatcctgggccaggaaccagacagagaagtctcctgaggtgaccctgcagctctacaactcagttaaatatgagcctcctctgggagacatcaaggtgtccaagttggccgggcagctgcgtatggagtgggagaccccggataaccaggttggtgctgaggtgcagttccggcaccggacacccagcagcccatggaagttgggcgactgcggacctcaggatgatgatactgagtcctgcctctgccccctggagatgaatgtggcccaggaattccagctccgacgacggcagctggggagccaaggaagttcctggagcaagtggagcagccccgtgtgcgttccccctgaaaaccccccacagcctcaggtgagattctcggtggagcagctgggccaggatgggaggaggcggctgaccctgaaagagcagccaacccagctggagcttccagaaggctgtcaagggctggcgcctggcacggaggtcacttaccgactacagctccacatgctgtcctgcccgtgtaaggccaaggccaccaggaccctgcacctggggaagatgccctatctctcgggtgctgcctacaacgtggctgtcatctcctcgaaccaatttggtcctggcctgaaccagacgtggcacattcctgccgacacccacacagaaccagtggctctgaatatcagcgtcggaaccaacgggaccaccatgtattggccagcccgggctcagagcatgacgtattgcattgaatggcagcctgtgggccaggacgggggccttgccacctgcagcctgactgcgccgcaagacccggatccggctggaatggcaacctacagctggagtcgagagtctggggcaatggggcaggaaaagtgttactacattaccatctttgcctctgcgcaccccgagaagctcaccttgtggtctacggtcctgtccacctaccactttgggggcaatgcctcagcagctgggacaccgcaccacgtctcggtgaagaatcatagcttggactctgtgtctgtggactgggcaccatccctgctgagcacctgtcccggcgtcctaaaggagtatgttgtccgctgccgagatgaagacagcaaacaggtgtcagag

SEQ ID NO: 131 - DNA, 2292 bp, Homo sapiens
gccgccaccatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaacgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaagggttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccacccccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacgggatccgagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 132 - DNA, 663 bp, Homo sapiens
atgtcgggttcgtcgctgcccagcgccctggccctctcgctgttgctggtctctggctccctcctcccagggccaggcgccgctcagaacgagccaaggattgtcaccagtgaagaggtcattattcgagacagccctgttctccctgtcaccctgcagtgtaacctcacctccagctctcacacccttacatacagctactggacaaagaatggggtggaactgagtgccactcgtaagaatgccagcaacatggagtacaggatcaataagccgagagctgaggattcaggcgaataccactgcgtatatcactttgtcagcgctcctaaagcaaacgccaccattgaagtgaaagccgctcctgacatcactggccataaacggagtgagaacaagaatgaagggcaggatgccactatgtattgcaagtcagttggctacccccacccagactggatatggcgcaagaaggagaacgggatgcccatggacattgtcaatacctctggccgcttcttcatcatcaacaaggaaaattacactgagttgaacattgtgaacctgcagatcacggaagaccctggcgagtatgaatgtaatgccaccaacgccattggctccgcctctgttgtcactgtcctcagggtgcggagccacctg

SEQ ID NO: 133 - DNA, 420 bp, Homo sapiens
atgaagacctgctggaaaattccagttttcttctttgtgtgcagtttcctggaaccctgggcatctgcagaattcgaattcctccctgtgaacagccctatgaataaaggggataccgaggtgatgaaatgcatcgttgaggtcatctccgacacactttccaagcccagccccatgcctgtcagccaggaatgttttgagacactccgaggagatgaacggatcctttccattctgagacatcagaatttactgaaggagctccaagacctcgctctccaaggcgccaaggagagggcacatcagcagaagaaacacagcggttttgaagatgaactctcagaggttcttgagaaccagagcagccaggccgagctgaaagaggcggtggaagagccatcatccaaggatgttatggag

SEQ ID NO: 134 - DNA, 723 bp, Homo sapiens
atggggcccctcccagccccttcctgcacacagcgcatcacctggaaggggctcctgctcacagcatcacttttaaacttctggaacccgcccaccactgccgaagtcacgattgaagcccagccacccaaagtttctgaggggaaggatgttcttctacttgtccacaatttgccccagaatcttcctggctacttctggtacaaaggggaaatgacggacctctaccattacattatatcgtatatagttgatggtaaaataattatatatgggcctgcatacagtggaagagaaacagtatattccaacgcatccctgctgatccagaatgtcacccggaaggatgcaggaacctacaccttacacatcataaagcgaggtgatgagactagagaagaaattcgacatttcaccttcaccttatactatggtccagacctccccagaatttacccttcattcacctattaccgttcaggagaaaacctcgacttgtcctgcttcacggaatctaacccaccggcagagtatttttggacaattaatgggaagtttcagcaatcaggacaaaagctctttatcccccaaattactagaaatcatagcgggctctatgcttgctctgttcataactcagccactggcaaggaaatctccaaatccatgacagtcaaagtctctggtccctgccatggagacctgacagagtctcagtcatga

SEQ ID NO: 135 - DNA, 258 bp, Homo sapiens
atgttgcttacaggaccagcaatgcttcttcacctagagacacttctcccagcagtggcagtgccacttcagcttctttcagcactactggaatcagcctcagtgattccccctgtacccgctcagagattatccacagcagccagatggttctaccttccacaaagattgtggttgcaattctgggcttctaagttctggttacttcatatttttccttttgttcctccagccctagaggtggtagctgctttctga

SEQ ID NO: 136 - DNA, 1737 bp, Homo sapiens
atgcgcaccccacagctcgcgctcctgcaagtgttctttctggtgttccccgatggcgtccggcctcagccctcttcctccccatcaggggcagtgcccacgtctttggagctgcagcgagggacggatggcggaaccctccagtccccttcagaggcgactgcaactcgcccggccgtgcctggactccctacagtggtccctactctcgtgactccctcggcccctgggaataggactgtggacctcttcccagtcttaccgatctgtgtctgtgacttgactcctggagcctgcgatataaattgctgctgcgacagggactgctatcttctccatccgaggacagttttctccttctgccttccaggcagcgtaaggtcttcaagctgggtttgtgtagacaactctgttatcttcaggagtaattccccgtttccttcaagagttttcatggattctaatggaatcaggcagttttgtgtccatgtgaacaactcaaacttaaactatttccagaagcttcaaaaggtcaatgcaaccaacttccaggccctggctgcagagtttggaggcgaatcattcacttcaacattccaaactcaatcaccaccatctttttacagggctggggaccccattcttacttacttccccaagtggtctgtaataagcttgctgagacaacctgcaggagttggagctgggggactctgtgctgaaagcaatcctgcaggtttcctagagagtaaaagtacaacttgcactcgttttttcaagaacctggctagtagctgtaccttggattcagccctcaatgctgcctcttactataacttcacagtcttaaaggttccaagaagcatgactgatccacagaatatggagttccaggttcctgtaatacttacctcacaggctaatgctcctctgttggctggaaacacttgtcagaatgtagtttctcaggtcacctatgagatagagaccaatgggacttttggaatccagaaagtttctgtcagtttgggacaaaccaacctgactgttgagccaggcgcttccttacagcaacacttcatccttcgcttcagggcttttcaacagagcacagctgcttctctcaccagtcctagaagtgggaatcctggctatatagttgggaagccactcttggctctgactgatgatataagttactcaatgaccctcttacagagccagggtaatggaagttgctctgttaaaagacatgaagtgcagtttggagtgaatgcaatatctggatgcaagctcaggttgaagaaggcagactgcagccacttgcagcaggagatttatcagactcttcatggaaggcccagaccagagtatgttgccatctttggtaatgctgacccagcccagaaaggagggtggaccaggatcctcaacaggcactgcagcatttcagctataaactgtacttcctgctgtctcataccagtttccctggagatccaggtattgtgggcatatgtaggtctcctgtccaacccgcaagctcatgtatcaggagttcgattcctataccagtgccagtctatacaggattctcagcaagttacagaagtatctttgacaactcttgtgaactttgtggacattacccagaagccacagcctccaaggggccaacccaaaatggactggaaatggccattcgacttctttcccttcaaagtggcattcagcaga

SEQ ID NO: 137 - DNA, 396 bp, Homo sapiens
atggatctcctcgttcctgctgcgctgtctctgtgtgccatttgcttctctctacgtccctcttctctggttcctcggaactacagcttgcatgtcatggctgccctggccactctcccactctctctcgaagccttacctctcaccactaatcaactccacctccccacgatcctcaattcagattctcaagaacaagaacctgcctggcctatttcatccgttcagggaggccgattcacaggagtccatccagatgatgaggtggctgctgttggtcccagtgctcagccctcatccaatcaaccatggctggggcagggcagcatgggactaagcaaggctgcccaggcaggagaggctaccgatggggatgagagtgatgtgggcctttag

SEQ ID NO: 138 - DNA, 819 bp, Homo sapiens
atgcctcggaagcagccagctggctgcatctttctcctcacattcctgggtctgtctgggctggttggcacagttaccagaacgtactacattgggattgtggaagaatactggaactatgtaccccaagggaagaatgttattactgggaaaagtttcacagaagacaaacttgcaaccttatttctcgaaagagggcccaacaggataggcagtatttacaaaaaggctgtttacagacgcttcacggatggaacctactccatagagatccccaaacctccctggcttggattcctgggccccatcttgagggccgaagtgggtgatgtgattgtcattcatttaaagaactttgcttctcgaccttactctctgcatccacatggcgttttctacaacaaagattcagaaggagccctatacccagatggaacatctggaaggaacaaaaatgatgacatggttcctcctgggaaaaactacacctacgtctggccggtgagagaagaatatgcacctactccagccgatgccaactgcctgacctgggtgtaccattcgcacatcgacgccccaaaggacatctgctctgggctaattgggcccctgctggtctgcaaggaaggtatcctgaatagatattcagggacacggaatgatgtggatcgagagtttgttataatgtttactcttgtggatgagaatcaaagctggtacctcaatgaaaatatcaaacatttctgcaccaaccctgattcagttgacaagaaagatgctgttttccagaggagtaacaaaatgcatgaattgttctaa

SEQ ID NO: 139 - DNA, 423 bp, Homo sapiens
atgcttggaatatcggccattggcgggctttgctgcctgcacaggctctacagctgcattcctggagaaggtggaagggcagcaaaagagaaatcgcagagccagcagccagcactgaggcccagcaagctctgcaccgggggcctggggtcagccttgtgggcaaggggtgcagggaagagagcaggaaggggcacagctgctacaagcgcacgtgctgcccaagaagcacctccatacacggctctgcaggtgccgcaacgagaacagccgatgcttcccaagcatccgctacacacccaccaaggctcctggaggcgtgaagtcccacaaggcaaggcccccagtcctaggagggcaagtgggcctggactcctgtggctccccactgccatcatatctatctacagggcacagtcctga

SEQ ID NO: 140 - DNA, 696 bp, Homo sapiens
atgaagacctgctggaaaattccagttttcttctttgtgtgcagtttcctggaaccctgggcatctgcagaattcgaattcttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtat

SEQ ID NO: 141 - DNA, 285 bp, Homo sapiens
atgaagacctgctggaaaattccagttttcttctttgtgtgcagtttcctggaaccctgggcatctgcagaattcgaattcgcaccatcaattatagctgctcaacgtcaaacgatattagcagaatacaatatgtcttgtgatgatacaggaaaactaattttgaaacctaggcctcatgttcaatgccaatcttcactaattgctattggacgtaaaacagcccttcttcgaataagtgatacagcaaaaagccataaaggattccttttgcagttggatatg

SEQ ID NO: 142 - DNA, 1080 bp, Homo sapiens
atgagtgaaggggcggccgctgcctcgccacctggtgccgcttcggcagccgccgcctcggccgaggagggcaccgcggcggctgcggcggcggcagcggcgggcgggggcccggacggcggcggcgaaggggcggccgagcccccccgggagttacgctgtagcgactgcatcgtgtggaaccggcagcagacgtggctgtgcgtggtacctctgttcatcggcttcatcggcctggggctcagcctcatgcttctcaaatggatcgtggtgggctccgtcaaggagtacgtgcccaccgacctagtggactccaaggggatgggccaggaccccttcttcctctccaagcccagctctttccccaaggccatggagaccaccaccactaccacttccaccacgtcccccgccaccccctccgccgggggtgccgcctcctccaggacgcccaaccggattagcactcgcctgaccaccatcacgcgggcgcccactcgcttccccgggcaccgggtgcccatccgggccagcccgcgctccaccacagcacggaacactgcggcccctgcgacggtcccgtccaccacggccccgttcttcagtagcagcacgctgggctcccgacccccggtgccaggaactccaagtacccaggcaatgccctcctggcctactgcggcatacgctacctcctcctaccttcacgattctactccctcctggaccctgtctccctttcaggatgctgcctcctcttcttcctcttcttcctcctccgctaccaccaccacaccagaaactagcaccagccccaaatttcatacgacgacatattccacagagcgatccgagcacttcaaaccctgccgagacaaggaccttgcatactgtctcaatgatggcgagtgctttgtgatcgaaaccctgaccggatcccataaacactgtcggtgcaaagaaggctaccaaggagtccgttgtgatcaatttctgccgaaaactgattccatcttatcggatccaacagaccacttggggattgaattcatggagagtgaagaagtttatcaaaggcag

SEQ ID NO: 143 - DNA, 1068 bp, Homo sapiens
atgaagacctgctggaaaattccagttttcttctttgtgtgcagtttcctggaaccctgggcatctgcagaattcgaattcccttacctccgagttaccgtcatgcccttgctgactttgccacaaaccagacaaagagccaaaaatatttatgacatcttgccttggcgacaggaagacctggggagacatgagtcgaggagtatgcgcattttcagtactgagagcctcctctccagaaattctgagagcccggagcatgtgccctcccaagcaggcaatgccttccaggagcatacagcccacatccatgccacagagtacgcggtgggtatctatgacaacgccatggtcccccagatgtgtgggaacctcactccctcggcacactgcatcaatgtcagagcttccagagactgcgcaagcatttcttcagaggattcgcatgattatgtcaatgtccccacagcagaagagattgctgagactctagcttctaccaaaagcccttccagaaatctctttgttcttcccagtacccagaagctggagtttactgaggaaagagatgagggctgtggagatgctggtgactgcaccagtttgtattctccaggagctgaggacagtgattcactcagcaatggagaaggttcttctcagatctcaaatgactatgtcaacatgacagggttggatctcagtgccatccaggaaaggcagctctgggtggcttttcagtgctgcagagactatgaaaatgttccagcagcagatcccagtggaagccagcagcaggctgagaaagatgtgccatcctcaaacataggtcatgtcgaggacaagacagatgatcccgggacccatgtccaatgtgtcaaaaggacattccttgcttcaggggattatgcagactttcagccattcacacagagtgaggacagtcagatgaaacatagagaagagatgtcaaatgaggactccagtgactatgaaaatgtgctaactgccaagttaggaggcagggactctgagcaggggcctggcactcagctccttcctgatgaa

SEQ ID NO: 144 - DNA, 552 bp, Homo sapiens
atggtcaggatctggacaacgataatgatagtattaatcctcctattaagaatcggcccaaacaaaccctcgctgtcagggcgacaggcgcccgcccaagcccagacctcggacctggttccaagcctgttcccgctgggtctctgggcgcccggtttctgcacctggagctcgcccgatgaggacaagaggccgaggaagccggttccggggacgggcaacagggactcagggaccagaaggcggctgcaggacgcgaccgagcaggaccccaggcccgggaacgacgtcgcgagcgctgagactgccgggcctcccagcccatctggcattcgagcgcaggaccgggcgccccggcaccgccgcgcgccacccgctaggatgccggtggccccagcgccctcagccgacggagagccgctgcaggaacagggaggaggccttttccaccgcacccggagcgtttactacgggctggagctgaatacctggatgaaagtggagaggctgttcgtggagaagttccatcagtcgttttccttggacaattaa

SEQ ID NO: 145 - DNA, 1725 bp, Homo sapiens
atgactgccgagggacccagcccgcctgcccgttggcacagacgcctccccgggctctgggcggcggcgctgctcctgctcggtctgccgcgcctttcggtgcgggcggatggaaagttctttgtgctggagtctcagaatggctctcagggcctacaactggaggctgctcggctttcctgcaagagcaggggcgctcacctggcatctgcagacgagctgcggagagtggtacaggattgctcctttgcggtgtgcaccactggctggctagcagatggtactcttggaacaactgtgtgtagcaaaggaagtggagaacagcaaatcatgagagctgtcgatgtgagaattgagagcaacccagttcctggtggcacatacagtgccctttgtattaaggatgaagagaagccgtgtggagacccgccttcgttcccacacaccatcctgcagggccgcaccggcttggaaatgggggatgaactgctgtacgtgtgtgccccaggccacatcatgggccaccgggagaccgccttcaccttgctatgtaacagctgtggggagtggtacggcctggtgcaggcctgtgggaaagatgaggctgaggcacacattgactatgaagataacttccctgatgacagatctgtgtcattcagagagctcatggaggattcccggacagaggcagatgaggacaggggtcagggagactcctctgaggaggctccaaaacaggaccgtctggtctccatttctgtggggagagaaaacatagcccgggataaagtctttgtgccaaccacaggcttgcctggtgctgggagcagtgtccccgcagattcaccaggatcacggctgctccagaagcacttgttctggtttcctgctgaggctttccacaagcctgggttggaaaaggaggtggatgatgacaccaaaaagcagttttctgctggagacaaccacagtggtgtaaaattggtcccaggtgaacctgaaaccaaggtgatctacggcaacactgatggtccctcggggccatttgtgggcaagaatgacagcaaggcaggagatccagtggtgagcagcagtgatgagtcctggttagatggctaccctgtgacagagggggcttggaggaagacagaggcagaagaggaagaagatggggacagaggggatgggtcagtagggctggatgaaaacgtcctagttactcctgatcagcccattcttgtggaagttaagaagcccaagagtagcaccctcacaccaagcgagggcatgacccatagttcagttcttccatctcaaatgctagatgtggaagctttggcgctcagacccgtgaatgcttccgagactgagggcattggggatggtgacttgacgaagtaccagtcaactctaccctggagattcatcacagaggaatctcccatggccaccctgtcctatgagctcaccagctccaccctggagatattaacagtgaacactgtcaagcagacacctaaccacatcccctcaacgatcatggcaaccacccagcctccagtagaaaccactgttcctgagatccaggatagcttcccatacctgctgtctgaagacttctttggacaggaaggccccgggccaggtgcaagtgaggagcttcatcccaccttggagtcgtgtgtgggggacggatgtcctggcctcagcagaggccctgtg

SEQ ID NO: 146 - DNA, 240 bp, Homo sapiens
atggctcggggctcgctgcgccggttgctgcggctcctcgtgctggggctctggctggcgttgctgcgctccgtggccggggagcaagcgccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggccc

SEQ ID NO: 147 - DNA, 1209 bp, Homo sapiens
atggcccggggctcggcgctcccgcggcggccgctgctgtgcatcccggccgtctgggcggccgccgcgcttctgctctcagtgtcccggacttcaggtgaagtggaggttctggatccgaacgaccctttaggaccccttgatgggcaggacggcccgattccaactctgaaaggttactttctgaattttctggagccagtaaacaatatcaccattgtccaaggccagacggcaattctgcactgcaaggtggcaggaaacccaccccctaacgtgcggtggctaaagaatgatgccccggtggtgcaggagccgcggcggatcatcatccggaagacagaatatggttcacgactgcgaatccaggacctggacacgacagacactggctactaccagtgcgtggccaccaacgggatgaagaccattaccgccactggcgtcctgtttgtgcggctgggtccaacgcacagcccaaatcataactttcaggatgattaccacgaggatgggttctgccagccttaccggggaattgcctgtgcacgcttcattggcaaccggaccatttatgtggactcgcttcagatgcagggggagattgaaaaccgaatcacagcggccttcaccatgatcggcacgtctacgcacctgtcggaccagtgctcacagttcgccatcccatccttctgccacttcgtgtttcctctgtgcgacgcgcgctcccgggcacccaagccgcgtgagctgtgccgcgacgagtgcgaggtgctggagagcgacctgtgccgccaggagtacaccatcgcccgctccaacccgctcatcctcatgcggcttcagctgcccaagtgtgaggcgctgcccatgcctgagagccccgacgctgccaactgcatgcgcattggcatcccagccgagaggctgggccgctaccatcagtgctataacggctcaggcatggattacagaggaacggcaagcaccaccaagtcaggccaccagtgccagccgtgggccctgcagcacccccacagccaccacctgtccagcacagacttccctgagcttggaggggggcacgcctactgccggaaccccggaggccagatggagggcccctggtgctttacgcagaataaaaacgtacgcatggaactgtgtgacgtaccctcgtgtagtccccgagacagcagcaagatgggg

SEQ ID NO: 148 - DNA, 510 bp, Homo sapiens
atgctccgggccgcgctgcccgcgctcctgctgccgttgctgggcctcgccgctgctgccgtcgcggactgtccttcatctacttggattcagttccaagacagttgttacatttttctccaagaagccatcaaagtagaaagcatagaggatgtcagaaatcagtgtactgaccatggagcggacatgataagcatacataatgaagaagaaaatgcttttatactggatactttgaaaaagcaatggaaaggcccagatgatatcctactaggcatgttttatgacacagatgatgcgagtttcaagtggtttgataattcaaatatgacatttgataagtggacagaccaagatgatgatgaggatttagttgacacctgtgcttttctgcacatcaagacaggtgaatggaaaaaaggaaattgtgaagtttcttctgtggaaggaacactatgcaaaacagctatcccatacaaaaggaaatatttatcagataaccacatttta

SEQ ID NO: 149 - DNA, 2238 bp, Homo sapiens
atggcccgtgcccaggcgctcgtgctggcactcaccttccagctctgcgcgccggagaccgagactccggcagctggctgcaccttcgaggaggcaagtgacccagcagtgccctgcgagtacagccaggcccagtacgatgacttccagtgggagcaagtgcgaatccaccctggcacccgggcacctgcggacctgccccacggctcctacttgatggtcaacacttcccagcatgccccaggccagcgagcccatgtcatcttccagagcctgagcgagaatgatacccactgtgtgcagttcagctacttcctgtacagccgggacgggcacagcccgggcaccctgggcgtctacgtgcgcgttaatgggggccccctgggcagtgctgtgtggaatatgactggatcccacggccgtcagtggcaccaggctgagctggctgtcagcactttctggcccaatgaatatcaggtgctgtttgaggccctcatctccccagaccgcaggggctacatgggcctagatgacatcctgcttctcagctacccctgcgcaaaggccccacacttctcccgcctgggcgacgtggaggtcaacgcgggccagaacgcgtcgttccagtgcatggccgcgggcagagcggccgaggccgaacgcttcctcttgcaacggcagagcggggcgctggtgccggcggcgggcgtgcggcacatcagccaccggcgcttcctggccactttcccgctggctgccgtgagccgcgccgagcaggacctgtaccgctgtgtgtcccaggccccgcgcggcgcgggcgtctctaacttcgcggagctcatcgtcaaggagcccccaactcccatcgcgcccccacagctgctgcgtgctggccccacctacctcatcatccagctcaacaccaactccatcattggcgacgggccgatcgtgcgcaaggagattgagtaccgcatggcgcgcgggccctgggctgaggtgcacgccgtcagcctgcagacctacaagctgtggcacctcgaccccgacacagagtatgagatcagcgtgctctcacgtcccggagacggcggcactggccgccctgggccacccctcatcagccgcaccaaatgcgcagagcccatgagggcccccaaaggcttggcttttgctgagatccaggcccgtcagctggccctgcagtgggaaccactgggctacaacgtgacgcgttgccacacctatactgtgtcgctgtgctatcactacaccctgggcagcagccacaaccagaccatccgagagtgtgtgaagacagagcaaggtgtcagccgctacaccatcaagaacctgctgccctatcggaacgttcacgtgaggcttgtcctcactaaccctgaggggcgcaaagagggcaaggaggtcactttccagacggatgaggatgtgcccagtgggattgcagccgagtccctgaccttcactccactggaggacatgatcttcctcaagtgggaggagccccaggagcccaatggtctcatcacccagtatgagatcagctaccagagcatcgagtcatcagacccggcagtgaacgtgccaggcccacgacgtaccatctccaagctccgcaatgagacctaccatgtcttctccaacctgcacccaggcaccacctacctgttctccgtgcgggcccgcacaggcaaaggcttcggccaggcggcactcactgagataaccactaacatctctgctcccagctttgattatgccgacatgccgtcacccctgggcgagtctgagaacaccatcaccgtgctgctgaggccggcacagggccgcggtgcgcccatcagtgtgtaccaggtgattgtggaggaggagcgggcgcggaggctgcggcgggagccaggtggacaggactgcttcccagtgccattgaccttcgaggcggcgctggcccgaggcctggtgcactacttcggggccgaactggcggccagcagtctacctgaggccatgccctttaccgtgggtgacaaccagacctaccgaggcttctggaacccaccacttgagcctaggaaggcctatctcatctacttccaggcagcaagccacctgaagggggagacccggctgaattgcatccgcattgccaggaaagctgcctgcaaggaaagcaagcggcccctggaggtgtcccagagatcggaggagatggggctt

SEQ ID NO: 150 - DNA, 315 bp, Homo sapiens
atgccgtttccagtgagagtcgaggtggacatggtgcgagtgatggaggtgttcctggcacagttgcggttgctctttgggattgctcagccccagctgcctccaaaatgcctgctttcagggcctacgagtgaagggctaatgacctgggagctagaccggctgctctgggctcggtcagtggagaacctggccacagccaccaccacccttacctccctggcgcagcttctgggcaagatcagcaacattgtcattaaggacgacgtggcatctgaggtgtacaaggctgtagctgccgtccagaagtcggca

SEQ ID NO: 151 - DNA, 177 bp, Homo sapiens
atgaattggaaggttcttgagcacgtgcccctgctgctgtatatcttggcagcaaaaacattaattctctgcctgacatttgctggggtgaaaatgtatcaaagaaaaaggttggaggcaaaacaacaaaaactggaggctgaaaggaagaagcaatcagagaaaaaagataactga

SEQ ID NO: 152 - DNA, 708 bp, Homo sapiens
atgaaatatgtcttctatttgggtgtcctcgctgggacatttttctttgctgactcatctgttcagaaagaagaccctgctccctatttggtgtacctcaagtctcacttcaacccctgtgtgggcgtcctcatcaaacccagctgggtgctggccccagctcactgctatttaccaaatctgaaagtgatgctgggaaatttcaagagcagagtcagagacggtactgaacagacaattaaccccattcagatcgtccgctactggaactacagtcatagcgccccacaggatgacctcatgctcatcaagctggctaagcctgccatgctcaatcccaaagtccagccccttaccctcgccaccaccaatgtcaggccaggcactgtctgtctactctcaggtttggactggagccaagaaaacagtggccgacaccctgacttgcggcagaacctggaggcccccgtgatgtctgatcgagaatgccaaaaaacagaacaaggaaaaagccacaggaattccttatgtgtgaaatttgtgaaagtattcagccgaatttttggggaggtggccgttgctactgtcatctgcaaagacaagctccagggaatcgaggtggggcacttcatgggaggggacgtcggcatctacaccaatgtttacaaatatgtatcctggattgagaacactgctaaggacaagtga

SEQ ID NO: 153 - DNA, 267 bp, Homo sapiens
atgaagacctgctggaaaattccagttttcttctttgtgtgcagtttcctggaaccctgggcatctgcagaattcaaaaggaacaaaaggagaattatgaggatattcagtgtgccacctacagaggaaactttgtcagagcccaacttttatgacacgataagcaagattcgtttaagacaacaactggaaatgtattccatttcaagaaagtacgactatcagcagccacaaaaccaagctgacagtgtgcaactctcattggaa

SEQ ID NO: 154 - DNA, 570 bp, Homo sapiens
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctga

SEQ ID NO: 155 - DNA, 1917 bp, Homo sapiens
atgggagaccacctggaccttctcctaggagtggtgctcatggccggtcctgtgtttggaattccttcctgctcctttgatggccgaatagccttttatcgtttctgcaacctcacccaggtcccccaggtcctcaacaccactgagaggctcctgctgagcttcaactatatcaggacagtcactgcttcatccttcccctttctggaacagctgcagctgctggagctcgggagccagtatacccccttgactattgacaaggaggccttcagaaacctgcccaaccttagaatcttggacctgggaagtagtaagatatacttcttgcatccagatgcttttcagggactgttccatctgtttgaacttagactgtatttctgtggtctctctgatgctgtattgaaagatggttatttcagaaatttaaaggctttaactcgcttggatctatccaaaaatcagattcgtagcctttaccttcatccttcatttgggaagttgaattccttaaagtccatagatttttcctccaaccaaatattccttgtatgtgaacatgagctcgagcccctacaagggaaaacgctctccttttttagcctcgcagctaatagcttgtatagcagagtctcagtggactggggaaaatgtatgaacccattcagaaacatggtgctggagatagtagatgtttctggaaatggctggacagtggacatcacaggaaactttagcaatgccatcagcaaaagccaggccttctctttgattcttgcccaccacatcatgggtgccgggtttggcttccataacatcaaagatcctgaccagaacacatttgctggcctggccagaagttcagtgagacacctggacctttcacatgggtttgtcttctccctgaactcacgagtctttgagacactcaaggatttgaaggttctgaaccttgcctacaacaagataaataagattgcagatgaagcattttacggacttgacaacctccaagttctcaatttgtcatataaccttctgggggaactttgcagttcgaatttctatggactacctaaggtagcctacattgatttgcaaaagaatcacattgcaataattcaagaccaaacattcaaattcctggaaaaattacagaccttggatctccgagacaatgctcttacaaccattcattttattccaagcatacccgatatcttcttgagtggcaataaactagtgactttgccaaagatcaaccttacagcgaacctcatccacttatcagaaaacaggctagaaaatctagatattctctactttctcctacgggtacctcatctccagattctcattttaaatcaaaatcgcttctcctcctgtagtggagatcaaaccccttcagagaatcccagcttagaacagcttttccttggagaaaatatgttgcaacttgcctgggaaactgagctctgttgggatgtttttgagggactttctcatcttcaagttctgtatttgaatcataactatcttaattcccttccaccaggagtatttagccatctgactgcattaaggggactaagcctcaactccaacaggctgacagttctttctcacaatgatttacctgctaatttagagatcctggacatatccaggaaccagctcctagctcctaatcctgatgtatttgtatcacttagtgtcttggatataactcataacaagttcatttgtgaatgtgaacttagcacttttatcaattggcttaatcacaccaatgtcactatagctgggcctcctgcagacatatattgtgtgtaccctgactcgctctctggggtttccctcttctctctttccacggaaggttgtgatgaagaggaagtcttaaagtccctaaag

SEQ ID NO: 156 - DNA, 417 bp, Homo sapiens
atggcggcttccaggtgggcgcgcaaggccgtggtcctgctttgtgcctctgacctgctgctgctgctgctactgctaccaccgcctgggtcctgcgcggccgaaggctcgcccgggacgcccgacgagtctaccccacctccccggaagaagaagaaggatattcgcgattacaatgatgcagacatggcgcgtcttctggagcaatgggagacccctgagccccttccagtgctgcctgaggtgccctctacctgtgcctgcctctcgtctgccagtttgatttggacctgtttttcccacctcagcccccatgctcttgtcaaacgtgtttggcctccagccaaacagggcctgggaggaaaagagagtcctgcttctgcctggcttccccatcggggaggggagttgaagtga

SEQ ID NO: 157 - DNA, 1152 bp, Homo sapiens
ttcgactgctcagggaagtacagatgtcgctcatcctttaagtgtatcgagctgatagctcgatgtgacggagtctcggattgcaaagacggggaggacgagtaccgctgtgtccgggtgggtggtcagaatgccgtgctccaggtgttcacagctgcttcgtggaagaccatgtgctccgatgactggaagggtcactacgcaaatgttgcctgtgcccaactgggtttcccaagctatgtgagttcagataacctcagagtgagctcgctggaggggcagttccgggaggagtttgtgtccatcgatcacctcttgccagatgacaaggtgactgcattacaccactcagtatatgtgagggagggatgtgcctctggccacgtggttaccttgcagtgcacagcctgtggtcatagaaggggctacagctcacgcatcgtgggtggaaacatgtccttgctctcgcagtggccctggcaggccagccttcagttccagggctaccacctgtgcgggggctctgtcatcacgcccctgtggatcatcactgctgcacactgtgtttatgacttgtacctccccaagtcatggaccatccaggtgggtctagtttccctgttggacaatccagccccatcccacttggtggagaagattgtctaccacagcaagtacaagccaaagaggctgggcaatgacatcgcccttatgaagctggccgggccactcacgttcaatgaaatgatccagcctgtgtgcctgcccaactctgaagagaacttccccgatggaaaagtgtgctggacgtcaggatggggggccacagaggatggagcaggtgacgcctcccctgtcctgaaccacgcggccgtccctttgatttccaacaagatctgcaaccacagggacgtgtacggtggcatcatctccccctccatgctctgcgcgggctacctgacgggtggcgtggacagctgccagggggacagcggggggcccctggtgtgtcaagagaggaggctgtggaagttagtgggagcgaccagctttggcatcggctgcgcagaggtgaacaagcctggggtgtacacccgtgtcacctccttcctggactggatccacgagcagatggagagagacctaaaaacc

SEQ ID NO: 158 - DNA, 390 bp, Homo sapiens
atgagatcactgttaatactgtcatgggactcttggagtattgcttttttggctggaaacctctgtggccagtggcacctttgcccaagttttgcttgggcatccaggagccggcataggtgtctgctccctgcaagactgcagctggaccaggtgtactgtaagcaggcagcttccacagctggcactggggaacatggtggtggccagaagcttggagacaccaggaactgcagagctccaaagagggtgtcacaggcctgtatcaggaatctcctaggtctgggctccctgaagggccacagctcttccctccttctctcttctctccttcttgtcacccgcaatgtggcaagcaaggggtgtgtttcagccctgtttgtgttatag

SEQ ID NO: 159 - DNA, 387 bp, Homo sapiens
atgttgcttgctcctattttggcaatgctaaccattctttcctgcttaaaatgcttcatgacactgtgctttctaagtgtccctttctcctctttggctggttcttttcagtctccttcaggggctgtacttcctttacctgatccctctcaattcttcttcagagttccaaatcctggctccccgttactctcagtaaataccatccctctggttgaacttaacctacgtattcgttctgtttttgttacacaaatatttaatgagcggctattacgtgacgggttctttttaataagcactggggataaaaacatgcatgactgctgcattagtggtctgattggaaagaatgcaaaatgtgcatggaatcatgaaagcatataa

SEQ ID NO: 160 - DNA, 315 bp, Homo sapiens
atgtcatcccttcaaagagaccttcttggaccacccccgccattctctatcctatttctctcttttattttctttgaagcacttacccactttcagaaactatctggcttgctcatgtgtttgtctctagattgtctacttcccctcctagactgtactctccacaagagagcaaagctgtgtctgttgcattcactgttgtcttcccagaagcaagcagcatgctggagaccctcaaatacatgtgaaatgaagggacaccttgtgttatttcacctgtttccacctggttcccaagtggcaagcttgcattga

SEQ ID NO: 161 - DNA, 207 bp, Homo sapiens
atgacctgtggtttaacccttttgatcactaccaccattatcagcaccagactgagcagctatatccttttattaatcatggtcattcattcattcattcattcacaaaatatttatgatgtatttactctgcaccaggtcccatgccaagcactggggacacagttatggcaaagtagacaaagcatttgttcatttggagcttag

SEQ ID NO: 162 - DNA, 231 bp, Homo sapiens
atgtcgcgggtatcgctgcgctgctgttgtggcttctggtgctggcgccggcgtggtgggggtagggtgggtgccgcgctcagatgcgggggtccctacctccccggcccaggccgctcccattgctggggaacttgcagctgcagtccggaggcctggaccgcgcactccattcccgacaggaatgggggaacggaattctggattcgtgaaggtggggctggggactag

SEQ ID NO: 163 - DNA, 582 bp, Homo sapiens
atgtcgggttcgtcgctgcccagcgccctggccctctcgctgttgctggtctctggctccctcctcccagggccaggcgccgctcagaacgccgctcctgacatcactggccataaacggagtgagaacaagaatgaagggcaggatgccactatgtattgcaagtcagttggctacccccacccagactggatatggcgcaagaaggagaacgggatgcccatggacattgtcaatacctctggccgcttcttcatcatcaacaaggaaaattacactgagctgaacattgtgaacctgcagatcacggaagaccctggcgagtatgaatgtaatgccaccaacgccattggctccgcctctgttgtcactgtcctcagggtgcggagccacctggccccactctggcctttcttgggaattctggctgaaattatcatccttgtggtgatcattgttgtgtatgagaagaggaagaggccagatgaggttcctgacgatgatgaaccagctggaccaatgaaaaccaactctaccaacaatcacaaagataaaaacttgcgccagagaaacacaaattaa

SEQ ID NO: 164 - DNA, 468 bp, Homo sapiens
atgtggcaagacccagaagtatgggtgaccttatgtcagaggaagtggctttcttattgcagcaaaaattcccgtaccaatgtcagtgctaggattgtgaagatggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccggtctactgatcactgccttccctgctctgccatcatacaaattatctcataggattgttttgaggattaaacaaaacaccaaccacgggtcacagattatgacattttcaaggctcgttggcaaaagggacttgccttgtctcagacgagactatggattttga

SEQ ID NO: 165 - DNA, 2565 bp, Homo sapiens
atggttatttctttgaactcatgcctgagctttatttgtttattgttatgccactggattgggacagcatcacctctgaatcttgaagaccctaatgtgtgtagccactgggaaagctactcagtgactgtgcaagagtcatacccacatccctttgatcaaatttactacacgagctgcactgacattctaaactggtttaaatgcacgcggcacagagtcagctatcggacagcctatcgacatggggagaagactatgtataggcgcaagtctcagtgttgtcctggattttatgaaagcggggaaatgtgtgtcccccactgtgctgataaatgtgtccatggtcgctgtattgctccaaacacctgtcagtgtgagcctggctggggagggaccaactgctccagtgcctgcgatggtgatcactggggtccccactgcaccagccggtgccagtgcaaaaatggggctctgtgcaaccccatcaccggggcttgccactgtgctgcgggcttccggggctggcgctgcgaggaccgctgtgagcagggcacctatggtaacgactgtcatcagagatgccagtgccagaatggagccacctgcgaccacgtcacgggggaatgccgctgcccaccaggatacaccggagccttctgtgaggatctttgtcctcctggtaaacatggtccacagtgtgagcagagatgcccttgtcaaaatggaggagtgtgtcatcacgtcactggagaatgctcttgcccttctggctggatgggcacagtgtgtggtcagccttgccccgagggtcgctttggaaagaactgttcccaagaatgccagtgccataatggagggacgtgtgatgctgccacaggccaatgtcattgcagtccaggatacacaggggaacggtgccaggatgagtgtcctgttgggacctatggcgttctctgtgctgagacctgccagtgtgtcaacggagggaagtgttaccacgtgagcggcgcatgcctctgtgaagcaggctttgctggcgagcgctgcgaagcacgcctgtgtcctgaggggctctacggcatcaaatgtgacaaacggtgtccctgccacttggaaaacactcatagctgtcaccccatgtctggagagtgtgcctgcaagccgggctggtcaggactctactgtaatgagacatgttctcctggattctacggggaagcttgccagcagatctgcagctgccaaaatggggcagactgtgacagtgtgactggaaagtgcacctgtgccccaggattcaaaggaattgactgctctaccccatgccctctgggaacctatgggataaactgttcctctcgctgtggctgtaaaaatgatgcagtctgctctcctgtggacgggtcttgtacttgcaaggcaggctggcacggggtggactgctccatcagatgtcccagtggcacatggggctttggctgtaacttaacatgccagtgcctcaacgggggagcctgcaacaccctggacgggacctgcacgtgtgcacctggatggcgcggggagaaatgcgaacttccctgccaggatggcacgtacgggctgaactgtgctgagcgctgcggctgcagccacgcagatggctgccaccctaccacgggccattgccgctgcctccccggatggtcaggtgtccactgtgacagcgtgtgtgctgagggacgctggggccccaactgctccctgccctgctactgtaaaaatggggcttcatgctcccctgacgatggcatctgcgagtgtgcaccaggcttccgaggcaccacttgtcagaggatctgctcccctggtttttatgggcatcgctgcagccagacatgcccacagtgcgttcacagcagcgggccctgccaccacatcaccggcctgtgtgactgcttgcctggcttcacaggcgccctctgcaatgaagtgtgtcccagtggcagatttgggaaaaactgtgcaggaatttgtacctgcaccaacaacggaacctgtaaccccattgacagatcttgtcagtgttaccccggttggattggcagtgactgctctcaaccatgtccacctgcccactggggcccaaactgcatccacacgtgcaactgccataatggagctttctgcagcgcctacgatggggaatgtaaatgcactcctggctggacagggctctactgcactcagagatgtcctctagggttttatggaaaagattgtgcactgatatgccaatgtcaaaacggagctgactgcgaccacatttctgggcagtgtacttgccgcactggattcatgggacggcactgtgagcagaagtgcccttcaggaacatatggctatggctgtcgccagatatgtgattgtctgaacaactccacctgcgaccacatcactgggacctgttactgcagccccggatggaagggagcgagatgtgatcaagctggtgttatcatagttggaaatctgaacagcttaagccgaaccagtactgctctccctgctgattcctaccag

SEQ ID NO: 166 - DNA, 648 bp, Homo sapiens
atggacgtggggcccagctccctgccccaccttgggctgaagctgctgctgctcctgctgctgctgcccctcaggggccaagccaacacaggctgctacgggatcccagggatgcccggcctgcccggggcaccagggaaggatgggtacgacggactgccggggcccaagggggagccaggaatcccagccattcccgggatccgaggacccatgggcatccctggagagccaggtgaggagggcagatacaagcagaaattccagtcagtgttcacggtcactcggcagacccaccagccccctgcacccaacagcctgatcagattcaacgcggtcctcaccaacccgcagggagattatgacacgagcactggcaagttcacctgcaaagtccccggcctctactactttgtctaccacgcgtcgcatacagccaacctgtgcgtgctgctgtaccgcagcggcgtcaaagtggtcaccttctgtggccacacgtccaaaaccaatcaggtcaactcgggcggtgtgctgctgaggttgcaggtgggcgaggaggtgtggctggctgtcaatgactactacgacatggtgggcatccagggctctgacagcgtcttctccggcttcctgctcttccccgactag

SEQ ID NO: 167 - DNA, 567 bp, Homo sapiens
atgaagacctgctggaaaattccagttttcttctttgtgtgcagtttcctggaaccctgggcatctgcagaattcgaattctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtg

SEQ ID NO: 168 - DNA, 426 bp, Homo sapiens
atgaagacctgctggaaaattccagttttcttctttgtgtgcagtttcctggaaccctgggcatctgcagaattccgaagtgaccccaacttcaagaacaggcttcgagaacgaagaaagaaacagaagcttgccaaggagagagctgggctttccaagttacctgaccttaaagatgctgaagctgttcagaagttcttccttgaagaaatacagcttggtgaagagttactagctcaaggtgaatatgagaagggcgtagaccatctgacaaatgcaattgctgtgtgtggacagccacagcagttactgcaggtcttacagcaaactcttccaccaccagtgttccagatgcttctgactaagctcccaacaattagtcagagaattgtaagtgctcagagcttggctgaagatgatgtggaa

SEQ ID NO: 169 - DNA, 552 bp, Homo sapiens
atgttatataagagttcggaccgcccagcacacaaggtcagcatgctgctcctctgtcacgctctcgctatagctgttgtccagatcgttatcttctcagaaagctgggcatttgccaagaacatcaacttctataatgtgaggcctcctctcgaccctacaccatttccaaatagcttcaagtgctttacttgtgaaaacgcaggggataattataactgcaatcgatgggcagaagacaaatggtgtccacaaaatacacagtactgtttgacagttcatcacttcaccagccacggaagaagcacatccatcaccaaaaagtgtgcctccagaagtgaatgtcattttgtcggttgccaccacagccgagattctgaacatacggagtgtaggtcttgctgtgaaggaatgatctgcaatgtagaattacccaccaatcacactaatgcagtgtttgccgtaatgcacgctcagagaacatctggcagcagtgcccccacactctacctaccagtgcttgcctgggtctttgtgcttccattgctgtga

SEQ ID NO: 170 - DNA, 105 bp, Homo sapiens
atggcgtctcttggccacatcttggttttctgtgtgggtctcctcaccatggccaaggcagaaagtccaaaggaacacgacccgttcacttacgactaccagtcc

SEQ ID NO: 171 - DNA, 351 bp, Homo sapiens
atgaagggactcagaagtctggcagcaacaaccttggctcttttcctggtgtttgttttcctgggaaactccagctgcgctccgcagagactgttggagagaaggaactggactcctcaagctatgctctacctgaaaggggcacagggtcgccgcttcatctccgaccagagccggagaaaggacctctccgaccggccactgccggaaagacgaagcccaaatccccaactactaactattccggaggcagcaaccatcttactggcgtcccttcagaaatcaccagaagatgaagaaaaaaactttgatcaaaccagattcctggaagacagtctgcttaactggtga

SEQ ID NO: 172 - DNA, 1239 bp, Homo sapiens
atgaagtccctgtctctgctcctcgctgtggctttgggcctggcgaccgccgtctcagcaggacccgcggtgatcgagtgttggttcgtggaggatgcgagcggaaagggcctggccaagagacccggtgcactgctgttgcgccagggaccgggggaaccgccgccccggccggacctcgaccctgagctctatctcagtgtacacgaccccgcgggcgccctccaggctgccttcaggcggtatccccggggcgcccccgcaccacactgcgagatgagccgcttcgtgcctctccccgcctctgcgaaatgggccagcggcctgacccccgcgcagaactgcccgcgggccctggatggggcttggctgatggtcagcatatccagcccagtcctcagcctctccagcctcttgcgaccacagccagagcctcagcaggagcctgttctcatcaccatggcaacagtggtactgactgtcctcacccacacccctgcccctcgagtgagactgggacaagatgctctgctggacttgagctttgcctacatgccccccacctccgaggccgcctcatctctggctccgggtccccctccctttgggctagagtggcgacgccagcacctgggtaagggacatctgctcctggctgcaactcctgggctgaatggccagatgccagcagcccaagaaggggccgtggcatttgctgcttgggatgatgatgagccatggggcccatggaccggaaatgggaccttctggctgcctagagttcaaccctttcaggagggcacctatctggccaccatacacctgccatacctgcaaggacaggtcaccctggagcttgctgtgtacaaaccccccaaagtgtccctgatgccagcaacccttgcacgggccgccccaggggaggcacccccggaattgctctgccttgtgtcccacttctacccttctgggggcctggaggtggagtgggaactccggggtggcccagggggccgctctcagaaggccgaggggcagaggtggctctcggccctgcgccaccattccgatggctctgtcagcctctctgggcacttgcagccgcccccagtcaccactgagcagcatggggcacgctatgcctgtcgaattcaccatcccagcctgcctgcctcggggcgcagcgctgaggtcaccctggaggtagcaggtctttcagggccctcccttgaggacagc

SEQ ID NO: 173 - DNA, 444 bp, Homo sapiens
atgtggctttgggtatggctaatccacaccctgcactctggccttcagaagcccagggagagaagcctcccagaagcaaccttccagaaccttctccatcctcccaccgacctccccagcccctgtcctctctttgaatctcgttgtcaagttcttcccgcagacacctggctccttgaggggaggtgcagctttcatctgaccatgcaggcgtgctttgcagtgggtagagcagtgctttcctcctcccagctgcacactggaatcacctggagagtccagaaacttcccgcctcagtgaaagaacatcagtgtatcagcacagcaaatattccaaatgccaggctggattccctccaattaccaggacctccaggcttctcctcctttcaagaactttctgaccctggatccagtctaaatgttggttataaactcacctga

SEQ ID NO: 174 - DNA, 171 bp, Homo sapiens
atgaagacctgctggaaaattccagttttcttctttgtgtgcagtttcctggaaccctgggcatctgcagaattcgaattcatgccccgagtccggagcttgttccaggagcaggaagagcccgagcctggcatggaggaggctggtgagatggagcagaagcagctgcag

SEQ ID NO: 175 - DNA, 381 bp, Homo sapiens
atgaagacctgctggaaaattccagttttcttctttgtgtgcagtttcctggaaccctgggcatctgcagaattcaagcggatgtacgatcgggcgatcagtgcccctaccagccccacccgcctgagccattcggggaaaaggagctgggaagaacccaactggatgggctccccacgactgctgaacagggacatgaagacgggcctgagccggtccttgcagacccttcccacagactcctccaccttcgacacaggagaaacttcttacttactaccggaaccgggcagccatccctgctggagagcaggctcgggccaagcaagctgctgtggacatatgtgccgagctccggagcttcctgcgggccaagttgcc

SEQ ID NO: 176 - DNA, 357 bp, Homo sapiens
atgggggacagcctggcagggatgccggtgtttgcagccgtggctgggtggctgcagctgtgcccaggaggatggggctcccatcccacaaactcagaaggcttaggtggtgatcccacaatctccgtggagcatgcagccctggtggcaccttccctgctgcagctgctatcttcacagctaccgctacagacaggctgctgctgcaatctgtggaaatttcagaaacaggaaaatcacctgctctctcaaaaaacaggatgctcatctgtcattttcaagtgttttaaatttttgattcagaagaataaagctacattttcaaatgaaatgtgcaagccaatttcatccttttga

SEQ ID NO: 177 - DNA, 417 bp, Homo sapiens
atgaagacctgctggaaaattccagttttcttctttgtgtgcagtttcctggaaccctgggcatctgcagaattcgaattcgagccatcccaagatctcaggcttgaatcacccaccagatccttgatcatggaagctccccgtggggtccaggtgagtgctgctgcaggagacttcaaggccacctgcaggaaggagctccatctgcaatctacagaaggggagatatttttaaatgcagagacaatcaagctgggaaatctaccaactggctccttctcatcttcttcacccagctcctcaagttctcgacagacagtgtatgaactctgcgtctgccccaatggcaaactttacctttctccagcaggagtaggttccacttgtcagtccagtagcaacatctgcctgtggagc

SEQ ID NO: 178 - DNA, 639 bp, Homo sapiens
atggtcatgctgcaaattcccgtggccagcacccgaggcctgctggctccgatctgggccacgtgtacaaccagcgtctccgtgcgcctgtccgctccaacacctgacattttccttcaactccaccctcccctccccgagtccacagacacacacccacccacacgcctcttcccttcccctccaccctcagtcagcggcagagtcagtccatccaataggcaggagcccaaaagactgtccacggacagctgtcctctcgcccaggagagtggggctgcacacagcagcaggcccaggaggaaatcgtgctttgatagccccaggcatcgtatttcatcgtatttcacaactgctccatttcctgtggtctattcttgcacagttaggtctacaagccaggttcaaggccaccaactgaggcacctgtcccgagacaggaccattctaatctgccctgaaaaggacatcccattgatcatactaaatttgtggatggatacctatctgttgaccagaccctccctcttcaatgaaaactgcctctccttccactccatctgtgaggcacccttggatgctgacatgtgcttacataggacttcctgtccacagccagaattagccagaattcactga

SEQ ID NO: 179 - DNA, 342 bp, Homo sapiens
atgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgaggtaaaagatggtttcatcttttga

SEQ ID NO: 180 - DNA, 549 bp, Homo sapiens
atgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaa

SEQ ID NO: 181 - DNA, 660 bp, Homo sapiens
atgaagcttctcctttgggcctgcattgtatgtgttgcttttgcaaggaagagacggttccccttcattggtgaggatgacaatgacgatggtcacccacttcatccatctctgaatattccttatggcatacggaatttaccacctcctctttattatcgcccagtgaatacagtccccagttaccctgggaatacttacactgacacagggttaccttcgtatccctggattctaacttctcctggattcccctatgtctatcacatccgtggttttcccttagctactcagttgaatgttcctcctctccctcctaggggtttcccgtttgtccctccttcaaggtttttttcagcagctgcagcacccgctgccccacctattgcagctgagcctgctgcagctgcacctcttacagccacacctgtagcagctgagcctgctgcaggggcccctgttgcagctgagcctgctgcagaggcacctgttggagctgagcctgctgcagaggcacctgttgcagctgagcctgctgcagaggcacctgttggagtggagccagctgcagaggaaccttcaccagctgagcctgctacagccaagcctgctgccccagaacctcacccttctccctctcttgaacaggcaaatcagtga

SEQ ID NO: 182 - DNA, 426 bp, Homo sapiens
atgacgaagcaacatgaattagggggcctcttggccttggtccaaaattgtcaatcagagatgaacataaaggactccagagcagtgggactgtctgtcaaaagactctgtatatcttttgtggatgagttttgtgagagaacagagagaccattgtacctggcacaagggctcttcatgaaaagggagacttactgggaggtgcaagacagtggcatttctcctctcctcttgctgctcagcacagccctggattgcagccccgaggctgagaccagacaaagcccgggaggcagaaagatgctccaagaaccaacactatcaatgtctttgcaaatcctcacaggattcctgtgggtccagctttggaactgggaaacctttcttcggatccgcactcattccactgatgccagctgcccctga

SEQ ID NO: 183 - DNA, 771 bp, Homo sapiens
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgac

SEQ ID NO: 184 - DNA, 297 bp, Homo sapiens
atgaattttattttacttgtcacacctgtcttaataaactggagttttgctgctaaagaactcttctctctgggggcagagcttctatttatggcacatagacatcagctaggcttttgggaatcgtttgtgttctttgtggaaatgtcctttagaagcacccatgaagtagtgtgttcagactgtgcacacagaaaacaggctctgccttcacatgtgagacggtggacttttcctctggacaaaatgacagcatcctggcgactccacagtggagctgagcgccactccctgtag

SEQ ID NO: 185 - DNA, 1371 bp, Homo sapiens
atgggcctacagaccacaaagtggcccagccatggggcttttttcctgaagtcttggcttatcatttccctggggctctactcacaggtgtccaaactcctggcctgccctagtgtgtgccgctgcgacaggaactttgtctactgtaatgagcgaagcttgacctcagtgcctcttgggatcccggagggcgtaaccgtactctacctccacaacaaccaaattaataatgctggatttcctgcagaactgcacaatgtacagtcggtgcacacggtctacctgtatggcaaccaactggacgaattccccatgaaccttcccaagaatgtcagagttctccatttgcaggaaaacaatattcagaccatttcacgggctgctcttgcccagctcttgaagcttgaagagctgcacctggatgacaactccatatccacagtgggggtggaagacggggccttccgggaggctattagcctcaaattgttgtttttgtctaagaatcacctgagcagtgtgcctgttgggcttcctgtggacttgcaagagctgagagtggatgaaaatcgaattgctgtcatatccgacatggccttccagaatctcacgagcttggagcgtcttattgtggacgggaacctcctgaccaacaagggtatcgccgagggcaccttcagccatctcaccaagctcaaggaattttcaattgtacgtaattcgctgtcccaccctcctcccgatctcccaggtacgcatctgatcaggctctatttgcaggacaaccagataaaccacattcctttgacagccttctcaaatctgcgtaagctggaacggctggatatatccaacaaccaactgcggatgctgactcaaggggtttttgataatctctccaacctgaagcagctcactgctcggaataacccttggttttgtgactgcagtattaaatgggtcacagaatggctcaaatatatcccttcatctctcaacgtgcggggtttcatgtgccaaggtcctgaacaagtccgggggatggccgtcagggaattaaatatgaatcttttgtcctgtcccaccacgacccccggcctgcctctcttcaccccagccccaagtacagcttctccgaccactcagcctcccaccctctctattccaaaccctagcagaagctacacgcctccaactcctaccacatcgaaacttcccacgattcctgactgggatggcagagaaagagtgaccccacctatttctgaacggatccagctctctatccattttgtgaatgatacttccattcaagtcagctggctctctctcttcaccgtgatggcatacaaactcacatgggtgaaaatg

SEQ ID NO: 186 - DNA, 495 bp, Homo sapiens
atgccactgcacctggcaggcttcttctttttagctgcctattcacagccctgcagcttctccaggagtcctctgcagggcaccctcccccatgactctgggcagcagcatttgaaaacgactgcagatgatctccttggtgtctgccaccagcagtctccagggctaggccagaaggaaaggaccacacagagtgtagaaaggactgagttggggcgcctacgggttatcgatgtcatcccgcagcacgttgagggtgtagtacgcacggccccggaagtagaggcggtgaaggtgctcagtgaggtccttcctccagctcacatacagcaggttgcaggtgaactgatcaaagctcttcagcgtggagttcagaacaatgagcatgacagccaggaatgtcagagtcttaaacccttccaagtctttgtttcccaggaccccatagtactgactggggatcaggccaacctggtagatcacaaattgctctga

SEQ ID NO: 187 - DNA, 393 bp, Homo sapiens
atgttgccacctgggccactaaaatgcttttttctttttctttttctttttttttttttgagatggagtttcgctcttgctgcccaggctggagtgcaatggcatgatctcggctcaccgcaacctccgcctcccgggttcaagcgattctcctgtgtcagcctcctgattagctgggattgcaggcaggtaccaccatgccccgctaattttgtatttttagtagagatggggtttctccatgttggtcaggctggtcttgaattcctgacctcaggtgatccgcctgcctcagcctcccagagtgctgggattacaggtgtgagccaccgtgcccggccaactaaatgccttctttcccataacccagactttctgcttgaatccccatga

SEQ ID NO: 188 - DNA, 1155 bp, Homo sapiens
atgttcttcgggggagaagggagcttgacttacactttggtaataatttgcttcctgacactaaggctgtctgctagtcagaattgcctcaaaaagagtctagaagatgttgtcattgacatccagtcatctctttctaagggaatcagaggcaatgagcccgtatatacttcaactcaagaagactgcattaattcttgctgttcaacaaaaaacatatcaggggacaaagcatgtaacttgatgatcttcgacactcgaaaaacagctagacaacccaactgctacctatttttctgtcccaacgaggaagcctgtccattgaaaccagcaaaaggacttatgagttacaggataattacagattttccatctttgaccagaaatttgccaagccaagagttaccccaggaagattctctcttacatggccaattttcacaagcagtcactcccctagcccatcatcacacagattattcaaagcccaccgatatctcatggagagacacactttctcagaagtttggatcctcagatcacttggagaaactatttaagatggatgaagcaagtgcccagctccttgcttataaggaaaaaggccattctcagagttcacaattttcctctgatcaagaaatagctcatctgctgcctgaaaatgtgagtgcgctcccagctacggtggcagttgcttctccacataccacctcggctactccaaagcccgccacccttctacccaccaatgcttcagtgacaccttctgggacttcccagccacagctggccaccacagctccacctgtaaccactgtcacttctcagcctcccacgaccctcatttctacagtttttacacgggctgcggctacactccaagcaatggctacaacagcagttctgactaccacctttcaggcacctacggactcgaaaggcagcttagaaaccataccgtttacagaaatctccaacctaactttgaacacagggaatgtgtataaccctactgcactttctatgtcaaatgtggagtcttccactatgaataaaactgcttcctgggaaggtagggaggccagtccaggcagttcctcccagggcagtgttccagaaaatcagtacggccttccatttgaaaaatggctt

SEQ ID NO: 189 - DNA, 567 bp, Homo sapiens
atggacttctggctttggccactttacttcctgccagtatcgggggccctgaggatcctcccagaagtaaaggtagagggggagctgggcggatcagttaccatcaagtgcccacttcctgaaatgcatgtgaggatatatctgtgccgggagatggctggatctggaacatgtggtaccgtggtatccaccaccaacttcatcaaggcagaatacaagggccgagttactctgaagcaatacccacgcaagaatctgttcctagtggaggtaacacagctgacagaaagtgacagcggagtctatgcctgcggagcgggcatgaacacagaccggggaaagacccagaaagtcaccctgaatgtccacagtgaatacgagccatcatgggaagagcagccaatgcctgagactccaaaatggtttcatctgccctatttgttccagatgcctgcatatgccagttcttccaaattcgtaaccagagttaccacaccagctcaaaggggcaaggtccctccagttcaccactcctcccccaccacccaaatcacccaccgccctcga

SEQ ID NO: 190 - DNA, 729 bp, Homo sapiens
atgccccggggcttcacctggctgcgctatcttgggatcttccttggcgtggccttggggaatgagcctttggagatgtggcccttgacgcagaatgaggagtgcactgtcacgggttttctgcgggacaagctgcagtacaggagccgacttcagtacatgaaacactacttccccatcaactacaagatcagtgtgccttacgagggggtgttcagaatcgccaacgtcaccaggctgcagagggcccaggtgagcgagcgggagctgcggtatctgtgggtcttggtgagcctcagtgccactgagtcggtgcaggacgtgctgctcgagggccacccatcctggaagtacctgcaggaggtgcagacgctgctgctgaatgtccagcagggcctcacggatgtggaggtcagccccaaggtggaatccgtgttgtccctcttgaatgccccagggccaaacctgaagctggtgcggcccaaagccctgctggacaactgcttccgggtcatggagctgctgtactgctcctgctgtaaacaaagctccgtcctaaactggcaggactgtgaggtgccaagtcctcagtcttgcagcccagagccctcattgcagtatgcggccacccagctgtaccctccgcccccgtggtcccccagctccccgcctcactccacgggctcggtgaggccggtcagggcacagggcgagggcctcttgccctga

SEQ ID NO: 191 - DNA, 300 bp, Homo sapiens
atgcagtccctgatgcaggctcccctcctgatcgccctgggcttgcttctcgcggcccctgcgcaagcccacctgaaaaagccatcccagctcagtagcttttcctgggataactgtgatgaagggaaggaccctgcggtgatcagaagcctgactctggagcctgaccccatcgtcgttcctggaaatgtgacccccagttggctcaccaccgggaactaccgcatagagagcgtcctgagcagcagtgggaagcgtctgggctgcatcaagatcgctgcctctctaaagggcatataa

SEQ ID NO: 192 - DNA, 216 bp, Homo sapiens
atggagcttccctacaccaacttggaaatggcattcattttattggcttttgttatcttttccttatttaccctggcttccatctacactactccggatgacagtaatgaagaggaagaacatgaaaaaaagggaagggaaaagaaaaggaaaaagtctgaaaagaagaaaaattgctcagaggaagagcacagaattgaagctgttgagctatga

SEQ ID NO: 193 - DNA, 615 bp, Homo sapiens
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcact

SEQ ID NO: 194 - DNA, 276 bp, Homo sapiens
atgcttgcgggtgccgggaggcctggcctcccccagggccgccacctctgctggttgctctgtgctttcaccttaaagctctgccaagcagaggctcccgtgcaggaagagaagctgtcagcaagcacctcaaatttgccatgctggctggtggaagagtttgtggtagcagaagagtgctctccatgctctaatttccgggctaaaactacccctgagtgtggtcccacaggatatgtagagaaaatcacatgcagctcatctaagagaaatgag

SEQ ID NO: 195 - DNA, 852 bp, Homo sapiens
atgaggaaaggtctgcgggcgacagcggcccgctgcggactgggactgggatacttgctgcaaatgctcgtgctacctgccctggccctgctcagcgccagcggcactggctccgccgcccaagatgatgacttttttcatgaactcccagaaacttttccttctgatccacctgagcctctgccacatttccttattgagcctgaagaagcttatattgtgaagaataagcctgtgaacctgtactgtaaagcaagccctgccacccagatctatttcaagtgtaatagtgaatgggttcatcagaaggaccacatagtagatgaaagagtagatgaaacttccggtctcattgtccgggaagtgagcattgagatttcgcgccagcaagtggaagaactctttggacctgaagattactggtgccagtgtgtggcctggagctccgcgggtaccacaaagagccggaaggcgtatgtgcgcattgcatatctacggaagacatttgagcaggaacccctaggaaaggaagtgtctttggaacaggaagtcttactccagtgtcgaccacctgaagggatcccagtggctgaggtggaatggttgaaaaatgaagacataattgatcccgttgaagatcggaatttttatattactattgatcacaacctcatcataaagcaggcccgactctctgatactgcaaattacacctgtgttgccaaaaacattgttgccaagaggaaaagtacaactgccactgtcatagtctatgtcaacggtggctggtccacctggacggagtggtctgtgtgtaacagccgctgtggacgagggtatcagaaacgtaca

SEQ ID NO: 196 - DNA, 555 bp, Homo sapiens
atgaagacctgctggaaaattccagttttcttctttgtgtgcagtttcctggaaccctgggcatctgcagaattcgaattcgcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctg

SEQ ID NO: 197 - DNA, 489 bp, Homo sapiens
atgaagacctgctggaaaattccagttttcttctttgtgtgcagtttcctggaaccctgggcatctgcagaattcagcgcaataagagctaactgccatcaagagccatcagtatgtcttcaagctgcatgcccagaaagctggattggttttcaaagaaagtgtttctatttttctgatgacaccaagaactggacatcaagtcagaggttttgtgactcacaagatgctgatcttgctcaggttgaaagcttccaggaactgaatttcctgttgagatataaaggcccatctgatcactggattgggctgagcagagaacaaggccaaccatggaaatggataaatggtactgaatggacaagacagttagtcatgaaagaagatggtgccaacttgtatgttgcaaaggtttcacaagttcctcgaatgaatccaagacctgtcatggtttcctatcctgggagcaggagagtgtgcctatttgaa

SEQ ID NO: 198 - DNA, 960 bp, Homo sapiens
atgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactcaaccagtgcccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgc

SEQ ID NO: 199 - DNA, 177 bp, Homo sapiens
atgaagacctgctggaaaattccagttttcttctttgtgtgcagtttcctggaaccctgggcatctgcagaattcgaattcgccccgctcagctgggatctcccggagccccgcagccgagccagcaagatccgagtgcactcgcgaggcaacctctgggccaccggtcacttcatg

SEQ ID NO: 200 - DNA, 354 bp, Homo sapiens
atgaagacctgctggaaaattccagttttcttctttgtgtgcagtttcctggaaccctgggcatctgcagaattctcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggca

SEQ ID NO: 201 - DNA, 402 bp, Homo sapiens
atgaaggacttacttcactcactgctgggaatgctgccaagtcagctctcgggtatcagccttcctgatggattgtcttggctaaggaaaactgcttcccccaaaatcacacttcctctcaggagcggcccatatccagtaaccgagcaccattgcaatatgacttgggacaactcaaaagagccatcccagatccagtgctcccaaggagttgctgacacttggttggactacagtgcagctcctcctctccttttgccccaccctgcgtcatcaccaccttccaaagtagatccaagagcactctctaatttgcctcacatgctaatttctgtctcggagaaacagcttcctgagaattcaacctggaactgtgaccatggcctatatggagctacataa

SEQ ID NO: 202 - DNA, 564 bp, Homo sapiens
atgtacctgcaggtggagacccgcaccagctcccgcctccatctgaagagggctccaggcatccggtcctggtccctgctggttggaatcttgtcgattggcctggctgctgcctactacagcggagatagcctgggctggaagctcttctacgtcacaggctgcctgtttgtggctgtgcagaacttggaggactgggaggaagccatcttcgacaagagcacagggaaggttgttttgaagacgttcagcctctacaagaagctgctgactcttttcagagctggccacgaccaggtggtggtcctgctccatgatgtccgtgatgtgagcgtggaggaggagaaggtccggtacttcgggaaaggctacatggtggtgctccggcttgcgacgggcttctcccaccccctcacgcagagtgcagtcatgggccaccgcagtgatgtggaagccatcgccaagctcatcaccagcttcctggagctgcactgccttgagagccccacagagctgtctcagagcagcgacagtgaggccggtgaccctgcaagccagagctga

SEQ ID NO: 203 - DNA, 576 bp, Homo sapiens
atgtactatttagttgcttttcagcatagagcttggttttccctttttttaattgtaagaatgatgtgctctggcatgtcacactgtgaaaggggaccagatgatggagcctggactgaaagggtgaatggggccgctcacctcagaactctccctgctttgctttgctgggagcagggagcagggcagcctgggagaggctggagttcctcaaagggcagagaagaatggccttcaggggaccacagggaggaaccatgccatgatagactcaaaaagctagattatgctaataaaaaggggaagacatctgtgacacacaggaaacagtgttcgtggccttgccatagaaggcgcagtaaaggaggaaaactccggagactccctgtgaattcttggctaagaatgcacgttatctgcagtgatctaaaaacacaaacgagaacagaagtgagtggccctacctgtgagatgcacagtgctgagcggcacccagcgctggctgcaggatgggaggctggctgccgtatgattgttcgtgggaagaaattttgtagaagttatcaagctccttaa

SEQ ID NO: 204 - DNA, 1230 bp, Homo sapiens
atgatcagcgcagcctggagcatcttcctcatcgggactaaaattgggctgttccttcaagtagcacctctatcagttatggctaaatcctgtccatctgtgtgtcgctgcgatgcgggtttcatttactgtaatgatcgctttctgacatccattccaacaggaataccagaggatgctacaactctctaccttcagaacaaccaaataaataatgctgggattccttcagatttgaaaaacttgctgaaagtagaaagaatatacctataccacaacagtttagatgaatttcctaccaacctcccaaagtatgtaaaagagttacatttgcaagaaaataacataaggactatcacttatgattcactttcaaaaattccctatctggaagaattacatttagatgacaactctgtctctgcagttagcatagaagagggagcattccgagacagcaactatctccgactgcttttcctgtcccgtaatcaccttagcacaattccctggggtttgcccaggactatagaagaactacgcttggatgataatcgcatatccactatttcatcaccatctcttcaaggtctcactagtctaaaacgcctggttctagatggaaacctgttgaacaatcatggtttaggtgacaaagttttcttcaacctagttaatttgacagagctgtccctggtgcggaattccctgactgctgcaccagtaaaccttccaggcacaaacctgaggaagctttatcttcaagataaccacatcaatcgggtgcccccaaatgctttttcttatctaaggcagctctatcgactggatatgtccaataataacctaagtaatttacctcagggtatctttgatgatttggacaatataacacaactgattcttcgcaacaatccctggtattgcgggtgcaagatgaaatgggtacgtgactggttacaatcactacctgtgaaggtcaacgtgcgtgggctcatgtgccaagccccagaaaaggttcgtgggatggctattaaggatctcaatgcagaactgtttgattgtaaggacagtgggattgtaagcaccattcagataaccactgcaatacccaacacagtgtatcctgcccaaggacagtggccagctccagtgaccaaacagccagatattaagaaccccaagctcactaaggatcaccaaaccacagggagtccctcaagaaaaaca

SEQ ID NO: 205 - DNA, 714 bp, Homo sapiens
atggggacaaaggcgcaagtcgagaggaaactgttgtgcctcttcatattggcgatcctgttgtgctccctggcattgggcagtgttacagtgcactcttctgaacctgaagtcagaattcctgagaataatcctgtgaagttgtcctgtgcctactcgggcttttcttctccccgtgtggagtggaagtttgaccaaggagacaccaccagactcgtttgctataataacaagatcacagcttcctatgaggaccgggtgaccttcttgccaactggtatcaccttcaagtccgtgacacgggaagacactgggacatacacttgtatggtctctgaggaaggcggcaacagctatggggaggtcaaggtcaagctcatcgtgcttgtgcctccatccaagcctacagttaacatcccctcctctgccaccattgggaaccgggcagtgctgacatgctcagaacaagatggttccccaccttctgaatacacctggttcaaagatgggatagtgatgcctacgaatcccaaaagcacccgtgccttcagcaactcttcctatgtcctgaatcccacaacaggagagctggtctttgatcccctgtcagcctctgatactggagaatacagctgtgaggcacggaatgggtatgggacacccatgacttcaaatgctgtgcgcatggaagctgtggagcggaatgtgggggtc

SEQ ID NO: 206 - DNA, 330 bp, Homo sapiens
atggggcctcacagttggccagtgtccctgtggcctttccccttcttccttccactctgtgtcatggagacccgacttgatttggcaagagaacaacgtacacctaagatttcccgaatatttcccagcaaggaccctggctggctatgtttctacttcagtggtgaccacagcagatactggcagttttctgttatggctctggggtcttttaaggagcaggaacgtggaatatgtacaaacctggagccacagtcaagtactaagctccttgaagcacaaggcatgagaatatcatgttttcttttccttagaggatttatgaattaa

SEQ ID NO: 207 - DNA, 1338 bp, Homo sapiens
atgaccgcgccgggcgccgccgggcgctgccctcccacgacatggctgggctccctgctgttgttggtctgtctcctggcgagcaggagtatcaccgaggaggtgtcggagtactgtagccacatgattgggagtggacacctgcagtctctgcagcggctgattgacagtcagatggagacctcgtgccaaattacatttgagtttgtagaccaggaacagttgaaagatccagtgtgctaccttaagaaggcatttctcctggtacaagacataatggaggacaccatgcgcttcagagataacacccccaatgccatcgccattgtgcagctgcaggaactctctttgaggctgaagagctgcttcaccaaggattatgaagagcatgacaaggcctgcgtccgaactttctatgagacacctctccagttgctggagaaggtcaagaatgtctttaatgaaacaaagaatctccttgacaaggactggaatattttcagcaagaactgcaacaacagctttgctgaatgctccagccaagatgtggtgaccaagcctgattgcaactgcctgtaccccaaagccatccctagcagtgacccggcctctgtctcccctcatcagcccctcgccccctccatggcccctgtggctggcttgacctgggaggactctgagggaactgagggcagctccctcttgcctggtgagcagcccctgcacacagtggatccaggcagtgccaagcagcggccacccaggagcacctgccagagctttgagccgccagagaccccagttgtcaaggacagcaccatcggtggctcaccacagcctcgcccctctgtcggggccttcaaccccgggatggaggatattcttgactctgcaatgggcactaattgggtcccagaagaagcctctggagaggccagtgagattcccgtaccccaagggacagagctttccccctccaggccaggagggggcagcatgcagacagagcccgccagacccagcaacttcctctcagcatcttctccactccctgcatcagcaaagggccaacagccggcagatgtaactggtacagccttgcccagggtgggccccgtgaggcccactggccaggactggaatcacaccccccagaagacagaccatccatctgccctgctcagagaccccccggagccaggctctcccaggatctcatcactgcgcccccagggcctcagcaacccctccaccctctctgctcagccacagctttccagaagccactcctcgggcagcgtgctgccccttggggagctggagggcaggaggagcacc

SEQ ID NO: 208 - DNA, 186 bp, Homo sapiens
atgaaaggcctcctcccactggcttggttcctggcttgtagtgtgcctgctgtgcaaggaggcttgctggacctaaaatcaatgatcgagaaggtgacagggaagaacgccctgacaaactacggcttctacggctgttactgcggctggggcggccgaggaacccccaaggatggcaccgattga

SEQ ID NO: 209 - DNA, 390 bp, Homo sapiens
atggagagctggtggggacttccctgtcttgcgttcctgtgttttctaatgcacgcccgaggtcaaagagactttgatttggcagatgcccttgatgaccctgaacccaccaagaagccaaactcagatatctacccaaagccaaaaccaccttactacccacagcccgagaatcccgacagcggtggaaatatctacccaaggccaaagccacgccctcaaccccagcctggcaattccggcaacagtggaggttacttcaatgatgtggaccgtgatgacggacgctacccgcccaggcccaggccacggccgcctgcaggaggtggcggcggtggctactccagttatggcaactccgacaacacgcacggtggagatcaccattca

SEQ ID NO: 210 - DNA, 1026 bp, Homo sapiens
atggtccccgccgccggcgcgctgctgtgggtcctgctgctgaatctgggtccccgggcggcgggggcccaaggcctgacccagactccgaccgaaatgcagcgggtcagtttacgctttgggggccccatgacccgcagctaccggagcaccgcccggactggtcttccccggaagacaaggataatcctagaggacgagaatgatgccatggccgacgccgaccgcctggctggaccagcggctgccgagctcttggccgccacggtgtccaccggctttagccggtcgtccgccattaacgaggaggatgggtcttcagaagagggggttgtgattaatgccggaaaggatagcaccagcagagagcttcccagtgcgactcccaatacagcggggagttccagcacgaggtttatagccaatagtcaggagcctgaaatcaggctgacttcaagcctgccgcgctcccccgggaggtctactgaggacctgccaggctcgcaggccaccctgagccagtggtccacacctgggtctaccccgagccggtggccgtcaccctcacccacagccatgccatctcctgaggatctgcggctggtgctgatgccctggggcccgtggcactgccactgcaagtcgggcaccatgagccggagccggtctgggaagctgcacggcctttccgggcgccttcgagttggggcgctgagccagctccgcacggagcacaagccttgcacctatcaacaatgtccctgcaaccgacttcgggaagagtgccccctggacacaagtctctgtactgacaccaactgtgcctctcagagcaccaccagtaccaggaccaccactacccccttccccaccatccacctcagaagcagtcccagcctgccacccgccagcccctgcccagccctggctttttggaaacgggtcaggattggcctggaggatatttggaatagcctctcttcagtgttcacagagatgcaaccaatagacagaaaccagaggtaa

SEQ ID NO: 211 - DNA, 1077 bp, Homo sapiens
atgaagacctgctggaaaattccagttttcttctttgtgtgcagtttcctggaaccctgggcatctgcagaattcgaattccggcgaaggtttccgtttgtccgggagaggagtgactccactggctccagctctgtctacttcacggcctcctcgggagccacgttcacagatgctgagagtgaagggggttacacaacagccaatgcggagtctgacaatgagcgggactctgacaaagaaagtgaggacggggaagatgaagtgagctgtgagactgtgaagatggggagaaaggattctcttgacttggaggaagaggcagcttcaggtgcctccagtgccctggaggctggaggttcctcaggcttggaggatgtgctgcccctcctgcagcaggccgacgagctgcacaggggtgatgagcaaggcaagcgggagggcttccagctgctgctcaacaacaagctggtgtatggaagccggcaggactttctctggcgcctggcccgagcctacagtgacatgtgtgagctcactgaggaggtgagcgagaagaagtcatatgccctagatggaaaagaagaagcagaggctgctctggagaagggggatgagagtgctgactgtcacctgtggtatgcggtgctttgtggtcagctggctgagcatgagagcatccagaggcgcatccagagtggctttagcttcaaggagcatgtggacaaagccattgctctccagccagaaaaccccatggctcactttcttcttggcaggtggtgctatcaggtctctcacctgagctggctagaaaaaaaaactgctacagccttgcttgaaagccctctcagtgccactgtggaagatgccctccagagcttcctaaaggctgaagaactacagccaggattttccaaagcaggaagggtatatatttccaagtgctacagagaactagggaaaaactctgaagctagatggtggatgaagttggccctggagctgccagatgtcacgaaggaggatttggctatccagaaggacctggaagaactggaagtcattttacgagac

SEQ ID NO: 212 - DNA, 387 bp, Homo sapiens
atgtcacttatcctcctttgtccacaaagacttgagggcctcctgagtctccaagcatggaggaatatccatgccaacatccctgccatcaaactcaatccattttcttcagagataccttgtctgtcaccagcctccaacttcatattccttccacaggcaactgtgcacttgacccaaggtaaaatgaaagggctgccaatcactcggactccagaaggtgcctgccctgagaagagctggcatgttacctcccacatacatttcctgtcatcctgtccgaatctgggaaattttgctctggagtattttcaagaaagcgcattgtgttttaatgaagtatttttcagggttccaatgttattttttataaatgcagcattttaa

SEQ ID NO: 213 - DNA, 453 bp, Homo sapiens
  modified_base (86)..(86): a, c, g, t, unknown or other
atgctgatgatctctgatggaggctgctgggtggctgctgccctcaccctgacctgtcctccccagctggacgggggacttctagngnangggggagggtccccactgaagatggacacatgtcctgtcagagtttctgagaagctccagcccttaggcagaatgggacaaggagaaacagacttgggtctgtactcgggactgcagaagccaagcatgaggagttgtggggacagtgcccctccttcatccccacctcccagccccccagtatctccttctccccctgatccttcccttgatcaggccagggccctgtcattgtcccaacaaatgcctaccgccagttcattgcaccttgttttctctgatgtcctcgcaatcacacaccttgtgcccctgagctgggtacacgatcctttgcaatgtgtgctacgagaactgtattgctaa

SEQ ID NO: 214 - DNA, 747 bp, Homo sapiens
atgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgac

SEQ ID NO: 215 - DNA, 549 bp, Homo sapiens
atgaagacctgctggaaaattccagttttcttctttgtgtgcagtttcctggaaccctgggcatctgcagaattcgcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 216 - DNA, 686 bp, Homo sapiens
atggtctgcggcagcccgggagggatgctgctgctgcgggccgggctgcttgccctggctgctctctgcctgctccgggtgcccggggctcgggctgcagcctgtgagcccgtccgcatccccctgtgcaagtccctgccctggaacatgactaagatgcccaaccacctgcaccacagcactcaggccaacgccatcctggccatcgagcagttcgaaggtctgctgggcacccactgcagccccgatctgctcttcttcctctgtgccatgtacgcgcccatctgcaccattgacttccagcacgagcccatcaagccctgtaagtctgtgtgcgagcgggcccggcagggctgtgagcccatactcatcaagtaccgccactcgtggccggagaacctggcctgcgaggagctgccagtgtacgacaggggcgtgtgcatctctcccgaggccatcgttactgcggacggagctgattttcctatggattctagtaacggaaactgtagaggggcaagcagtgtcattcgggctaaagttaaagagataaagactaagtgccatgatgtgactgcagtagtggaggtgaaggagattctaaagtcctctctggtaaacattccacgggacactgtcaacctctataccagctctggctgcctctgccctccactttatgttaa

SEQ ID NO: 217 - DNA, 762 bp, Homo sapiens
atggtgatgtcccagggcacctacacgttcctcacgtgcttcgccggcttctggctcatctggggtctcatcgtcctgctctgctgcttctgcagcttcctgcgccgccgcctcaaacggcgccaggaggagcgactgcgcgagcagaacctgcgcgccctagagctggagcccctcgaactcgagggcagtctggccgggagccccccgggcctggcgccgccgcagccaccaccacaccgtagccgcctggaggcgccggctcacgcgcacgcgcacccacacccgcaccaccacgcgctcccgcacccgccgcctacgcacctgtcggtgccgccacggccctggagctacccgcgccaagcggaatcggacatgtccaaaccaccgtgttacgaagaggcggtgctgatggcagagccgccgccgccctatagcgaggtgctcacggacacgcgcggcctctaccgcaagatcgtcacgcccttcctgagtcgccgcgacagcgcggagaagcaggagcagccgcctcccagctacaagccgctcttcctggaccggggctacacctcggcgctgcacctgcccagcgcccctcggcccgcgccgccctgcccagccctctgcctgcaggccgaccgtggccgccgggtcttccccagctggaccgactcagagctcagcagccgcgagcccctggagcacggagcttggcgtctgccggtctccatccccttgttcgggaggactacagccgtatag

SEQ ID NO: 218 - DNA, 435 bp, Homo sapiens
atgggtgctgtggtcctctgcaggccctcaccccttaacttcctcatacagactggcactgggcagggcctctcatgtggcagccacatgtggcgttgtgaggccaccccatgtggggtctgtggtgagagtcctgtaggatccctgctcaagcagcacagaggaaggggcaagacgtggcctgtaggcactgtctcagcctgcagagaagaaagtgaggccgggagcctgagcctgggctggagccttctcccctccccagttggactaggggcagtgttaattttgaaaaggtgtgggtccctgtgtcctcttccaggggtccaagggaacaggagaggtcactgggcctgttttctccctcctgaccctgcatctcccaccccgtgtatcatagggaactttcaccttaaaatctttctaagcaaagtgtga

SEQ ID NO: 219 - DNA, 690 bp, Homo sapiens
atgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccagctacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtag

SEQ ID NO: 220 - DNA, 366 bp, Homo sapiens
atgatgctaccccaatggctgctgctgctgttccttctcttcttctttctcttcctcctcaccaggggctcactttctccaacaaaatacaaccttttggagctcaaggagtcttgcatccggaaccaggactgcgagactggctgctgccaacgtgctccagacaattgcgagtcgcactgcgcggagaaggggtccgagggcagtctgtgtcaaacgcaggtgttctttggccaatatagagcgtgtccctgcctgcggaacctgacttgtatatattcaaagaatgagaaatggcttagcatcgcctatggccgttgtcagaaaattggaaggcagaagttggctaagaaaatgttcttctag

SEQ ID NO: 221 - DNA, 276 bp, Homo sapiens
atgggttggagtattctgactgcggttgctataatttggctgctcatcagttttggtctggctcagccgcgctatctttcgtccctagttttaagcctgcctgaaaatcgacaaacagatgaatggacttcacagcttttatctattcatggtattgaagaagtcgtggtgatgcctgaacagcaagtggcctacgttaaagttgataaacagcagattaatgatactgcgcgacaacaattaacgcacttgttgggtaaagaggtagccatttaa

SEQ ID NO: 222 - DNA, 690 bp, Homo sapiens
atgaacttggaggggcttgaaatgatagcagttctgatcgtcattgtgctttttgttaaattattggaacagtttgggctgattgaagcaggtttagaagacagcgtggaagatgagctggagatggctactgtcaggcatcggcctgaagccctggagctgctggaggcccagagcaaattcaccaagaaagagcttcagattctttacagaggatttaagaatgaatgccccagtggtgttgttaatgaagaaactttcaaggagatttactcacagttctttccacagggagactccaccacatatgcacattttctcttcaatgcattcgacacggaccacaatggagctgtgagctttgaggatttcatcaaaggtctttccattttgcttcgagggacagtacaagaaaaactcaactgggcatttaatttgtatgacataaacaaagatggctacatcactaaagaagaaatgctggacataatgaaagcaatctacgacatgatggggaaatgcacatacccggtcctcaaggaagatgctccccgacagcatgtggagacgttcttccagaagatggacaaaaataaagatggtgtcgttaccatagatgagttcattgaaagttgccaaaaagatgaaaacataatgcgctccatgcagctctttgaaaatgtgatctag

SEQ ID NO: 223 - DNA, 627 bp, Homo sapiens
atggacccgttcctggtgctgctgcactcggtgtcgtccagcctgtcgagcagcgagctgaccgagctcaagttcctatgcctcgggcgcgtgggcaagcgcaagctggagcgcgtgcagagcggcctagacctcttctccatgctgctggagcagaacgacctggagcccgggcacaccgagctcctgcgcgagctgctcgcctccctgcggcgccacgacctgctgcggcgcgtcgacgacttcgaggcgggggcggcggccggggccgcgcctggggaagaagacctgtgtgcagcatttaacgtcatatgtgataatgtggggaaagattggagaaggctggctcgtcagctcaaagtctcagacaccaagatcgacagcatcgaggacagatacccccgcaacctgacagagcgtgtgcgggagtcactgagaatctggaagaacacagagaaggagaacgcaacagtggcccacctggtgggggctctcaggtcctgccagatgaacctggtggctgacctggtacaagaggttcagcaggcccgtgacctccagaacaggagtggggccatgtccccgatgtcatggaactcagacgcatctacctccgaagcgtcctga

SEQ ID NO: 224 - DNA, 330 bp, Homo sapiens
atgctgtggtggctagtgctcctactcctacctacattaaaatctgttttttgttctcttgtaactagcctttaccttcctaacacagaggatctgtcactgtggctctggcccaaacctgaccttcactctggaacgagaacagaggtttctacccacaccgtcccctcgaagccggggacagcctcaccttgctggcctctcgctggagcagtgccctcaccaactgtctcacgtctggaggcactgactcgggcagtgcaggtagctgagcctcttggtagctgcggctttcaaggtgggccttgccctggccgtagaagggattga

SEQ ID NO: 225 - DNA, 2853 bp, Homo sapiens
atgacatggagaatgggaccccgtttcactatgctgttggccatgtggctagtgtgtggatcagaaccccacccccatgccactattagaggcagccacggaggacggaaagtgcctttggtttctccggacagcagtaggccagctcggtttctgaggcacactgggaggtctcgcggaattgagagatccactctggaggaaccaaaccttcagcctctccagagaaggaggagtgtgcccgtgttgagactagctcgcccaacagagccgccagcccgctcggacatcaatggggccgccgtgagacctgagcaaagaccagcagccaggggctctccgcgtgagatgatcagagatgaggggtcctcagctcggtcaagaatgttgcgtttcccttcggggtccagctctcccaacatccttgccagctttgcagggaagaacagagtatgggtcatctcagcccctcatgcctcggaaggctactaccgcctcatgatgagcctgctgaaggacgatgtgtactgtgagctggcggagaggcacatccaacagattgtgctcttccaccaggcaggtgaggaaggaggcaaggtgagaaggatcaccagcgagggccagatcctggagcagcccctggaccctagcctcatccctaagctgatgagcttcctgaagctggagaagggcaagtttggcatggtgctgctgaagaagacgctgcaggtggaggagcgctatccatatcccgttaggctggaagccatgtacgaggtcatcgaccaaggccccatccgtaggatcgagaagatcaggcagaagggctttgtccagaaatgtaaggcctctggtgtagagggccaggtggtggcggaggggaatgacggtggagggggagcaggaaggccaagcctgggcagcgagaagaagaaagaggacccaaggagagcacaagtcccaccaaccagagagagtcgggtgaaggtcctgagaaaactggccgccactgcaccagctttgccccaacctccctcaacccccagagccaccacccttcctcctgccccagccacaacagtgactcggtccacgtcccgggcggtaacagttgctgcaagacctatgaccaccactgcctttcccaccacgcagaggccctggaccccctcaccctcccacaggccccctacaaccactgaggtgatcactgccaggagaccctcagtttcagagaatctttaccctccatcccggaaggatcagcacagggagaggccacagacaaccaggaggcccagcaaggccaccagcttggagagcttcacaaatgcccctcccaccaccatctcagaacccagcacaagggctgctggcccaggccgtttccgggacaaccgcatggacaggcgggaacatggccaccgagacccaaatgtggtgccaggtcctcccaagccagcaaaggagaaacctcccaaaaagaaggcccaggacaaaattcttagtaatgagtatgaggagaagtatgacctcagccggcctactgcctctcagctggaggacgagctgcaggtggggaatgttccccttaaaaaagcaaaggagtctaaaaagcatgaaaagcttgagaaaccagagaaggagaagaaaaaaaagatgaagaatgagaacgcagacaagttacttaagagtgaaaagcaaatgaagaagtctgagaaaaagagcaagcaagagaaagagaagagcaagaagaaaaaaggaggtaaaacagaacaggatggctatcagaaacccaccaacaaacacttcacgcagagtcccaagaagtcagtggccgacctgctggggtcctttgaaggcaaacgaagactccttctgatcactgctcccaaggctgagaacaatatgtatgtgcaacaacgtgatgaatatctggaaagtttctgcaagatggctaccaggaaaatctctgtgatcaccatcttcggccctgtcaacaacagcaccatgaaaatcgaccactttcagctagataatgagaagcccatgcgagtggtggatgatgaagacttggtagaccagcgtctcatcagcgagctgaggaaagagtacggaatgacctacaatgacttcttcatggtgctaacagatgtggatctgagagtcaagcaatactatgaggtaccaataacaatgaagtctgtgtttgatctgatcgatactttccagtcccgaatcaaagatatggagaagcagaagaaggagggcattgtttgcaaagaggacaaaaagcagtccctggagaacttcctatccaggttccggtggaggaggaggttgctggtgatctctgctcctaacgatgaagactgggcctattcacagcagctctctgccctcagtggtcaggcgtgcaattttggtctgcgccacataaccattctgaagcttttaggcgttggagaggaagttgggggagtgttagaactgttcccaattaatgggagctctgttgttgagcgagaagacgtaccagcccatttggtgaaagacattcgtaactattttcaagtgagcccggagtacttctccatgcttctagtcggaaaagacggaaatgtcaaatcctggtatccttccccaatgtggtccatggtgattgtgtacgatttaattgattcgatgcaacttcggagacaggaaatggcgattcagcagtcactggggatgcgctgcccagaagatgagtatgcaggctatggttaccatagttaccaccaaggataccaggatggttaccaggatgactaccgtcatcatgagagttatcaccatggatacccttactga

SEQ ID NO: 226 - DNA, 207 bp, Homo sapiens
atgttagaactactcaggactctgggcttttactatatactccatttcctaggcagtctcatacatgccctgggctttaaataccatctatctgatagtatctctaatatagatctgtcttctgagttccagaacttagcaattctattagatgtctcctgggtattgcaatactcagtaagttcatatctgacagagaccttctaa